(19) United States
(12) Patent Application Publication
(10) Pub. No.: US 2014/0200188 A1
(43) Pub. Date: Jul. 17, 2014

(54) 2'-FLUORO SUBSTITUTED CARBA-NUCLEOSIDE ANALOGS FOR ANTIVIRAL TREATMENT

(71) Applicant: Gilead Sciences, Inc., Foster City, CA (US)

(72) Inventors: Michael O'Neil Hanrahan Clarke, Redwood City, CA (US); Choung U. Kim, San Carlos, CA (US); Willard Lew, San Mateo, CA (US)

(21) Appl. No.: 14/163,251

(22) Filed: Jan. 24, 2014

Related U.S. Application Data
(63) Continuation of application No. 13/230,634, filed on Sep. 12, 2011.
(60) Provisional application No. 61/382,145, filed on Sep. 13, 2010.

(51) Int. Cl. C07H 17/02; A61K 45/06; A61K 31/706

(52) U.S. Cl. CPC C07H 17/02; A61K 31/706; A61K 45/06
USPC 514/23; 424/85.4; 536/29.2

(57) ABSTRACT

Provided are methods for treating Orthomyxoviridue virus infections by administering ribosides, riboside phosphates and prodrugs thereof, of Formula I:

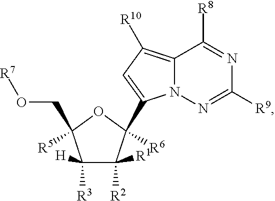

wherein R² is halogen. The compounds, compositions, and methods provided are particularly useful for the treatment of Human Influenza virus infections.

### CROSS-REFERENCE TO RELATED APPLICATIONS

The present application is a continuation of U.S. patent application Ser. No. 13/230,634, filed Sep. 12, 2011, which claims the benefit under 35 U.S.C. §119(e) of U.S. Provisional Application No. 61/382,145, filed Sep. 13, 2010. The foregoing applications are incorporated herein by reference in their entireties.

### FIELD OF THE INVENTION

The invention relates generally to compounds with antiviral activity, more particularly nucleosides active against Orthomyxoviridue virus infections.

### BACKGROUND OF THE INVENTION

Influenza viruses of the Orthomyxoviridae family that belong to the genera A and B are responsible for seasonal flu epidemics each year, which cause acute contagious respiratory infections. Children, the old, and people with chronic diseases are at high risk to develop severe complications that lead to high morbidity and mortality rates (Memoli et al., Drug Discovery Today 2008, 13, 590-595). Among the three influenza genera, type A viruses are the most virulent human pathogens that cause the most severe disease, can be transmitted to other species, and give rise to human influenza pandemics. The recent human influenza outbreak of the aggressive porcine A/H1N1 strain in 2009 has emphasized the need for novel antiviral therapeutics. While yearly vaccination programs are currently used to protect populations form influenza infection, these programs must anticipate the virus strains that will be prevalent during seasonal outbreaks to be effective and they do not address the problem of sudden, unanticipated influenza pandemics. The recent human influenza outbreak of the aggressive porcine A/H1N1 strain in 2009 is an example of this problem.

Several anti-influenza therapeutics are now available and others are under development (Hedlund, et al., Viruses 2010, 2, 1766-1781). Among the currently available anti-influenza therapeutics are the M2 ion channel blockers amantadine and rimantadine and the neuraminidase inhibitors oseltamivir and zanamivir. However, resistance has developed to all of these medications. Therefore there is a continuing need for novel anti-influenza therapeutics.

Promising new anti-influenza agents with novel mechanisms of action are now in development. Among these new agents is favipiravir that targets viral gene replication by inhibiting influenza RNA polymerase. However, it is still uncertain whether this investigational drug candidate will become available for therapy. Therefore, there is a continuing need to develop additional compounds that inhibit influenza through this mechanism of action.

Certain ribosides of the nucleobases pyrrolo[1,2-f][1,2,4]triazine, imidazo[1,5-f][1,2,4]triazine, imidazo[1,2-f][1,2,4]triazine, and [1,2,4]triazolo[4,3-f][1,2,4]triazine have been disclosed in Carbohydrate Research 2001, 331(1), 77-82; Nucleosides & Nucleotides (1996), 15(1-3), 793-807; Tetrahedron Letters (1994), 35(30), 5339-42; Heterocycles (1992), 34(3), 569-74; J. Chem. Soc. Perkin Trans. 1 1985, 3, 621-30; J. Chem. Soc. Perkin Trans. 1 1984, 2, 229-38; WO 2000056734; Organic Letters (2001), 3(6), 839-842; J. Chem. Soc. Perkin Trans. 1 1999, 20, 2929-2936; and J. Med. Chem. 1986, 29(11), 2231-5. However, these compounds have not been disclosed as useful for the treatment of Orthomyxoviridae infections.

Ribosides of pyrrolo[1,2-f][1,2,4]triazinyl, imidazo[1,5-f][1,2,4]triazinyl, imidazo[1,2-f][1,2,4]triazinyl, and [1,2,4]triazolo[4,3-f][1,2,4]triazinyl nucleobases with antiviral, anti-HCV, and anti-RdRp activity have been disclosed by Babu, Y. S., WO2008/089105 and WO2008/141079; Cho, et al., WO2009/132123 and Francom, et al. WO2010/002877. Butler, et al., WO2009/132135, has disclosed anti-viral pyrrolo[1,2-f][1,2,4]triazinyl, imidazo[1,5-f][1,2,4]triazinyl, imidazo[1,2-f][1,2,4]triazinyl, and [1,2,4]triazolo[4,3-f][1,2,4]triazinyl nucleosides wherein the 1′ position of the nucleoside sugar is substituted with a cyano or methyl group. However, the effectiveness of these compounds for the treatment of Orthomyxoviridae infections has not been disclosed.

### SUMMARY OF THE INVENTION

Provided are compounds that inhibit viruses of the Orthomyxoviridae family. The invention also comprises compounds of Formula I that inhibit viral nucleic acid polymerases, particularly Orthomyxoviridae RNA-dependent RNA polymerase (RdRp), rather than cellular nucleic acid polymerases. Compounds of Formula I are useful for treating Orthomyxoviridae infections in humans and other animals.

Provided, is a method for treating a Orthomyxoviridae infection in a mammal in need thereof comprising administering a therapeutically effective amount of a compound of Formula I:

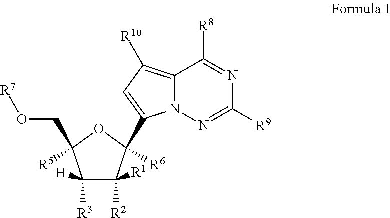

or a pharmaceutically acceptable salt or ester, thereof;

wherein:

each R¹ is H or halogen;

each R² is halogen;

each R³ or R⁵ is independently H, OR^{a}, N(R^{a})₂, N₃, CN, NO₂, S(O)ₙR^{a}, halogen, (C₁-C₈)alkyl, (C₄-C₈)carbocyclylalkyl, (C₁-C₈)substituted alkyl, (C₂-C₈)alkenyl, (C₂-C₈)substituted alkenyl, (C₂-C₈)alkynyl or (C₂-C₈)substituted alkynyl;

R⁶ is H, OR^{a}, N(R^{d})₂, N₃, CN, NO₂, S(O)ₙR^{a}, —C(═O)R¹¹, —C(═O)OR¹¹, —C(═O)NR¹¹R¹², —C(═O)SR¹¹, —S(O)R¹¹, —S(O)₂R¹¹, —S(O)(OR¹¹), —S(O)₂(OR¹¹), —SO₂NR¹¹R¹², halogen, (C₁-C₈)alkyl, (C₄-C₈)carbocyclylalkyl, (C₁-C₈)substituted alkyl, (C₂-C₈)alkenyl, (C₂-C₈)substituted alkenyl, (C₂-C₈)alkynyl, (C₂-C₈)substituted alkynyl, or aryl(C₁-C₈)alkyl;

each n is independently 0, 1, or 2;

each R^{a} is independently H, (C₁-C₈)alkyl, (C₂-C₈)alkenyl, (C₂-C₈)alkynyl, aryl(C₁-C₈)alkyl, (C₄-C₈)carbocyclylalkyl, —C(═O)R¹¹, —C(═O)OR¹¹, —C(═O)NR¹¹R¹², —C(═O)SR¹¹, —S(O)R¹¹, —S(O)₂R¹¹, —S(O)(OR¹¹), —S(O)₂(OR¹¹), or —SO₂NR¹¹R¹²;

R⁷ is H, —C(═O)R¹¹, —C(═O)OR¹¹, —C(═O)NR¹¹R¹², —C(═O)SR¹¹, —S(O)R¹¹, —S(O)₂R¹¹, —S(O)(OR¹¹), —S(O)₂(OR¹¹), —SO₂NR¹¹R¹², or

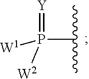

each Y or Y¹ is, independently, O, S, NR, ⁺N(O)(R), N(OR), ⁺N(O)(OR), or N—NR₂;

W¹ and W², when taken together, are —Y³(C(R^{y})₂)₃Y³—; or one of W¹ or W² together with either R³ is —Y³— and the other of W¹ or W² is Formula Ia; or W¹ and W² are each, independently, a group of the Formula Ia:

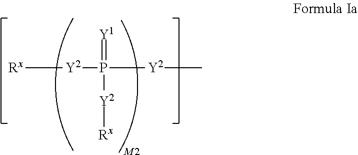

wherein:

each Y² is independently a bond, O, CR₂, NR, ⁺N(O)(R), N(OR), ⁺N(O)(OR), N—NR₂, S, S—S, S(O), or S(O)₂;

each Y³ is independently O, S, or NR;

M2 is 0, 1 or 2;

each R^{x} is independently R^{y} or the formula:

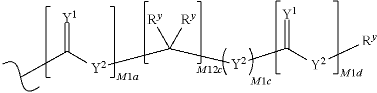

wherein:

each M1a, M1c, and M1d is independently 0 or 1;

M12c is 0, 1, 2, 3, 4, 5, 6, 7, 8, 9, 10, 11 or 12;

each R^{y} is independently H, F, Cl, Br, I, OH, R, —C(═Y¹)R, —C(═Y¹)OR, —C(═Y¹)N(R)₂, —N(R)₂, —⁺N(R)₃, —SR, —S(O)R, —S(O)₂R, —S(O)(OR), —S(O)₂(OR), —OC(═Y¹)R, —OC(═Y¹)OR, —OC(═Y¹)(N(R)₂), —SC(═Y¹)R, —SC(═Y¹)OR, —SC(═Y¹)(N(R)₂), —N(R)C(═Y¹)R, —N(R)C(═Y¹)OR, —N(R)C(═Y¹)N(R)₂, —SO₂NR₂, —CN, —N₃, —NO₂, —OR, or W³; or when taken together, two R^{y} on the same carbon atom form a carbocyclic ring of 3 to 7 carbon atoms;

each R is independently H, (C₁-C₈) alkyl, (C₁-C₈) substituted alkyl, (C₂-C₈)alkenyl, (C₂-C₈) substituted alkenyl, (C₂-C₈) alkynyl, (C₂-C₈) substituted alkynyl, C₆-C₂₀ aryl, C₆-C₂₀ substituted aryl, C₂-C₂₀ heterocyclyl, C₂-C₂₀ substituted heterocyclyl, arylalkyl or substituted arylalkyl;

W³ is W⁴ or W⁵; W⁴ is R, —C(Y¹)R, —C(Y¹)W⁵, —SO₂R^{y}, or —SO₂W⁵; and W⁵ is a carbocycle or a heterocycle wherein W⁵ is independently substituted with 0 to 3 R^{y} groups;

each R⁸ is halogen, NR¹¹R¹², N(R¹¹)OR¹¹, NR¹¹NR¹¹R¹², N₃, NO, NO₂, CHO, CN, —CH(═NR¹¹), —CH═NNHR¹¹, —CH═N(OR¹¹), —CH(OR¹¹)₂, —C(═O)NR¹¹R², —C(═S)NR¹¹R¹², —C(═O)OR¹¹, (C₁-C₈)alkyl, (C₂-C₈)alkenyl, (C₂-C₈)alkynyl, (C₄-C₈)carbocyclylalkyl, optionally substituted aryl, optionally substituted heteroaryl, —C(═O)(C₁-C₈)alkyl, —S(O)ₙ(C₁-C₈)alkyl, aryl(C₁-C₈)alkyl, OR¹¹ or SR¹¹;

each R⁹ or R¹⁰ is independently H, halogen, NR¹¹R¹², N(R¹¹)OR¹¹, NR¹¹NR¹¹R¹², N₃, NO, NO₂, CHO, CN, —CH(═NR¹¹), —CH═NHNR¹¹, —CH═N(OR¹¹), —CH(OR¹¹)₂, —C(═O)NR¹¹R¹², —C(═S)NR¹¹R¹², —C(═O)OR¹¹, R¹¹, OR¹¹ or SR¹¹;

each R¹¹ or R¹² is independently H, (C₁-C₈)alkyl, (C₂-C₈)alkenyl, (C₂-C₈)alkynyl, (C₄-C₈)carbocyclylalkyl, optionally substituted aryl, optionally substituted heteroaryl, —C(═O)(C₁-C₈)alkyl, —S(O)ₙ(C₁-C₈)alkyl or aryl(C₁-C₈)alkyl; or R¹¹ and R¹² taken together with a nitrogen to which they are both attached form a 3 to 7 membered heterocyclic ring wherein any one carbon atom of said heterocyclic ring can optionally be replaced with —O—, —S— or —NR^{a}—; and

wherein each (C₁-C₈)alkyl, (C₂-C₈)alkenyl, (C₂-C₈)alkynyl or aryl(C₁-C₈)alkyl of each R³, R⁵, R⁶, R¹¹ or R¹² is, independently, optionally substituted with one or more halo, hydroxy, CN, N₃, N(R^{a})₂ or OR^{a}; and wherein one or more of the non-terminal carbon atoms of each said (C₁-C₈)alkyl may be optionally replaced with —O—, —S— or —NR^{a}—.

In another embodiment, the method comprises administering a therapeutically effective amount of a racemate, enantiomer, diastereomer, tautomer, polymorph, pseudopolymorph, amorphous form, hydrate or solvate of a compound of Formula I or a pharmaceutically acceptable salt or ester thereof to a manual in need thereof.

In another embodiment, the method comprises treating a Orthomyxoviridae infection in a mammal in need thereof by administering a therapeutically effective amount of a compound of Formula I or a pharmaceutically acceptable salt or ester thereof. In another aspect of this embodiment, the Orthomyxoviridae infection is a Influenzavirus A infection. In another aspect of this embodiment, the Orthomyxoviridae infection is a Influenzavirus B infection. In another aspect of this embodiment, the Orthomyxoviridae infection is a Influenzavirus C infection.

In another embodiment, the method comprises treating a Orthomyxoviridae infection in a mammal in need thereof by administering a therapeutically effective amount of a pharmaceutical composition comprising an effective amount of a Formula I compound, or a pharmaceutically acceptable salt or ester thereof, in combination with a pharmaceutically acceptable diluent or carrier. In another aspect of this embodiment, the Orthomyxoviridae infection is a Influenza virus A infection. In another aspect of this embodiment, the Orthomyxoviridae infection is a Influenza virus B infection. In another aspect of this embodiment, the Orthomyxoviridae infection is a Influenza virus C infection.

In another embodiment, the method comprises treating a Orthomyxoviridae infection in a mammal in need thereof by administering a therapeutically effective amount of a pharmaceutical composition comprising an effective amount of a Formula I compound, or a pharmaceutically acceptable salt or ester thereof, in combination with at least one additional therapeutic agent. In another aspect of this embodiment, the Orthomyxoviridae infection is a Influenza virus A infection. In another aspect of this embodiment, the Orthomyxoviridae infection is a Influenza virus B infection. In another aspect of this embodiment, the Orthomyxoviridae infection is a Influenza virus C infection.

In another embodiment, the present application provides for a method of inhibiting a Orthomyxoviridae RNA-dependent RNA polymerase, comprising contacting a cell infected with Orthomyxoviridae virus with an effective amount of a compound of Formula I; or a pharmaceutically acceptable salts, solvate, and/or ester thereof. In another aspect of this embodiment, the Orthomyxoviridae RNA-dependent RNA polymerase is a Influenza virus A RNA-dependent RNA polymerase. In another aspect of this embodiment, the Orthomyxoviridae RNA-dependent RNA polymerase is a Influenza virus B RNA-dependent RNA polymerase. In another aspect of this embodiment, the Orthomyxoviridae RNA-dependent RNA polymerase is a Influenza virus C RNA-dependent RNA polymerase.

In another embodiment, provided is the use of a compound of Formula I or a pharmaceutically acceptable salt, solvate, and/or ester thereof to treat a viral infection caused by a Orthomyxoviridae virus.

In another embodiment, the present application provides for combination pharmaceutical agent comprising:

a) a first pharmaceutical composition comprising a compound of Formula I; or a pharmaceutically acceptable salt, solvate, or ester thereof; and

b) a second pharmaceutical composition comprising at least one additional therapeutic agent active against infectious Orthomyxoviridae viruses.

In another aspect of this embodiment, the additional therapeutic agent is a viral haemagglutinin inhibitor, a viral neuramidase inhibitor, a M2 ion channel inhibitor, a Orthomyxoviridae RNA-dependent RNA polymerase inhibitor or a sialidase. In another aspect of this embodiment, the additional therapeutic agent is selected from the group consisting of ribavirin, oseltamivir, zanamivir, laninamnivir, peramivir, amantadine, rimantadine, CS-8958, favipiravir, AVI-7100, alpha-1 protease inhibitor and DAS181.

In another embodiment, the present application provides for a method of treating a Orthomyxoviridae virus infection in a patient, comprising administering to said patient a therapeutically effective amount of a compound of Formula I; or a pharmaceutically acceptable salt, solvate, and/or ester thereof. In another aspect of this embodiment, the Orthomyxoviridae virus is Influenza virus A. In another aspect of this embodiment, the Orthomyxoviridae virus is Influenza virus B. In another aspect of this embodiment, the Orthomyxoviridae virus is Influenza virus C.

In another embodiment, the present application provides for a method of treating a Orthomyxoviridae virus infection in a patient, comprising administering to said patient a therapeutically effective amount of a compound of Formula I; or a pharmaceutically acceptable salt, solvate, and/or ester thereof; and at least one additional therapeutic agent. In another aspect of this embodiment, the additional therapeutic agent is selected from the group consisting of ribavirin, oseltamivir, zanamivir, laninamivir, peramivir, amantadine, rimantadine, CS-8958, favipiravir, AVI-7100, alpha-1 protease inhibitor and DAS181.

In another aspect, the invention also provides processes and novel intermediates disclosed herein which are useful for preparing Formula I compounds of the invention.

In other aspects, novel methods for synthesis, analysis, separation, isolation, purification, characterization, and testing of the compounds of this invention are provided.

### DETAILED DESCRIPTION OF EXEMPLARY EMBODIMENTS

Reference will now be made in detail to certain embodiments of the invention, examples of which are illustrated in the accompanying description, structures and formulas. While the invention will be described in conjunction with the enumerated embodiments, it will be understood that they are not intended to limit the invention to those embodiments. On the contrary, the invention is intended to cover all alternatives, modifications, and equivalents, which may be included within the scope of the present invention.

In another embodiment, provided is a method of treating a Orthomyxoviridae infection in a mammal in need thereof comprising administering a therapeutically effective amount of a compound of Formula I represented by Formula II:

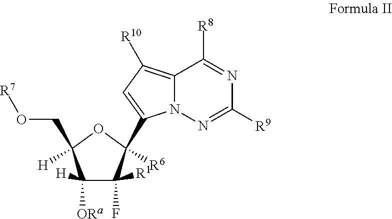

or a pharmaceutically acceptable salt or ester, thereof;

wherein the variables are defined as for Formula I.

In one embodiment of the invention the method of treating a Orthomyxoviridae infection by administering a compound of Formula II, R¹ is H. In another aspect of this embodiment, R⁶ is H, CN, halogen, (C₁-C₈)alkyl, (C₁-C₈)substituted alkyl, (C₂-C₈)alkenyl, (C₂-C₈)substituted alkenyl, (C₂-C₈)alkynyl or (C₂-C₈)substituted alkynyl. In another aspect of this embodiment, R⁶ is H, CN, methyl, ethenyl, or ethynyl. In another aspect of this embodiment, R⁶ is H. In another aspect of this embodiment, R⁶ is CN. In another aspect of this embodiment, R⁶ is methyl. In another aspect of this embodiment, R⁶ is ethenyl. In another aspect of this embodiment, R⁶ is ethynyl. In another aspect of this embodiment, R¹⁰ is H, halogen, CN, CHO, or optionally substituted heteroaryl. In another aspect of this embodiment, R¹⁰ is H, halogen or CN. In another aspect of this embodiment, R¹⁰ is H. In another aspect of this embodiment, R¹⁰ is halogen. In another aspect of this embodiment, R⁸ is NR¹¹R¹². In another aspect of this embodiment, R⁸ is NH₂. In another aspect of this embodiment, R⁸ is OR¹¹. In another aspect of this embodiment, R⁸ is OH. In another aspect of this embodiment, R⁹ is H. In another aspect of this embodiment, R⁹ is NR¹¹R¹². In another aspect of this embodiment, R⁹ is NH₂. In another aspect of this embodiment, R^{a} is H, —C(═O)R¹¹ or —C(═O)OR¹¹. In another aspect of this embodiment, R^{a} is H. In another aspect of this embodiment, R⁷ is H, —C(═O)R¹¹, —C(═O)OR¹¹ or

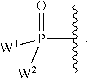

In another aspect of this embodiment, R⁷ is H. In another aspect of this embodiment, R⁷ is

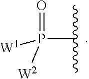

In one embodiment of the invention the method of treating a Orthomyxoviridae infection by administering a compound of Formula II, R¹ is F. In another aspect of this embodiment, R⁶ is H. CN, halogen, (C₁-C₈)alkyl, (C₁-C₈)substituted alkyl, (C₂-C₈)alkenyl, (C₂-C₈)substituted alkenyl, (C₂-C₈)alkynyl or (C₂-C₈)substituted alkynyl. In another aspect of this embodiment, R⁶ is H, CN, methyl, ethenyl, or ethynyl. In another aspect of this embodiment, R⁶ is H. In another aspect of this embodiment, R⁶ is CN. In another aspect of this embodiment, R⁶ is methyl. In another aspect of this embodiment, R⁶ is ethenyl. In another aspect of this embodiment, R⁶ is ethynyl. In another aspect of this embodiment. R¹⁰ is H, halogen, CN, CHO, or optionally substituted heteroaryl. In another aspect of this embodiment, R¹⁰ is H, halogen or CN. In another aspect of this embodiment, R¹⁰ is H. In another aspect of this embodiment. R¹⁰ is halogen. In another aspect of this embodiment, R⁸ is NR¹¹R¹². In another aspect of this embodiment, R⁸ is NH₂. In another aspect of this embodiment. R⁸ is OR¹¹. In another aspect of this embodiment, R⁸ is OH. In another aspect of this embodiment, R⁹ is H. In another aspect of this embodiment, R⁹ is NR¹¹R¹². In another aspect of this embodiment, R⁹ is NH₂. In another aspect of this embodiment, R^{a} is H, —C(═O)R¹¹ or —C(═O)OR¹¹. In another aspect of this embodiment, R^{a} is H. In another aspect of this embodiment, R⁷ is H, —C(═O)R¹¹, —C(═O)OR¹¹ or

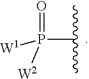

In another aspect of this embodiment, R⁷ is H. In another aspect of this embodiment, R⁷ is

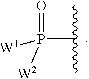

In one embodiment of the invention the method of treating a Orthomyxoviridae infection by administering a compound of Formula II, each R¹ and R⁶ is H. In another aspect of this embodiment, R¹⁰ is H, halogen. CN, CHO, or optionally substituted heteroaryl. In another aspect of this embodiment, R¹⁰ is H, halogen or CN. In another aspect of this embodiment, R¹⁰ is H. In another aspect of this embodiment, R¹⁰ is halogen. In another aspect of this embodiment, R⁸ is NR¹¹R¹². In another aspect of this embodiment, R⁸ is NH₂. In another aspect of this embodiment, R⁸ is OR¹¹. In another aspect of this embodiment, R⁸ is OH. In another aspect of this embodiment, R⁹ is H. In another aspect of this embodiment, R⁹ is NR¹¹R¹². In another aspect of this embodiment, R⁹ is NH₂. In another aspect of this embodiment, R^{a} is H, —C(═O)R¹¹ or —C(═O)OR¹¹. In another aspect of this embodiment, R^{a} is H. In another aspect of this embodiment, R⁷ is H, —C(═O)R¹¹, —C(═O)OR¹¹ or

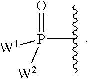

In another aspect of this embodiment, R⁷ is H. In another aspect of this embodiment, R⁷ is

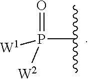

In one embodiment of Formulas I-II, R¹¹ or R¹² is independently H, (C₁-C₈)alkyl, (C₂-C₈)alkenyl, (C₂-C₈)alkynyl, (C₄-C₈)carbocyclylalkyl, optionally substituted aryl, optionally substituted heteroaryl, —C(═O)(C₁-C₈)alkyl, —S(O)ₙ(C₁-C₈)alkyl or aryl(C₁-C₈)alkyl. In another embodiment, R¹¹ and R¹² taken together with a nitrogen to which they are both attached, form a 3 to 7 membered heterocyclic ring wherein any one carbon atom of said heterocyclic ring can optionally be replaced with —O—, —S— or —NR^{a}—. Therefore, by way of example and not limitation, the moiety —NR¹¹R¹² can be represented by the heterocycles:

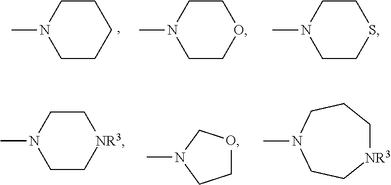

and the like.

In another embodiment of Formulas I-II, each R³, R⁵, R⁶, R¹¹ or R¹² is, independently, (C₁-C₈)alkyl, (C₂-C₈)alkenyl, (C₂-C₈)alkynyl or aryl(C₁-C₈)alkyl, wherein said (C₁-C₈)alkyl, (C₂-C₈)alkenyl, (C₂-C₈)alkynyl or aryl(C₁-C₈)alkyl are, independently, optionally substituted with one or more halo, hydroxy, CN, N₃, N(R^{a})₂ or OR^{a}. Therefore, by way of example and not limitation, R³, R⁴, R⁵, R⁶, R¹¹ or R¹² could represent moieties such as —CH(NH₂)CH₃, —CH(OH)CH2CH3, —CH(NH₂)CH(CH₃)₂, —CH₂CF₃, —(CH₂)₂CH(N₃)CH₃, —(CH₂)₆NH₂ and the like.

In another embodiment of Formula I-II, R³, R⁵, R⁶, R¹¹ or R¹² is (C₁-C₈)alkyl wherein one or more of the non-terminal carbon atoms of each said (C₁-C₈)alkyl may be optionally replaced with —O—, —S— or —NR^{a}—. Therefore, by way of example and not limitation, R³, R⁵, R⁶, R¹¹ or R¹² could represent moieties such as —CH₂OCH₃, —CH₂OCH₂CH₃, —CH₂OCH(CH₃)₂, —CH₂SCH₃, —(CH₂)₆OCH₃, —(CH₂)₆N(CH₃)₂ and the like.

In another embodiment, provided is a compound of Formulas I-II that is

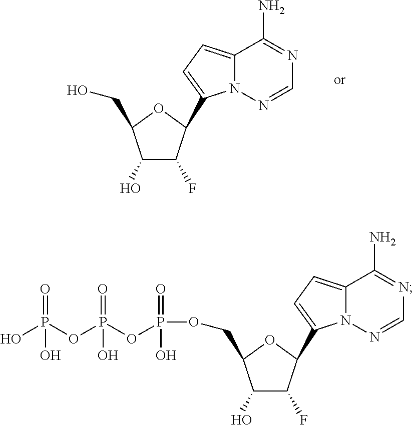 [SMILES: Nc1ncnn2c([C@@H]3O[C@H](CO)[C@@H](O)[C@H]3F)ccc12 ; Nc1ncnn2c([C@@H]3O[C@H](COP(=O)(O)OP(=O)(O)OP(=O)(O)O)[C@@H](O)[C@H]3F)ccc12]

pharmaceutically acceptable salt or ester thereof.

### DEFINITIONS

Unless stated otherwise, the following terms and phrases as used herein are intended to have the following meanings:

When trade names are used herein, applicants intend to independently include the tradename product and the active pharmaceutical ingredient(s) of the tradename product.

As used herein, “a compound of the invention” or “a compound of Formula I” means a compound of Formula I or a pharmaceutically acceptable salt, thereof. Similarly, with respect to isolatable intermediates, the phrase “a compound of Formula (number)” means a compound of that formula and pharmaceutically acceptable salts, thereof.

“Alkyl” is hydrocarbon containing normal, secondary, tertiary or cyclic carbon atoms. For example, an alkyl group can have 1 to 20 carbon atoms (i.e, C₁-C₂₀ alkyl), 1 to 8 carbon atoms (i.e., C₁-C₈ alkyl), or 1 to 6 carbon atoms (i.e., C₁-C₆ alkyl). Examples of suitable alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂C₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—CH(CH₃) CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, and octyl (—(CH₂)₇CH₃).

“Alkoxy” means a group having the formula —O-alkyl, in which an alkyl group, as defined above, is attached to the parent molecule via an oxygen atom. The alkyl portion of an alkoxy group can have 1 to 20 carbon atoms (i.e., C₁-C₂₀ alkoxy), 1 to 12 carbon atoms (i.e., C₁-C₁₂ alkoxy), or 1 to 6 carbon atoms (i.e., C₁-C₆ alkoxy). Examples of suitable alkoxy groups include, but are not limited to, methoxy (—O—CH₃ or —OMe), ethoxy (—OCH₂CH₃ or —OEt), t-butoxy (—O—C(CH₃)₃ or —OtBu) and the like.

“Haloalkyl” is an alkyl group, as defined above, in which one or more hydrogen atoms of the alkyl group is replaced with a halogen atom. The alkyl portion of a haloalkyl group can have 1 to 20 carbon atoms (i.e., C₁-C₂₀ haloalkyl), 1 to 12 carbon atoms (i.e., C₁-C₁₂ haloalkyl), or 1 to 6 carbon atoms (i.e., C₁-C₆ alkyl). Examples of suitable haloalkyl groups include, but are not limited to, —CF₃, —CHF₂, —CFH₂, —CH₂CF₃, and the like.

“Alkenyl” is a hydrocarbon containing normal, secondary, tertiary or cyclic carbon atoms with at least one site of unsaturation, i.e. a carbon-carbon, sp² double bond. For example, an alkenyl group can have 2 to 20 carbon atoms (i.e., C₂-C₂₀ alkenyl), 2 to 8 carbon atoms (i.e., C₂-C₈ alkenyl), or 2 to 6 carbon atoms (i.e., C₂-C₆ alkenyl). Examples of suitable alkenyl groups include, but are not limited to, ethylene or vinyl (—CH═CH₂), allyl (—CH₂CH═CH₂), cyclopentenyl (—C₅H₇), and 5-hexenyl (—CH₂CH₂CH₂CH₂CH═CH₂).

“Alkynyl” is a hydrocarbon containing normal, secondary, tertiary or cyclic carbon atoms with at least one site of unsaturation, i.e. a carbon-carbon, sp triple bond. For example, an alkynyl group can have 2 to 20 carbon atoms (i.e., C₂-C₂₀ alkynyl), 2 to 8 carbon atoms (i.e., C₂-C₈ alkyne,), or 2 to 6 carbon atoms (i.e., C₂-C₆ alkynyl). Examples of suitable alkynyl groups include, but are not limited to, acetylenic (—C≡CH), propargyl (—CH₂C≡CH), and the like.

“Alkylene” refers to a saturated, branched or straight chain or cyclic hydrocarbon radical having two monovalent radical centers derived by the removal of two hydrogen atoms from the same or two different carbon atoms of a parent alkane. For example, an alkylene group can have 1 to 20 carbon atoms, 1 to 10 carbon atoms, or 1 to 6 carbon atoms. Typical alkylene radicals include, but are not limited to, methylene (—CH₂—), 1,1-ethyl (—CH(CH₃)—), 1,2-ethyl (—CH₂CH₂—), 1,1-propyl (—CH(CH₂CH₃)—), 1,2-propyl (—CH₂CH(CH₃)—), 1,3-propyl (—CH₂CH₂CH₂—), 1,4-butyl (—CH₂CH₂CH₂CH₂—), and the like.

“Alkenylene” refers to an unsaturated, branched or straight chain or cyclic hydrocarbon radical having two monovalent radical centers derived by the removal of two hydrogen atoms from the same or two different carbon atoms of a parent alkene. For example, and alkenylene group can have 1 to 20 carbon atoms, 1 to 10 carbon atoms, or 1 to 6 carbon atoms. Typical alkenylene radicals include, but are not limited to, 1,2-ethylene (—CH═CH—).

“Alkynylene” refers to an unsaturated, branched or straight chain or cyclic hydrocarbon radical having two monovalent radical centers derived by the removal of two hydrogen atoms from the same or two different carbon atoms of a parent alkyne. For example, an alkynylene group can have 1 to 20 carbon atoms, 1 to 10 carbon atoms, or 1 to 6 carbon atoms. Typical alkynylene radicals include, but are not limited to, acetylene (—C≡C—), propargyl (—CH₂C≡C—), and 4-pentynyl (—CH₂CH₂CH₂C≡C—).

“Amino” refers generally to a nitrogen radical which can be considered a derivative of ammonia, having the formula —N(X)₂, where each “X” is independently H, substituted or unsubstituted alkyl, substituted or unsubstituted carbocyclyl, substituted or unsubstituted heterocyclyl, etc. The hybridization of the nitrogen is approximately sp³. Nonlimiting types of amino include —NH₂, —N(alkyl)₂, —NH(alkyl), —N(carbocyclyl)₂, —NH(carbocyclyl), —N(heterocyclyl)₂, —NH(heterocyclyl), —N(aryl)₂, —NH(aryl), —N(alkyl)(aryl), —N(alkyl)(heterocyclyl), —N(carbocyclyl)(heterocyclyl), —N(aryl(heteroaryl), —N(alkyl)(heteroaryl), etc. The term “alkylamino” refers to an amino group substituted with at least one alkyl group. Nonlimiting examples of amino groups include —NH₂, —NH(CH₃), —N(CH₃)₂, —NH(CH₂CH₃), —N(CH₂CH₃)₂, —NH(phenyl), —N(phenyl)₂, —NH(benzyl), —N(benzyl)₂, etc. Substituted alkylamino refers generally to alkylamino groups, as defined above, in which at least one substituted alkyl, as defined herein, is attached to the amino nitrogen atom. Non-limiting examples of substituted alkylamino includes —NH(alkylene-C(O)—OH), —NH(alkylene-C(O)—O-alkyl), —N(alkylene-C(O—OH)₂, —N(alkylene-C(O)—O-alkyl)₂, etc.

“Aryl” means an aromatic hydrocarbon radical derived by the removal of one hydrogen atom from a single carbon atom of a parent aromatic ring system. For example, an aryl group can have 6 to 20 carbon atoms, 6 to 14 carbon atoms, or 6 to 10 carbon atoms. Typical aryl groups include, but are not limited to, radicals derived from benzene (e.g., phenyl), substituted benzene, naphthalene, anthracene, biphenyl, and the like.

“Arylalkyl” refers to an acyclic alkyl radical in which one of the hydrogen atoms bonded to a carbon atom, typically a terminal or sp³ carbon atom, is replaced with an aryl radical. Typical arylalkyl groups include, but are not limited to, benzyl, 2-phenylethan-1-yl, naphthylmethyl, 2-naphthylethan-1-yl, naphthobenzyl, 2-naphthophenylethan-1-yl and the like. The arylalkyl group can comprise 7 to 20 carbon atoms, e.g., the alkyl moiety is 1 to 6 carbon atoms and the aryl moiety is 6 to 14 carbon atoms.

“Arylalkenyl” refers to an acyclic alkenyl radical in which one of the hydrogen atoms bonded to a carbon atom, typically a terminal or sp³ carbon atom, but also an sp² carbon atom, is replaced with an aryl radical. The aryl portion of the arylalkenyl can include, for example, any of the aryl groups disclosed herein, and the alkenyl portion of the arylalkenyl can include, for example, any of the alkenyl groups disclosed herein. The arylalkenyl group can comprise 8 to 20 carbon atoms, e.g., the alkenyl moiety is 2 to 6 carbon atoms and the aryl moiety is 6 to 14 carbon atoms.

“Arylalkynyl” refers to an acyclic alkynyl radical in which one of the hydrogen atoms bonded to a carbon atom, typically a terminal or sp³ carbon atom, but also an sp carbon atom, is replaced with an aryl radical. The aryl portion of the arylalkynyl can include, for example, any of the aryl groups disclosed herein, and the alkynyl portion of the arylalkynyl can include, for example, any of the alkynyl groups disclosed herein. The arylalkynyl group can comprise 8 to 20 carbon atoms, e.g., the alkynyl moiety is 2 to 6 carbon atoms and the aryl moiety is 6 to 14 carbon atoms.

The term “substituted” in reference to alkyl, alkylene, aryl, arylalkyl, alkoxy, heterocyclyl, heteroaryl, carbocyclyl, etc., for example, “substituted alkyl”, “substituted alkylene”, “substituted aryl”, “substituted arylalkyl”, “substituted heterocyclyl”, and “substituted carbocyclyl” means alkyl, alkylene, aryl, arylalkyl, heterocyclyl, carbocyclyl respectively, in which one or more hydrogen atoms are each independently replaced with a non-hydrogen substituent. Typical substituents include, but are not limited to, —X, R, —R^{b}, —O⁻, —OR^{b}, —SR^{b}, —S⁻, —NR^{b}₂, —N⁺R^{b}₃, ═NR^{b}, —CX₃, —CN, —OCN, —SCN, —N═C═O, —NCS, —NO, —NO₂, ═N₂, —N₃, —NHC(═O)R^{b}, —OC(═O)R^{b}, —NHC(═O)NR^{b}₂, —S(═O)₂—, —S(═O)₂OH, —S(═O)₂R^{b}, —OS(═O)₂OR^{b}, —S(═O)₂NR^{b}₂, —S(═O)R^{b}, —OP(═O)(OR^{b})₂, —P(═O)(OR^{b})₂, —P(═O)(O⁻)₂, —P(═O)(OH)₂, —P(O)(OR^{b})(O⁻), —C(═O)R^{b}, —C(═O)X, —C(S)R^{b}, —C(O)OR^{b}, —C(O)O⁻, —C(S)OR^{b}, —C(O)SR^{b}, —C(S)SR^{b}, —C(O)NR^{b}₂, —C(S)NR^{b}₂, —C(═NR^{b})NR^{b}₂, where each X is independently a halogen; F, Cl, Br, or I; and each R^{b} is independently H, alkyl, aryl, arylalkyl, a heterocycle, or a protecting group or prodrug moiety. Alkylene, alkenylene, and alkynylene groups may also be similarly substituted. Unless otherwise indicated, when the term “substituted” is used in conjunction with groups such as arylalkyl, which have two or more moieties capable of substitution, the substituents can be attached to the aryl moiety, the alkyl moiety, or both.

The term “prodrug” as used herein refers to any compound that when administered to a biological system generates the drug substance, i.e., active ingredient, as a result of spontaneous chemical reaction(s), enzyme catalyzed chemical reaction(s), photolysis, and/or metabolic chemical reaction(s). A prodrug is thus a covalently modified analog or latent form of a therapeutically active compound.

One skilled in the art will recognize that substituents and other moieties of the compounds of Formula I-II should be selected in order to provide a compound which is sufficiently stable to provide a pharmaceutically useful compound which can be formulated into an acceptably stable pharmaceutical composition. Compounds of Formula I-II which have such stability are contemplated as falling within the scope of the present invention.

“Heteroalkyl” refers to an alkyl group where one or more carbon atoms have been replaced with a heteroatom, such as, O, N, or S. For example, if the carbon atom of the alkyl group which is attached to the parent molecule is replaced with a heteroatom (e.g., O, N, or S) the resulting heteroalkyl groups are, respectively, an alkoxy group (e.g., —OCH₃, etc.), an amine (e.g., —NHCH₃, —N(CH₃)₂, etc.), or a thioalkyl group (e.g., —SCH₃). If a non-terminal carbon atom of the alkyl group which is not attached to the parent molecule is replaced with a heteroatom (e.g., O, N, or S) the resulting heteroalkyl groups are, respectively, an alkyl ether (e.g., —CH₂CH₂—O—CH₃, etc.), an alkyl amine (e.g., —CH₂NHCH₃, —CH₂N(CH₃)₂, etc.), or a thioalkyl ether (e.g., —CH₂—S—CH₃). If a terminal carbon atom of the alkyl group is replaced with a heteroatom (e.g., O, N, or S), the resulting heteroalkyl groups are, respectively, a hydroxyalkyl group (e.g., —CH₂CH₂—OH), an aminoalkyl group (e.g., —CH₂NH₂), or an alkyl thiol group (e.g., —CH₂CH₂—SH). A heteroalkyl group can have, for example, 1 to 20 carbon atoms, 1 to 10 carbon atoms, or 1 to 6 carbon atoms. A C₁-C₆ heteroalkyl group means a heteroalkyl group having 1 to 6 carbon atoms.

“Heterocycle” or “heterocyclyl” as used herein includes by way of example and not limitation those heterocycles described in Paquette. Leo A.; Principles of Modern Heterocyclic Chemistry (W. A. Benjamin, New York, 1968), particularly Chapters 1, 3, 4, 6, 7, and 9; The Chemistry of Heterocyclic Compounds. A Series of Monographs” (John Wiley & Sons, New York. 1950 to present), in particular Volumes 13, 14, 16, 19, and 28; and J. Am. Chem. Soc. (1960) 82:5566. In one specific embodiment of the invention “heterocycle” includes a “carbocycle” as defined herein, wherein one or more (e.g. 1, 2, 3, or 4) carbon atoms have been replaced with a heteroatom (e.g. O, N, or S). The terms “heterocycle” or “heterocyclyl” includes saturated rings, partially unsaturated rings, and aromatic rings (i.e., heteroaromatic rings). Substituted heterocyclyls include, for example, heterocyclic rings substituted with any of the substituents disclosed herein including carbonyl groups. A non-limiting example of a carbonyl substituted heterocyclyl is:

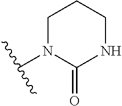 [SMILES: *N1CCCNC1=O]

Examples of heterocycles include by way of example and not limitation pyridyl, dihydroypyridyl, tetrahydropyridyl (piperidyl), thiazolyl, tetrahydrothiophenyl, sulfur oxidized tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, piperidinyl, 4-piperidonyl, pyrrolidinyl, 2-pyrrolidonyl, pyrrolinyl, tetrahydrofuranyl, tetrahydroquinolinyl, tetrahydroisoquinolinyl, decahydroquinolinyl, octahydroisoquinolinyl, azocinyl, triazinyl, 6H-1,2,5-thiadiazinyl, 2H,6H-1,5,2-dithiazinyl, thienyl, thianthrenyl, pyranyl, isobenzofuranyl, chromenyl, xanthenyl, phenoxathinyl, 2H-pyrrolyl, isothiazolyl, isoxazolyl pyrazinyl, pyridazinyl, indolizinyl, isoindolyl, 3H-indolyl, 1H-indazoly, purinyl, 4H-quinolizinyl, phthalazinyl, naphthyridinyl, quinoxalinyl, quinazolinyl, cinnolinyl, pteridinyl, 4aH-carbazolyl, carbazolyl, β-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, piperazinyl, indolinyl, isoindolinyl, quinuclidinyl, morpholinyl, oxazolidinyl, benzotriazolyl, benzisoxazolyl, oxindolyl, benzoxazolinyl, isatinoyl, and bis-tetrahydrofuranyl:

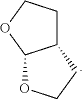

By way of example and not limitation, carbon bonded heterocycles are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline. Still more typically, carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl.

By way of example and not limitation, nitrogen bonded heterocycles are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or β-carboline. Still more typically, nitrogen bonded heterocycles include 1-aziridyl, 1-azetedyl, 1-pyrrolyl, 1-imidazolyl, 1-pyrazolyl, and 1-piperidinyl.

“Heterocyclylalkyl” refers to an acyclic alkyl radical in which one of the hydrogen atoms bonded to a carbon atom, typically a terminal or sp³ carbon atom, is replaced with a heterocyclyl radical (i.e., a heterocyclyl-alkylene-moiety). Typical heterocyclyl alkyl groups include, but are not limited to heterocyclyl-CH₂—, 2-(heterocyclyl)ethan-1-yl, and the like, wherein the “heterocyclyl” portion includes any of the heterocyclyl groups described above, including those described in Principles of Modern Heterocyclic Chemistry. One skilled in the art will also understand that the heterocyclyl group can be attached to the alkyl portion of the heterocyclyl alkyl by means of a carbon-carbon bond or a carbon-heteroatom bond, with the proviso that the resulting group is chemically stable. The heterocyclyl alkyl group comprises 3 to 20 carbon atoms, e.g., the alkyl portion of the arylalkyl group is 1 to 6 carbon atoms and the heterocyclyl moiety is 2 to 14 carbon atoms. Examples of heterocyclylalkyls include by way of example and not limitation 5-membered sulfur, oxygen, and/or nitrogen containing heterocycles such as thiazolylmethyl, 2-thiazolylethan-1-yl, imidazolylmethyl, oxazolylmethyl, thiadiazolylmethyl, etc., 6-membered sulfur, oxygen, and/or nitrogen containing heterocycles such as piperidinylmethyl, piperazinylmethyl, morpholinylmethyl, pyridinylmethyl, pyridizylmethyl, pyrimidylmethyl, pyrazinylmethyl, etc.

“Heterocyclylalkenyl” refers to an acyclic alkenyl radical in which one of the hydrogen atoms bonded to a carbon atom, typically a terminal or sp³ carbon atom, but also a sp² carbon atom, is replaced with a heterocyclyl radical (i.e., a heterocyclyl-alkenylene-moiety). The heterocyclyl portion of the heterocyclyl alkenyl group includes any of the heterocyclyl groups described herein, including those described in Principles of Modern Heterocyclic Chemistry, and the alkenyl portion of the heterocyclyl alkenyl group includes any of the alkenyl groups disclosed herein. One skilled in the art will also understand that the heterocyclyl group can be attached to the alkenyl portion of the heterocyclyl alkenyl by means of a carbon-carbon bond or a carbon-heteroatom bond, with the proviso that the resulting group is chemically stable. The heterocyclyl alkenyl group comprises 4 to 20 carbon atoms, e.g., the alkenyl portion of the heterocyclyl alkenyl group is 2 to 6 carbon atoms and the heterocyclyl moiety is 2 to 14 carbon atoms.

“Heterocyclylalkynyl” refers to an acyclic alkynyl radical in which one of the hydrogen atoms bonded to a carbon atom, typically a terminal or sp³ carbon atom, but also an sp carbon atom, is replaced with a heterocyclyl radical (i.e., a heterocyclyl-alkynylene-moiety). The heterocyclyl portion of the heterocyclyl alkynyl group includes any of the heterocyclyl groups described herein, including those described in Principles of Modern Heterocyclic Chemistry, and the alkynyl portion of the heterocyclyl alkynyl group includes any of the alkynyl groups disclosed herein. One skilled in the art will also understand that the heterocyclyl group can be attached to the alkynyl portion of the heterocyclyl alkynyl by means of a carbon-carbon bond or a carbon-heteroatom bond, with the proviso that the resulting group is chemically stable. The heterocyclyl alkynyl group comprises 4 to 20 carbon atoms, e.g., the alkynyl portion of the heterocyclyl alkynyl group is 2 to 6 carbon atoms and the heterocyclyl moiety is 2 to 14 carbon atoms.

“Heteroaryl” refers to an aromatic heterocyclyl having at least one heteroatom in the ring. Non-limiting examples of suitable heteroatoms which can be included in the aromatic ring include oxygen, sulfur, and nitrogen. Non-limiting examples of heteroaryl rings include all of those aromatic rings listed in the definition of “heterocyclyl”, including pyridinyl, pyrrolyl, oxazolyl, indolyl, isoindolyl, purinyl, furanyl, thienyl, benzofuranyl, benzothiophenyl, carbazolyl, imidazolyl, thiazolyl, isoxazolyl, pyrazolyl, isothiazolyl, quinolyl, isoquinolyl, pyridazyl, pyrimidyl, pyrazyl, etc.

“Carbocycle” or “carbocyclyl” refers to a saturated (i.e., cycloalkyl), partially unsaturated (e.g., cycloakenyl, cycloalkadienyl, etc.) or aromatic ring having 3 to 7 carbon atoms as a monocycle, 7 to 12 carbon atoms as a bicycle, and up to about 20 carbon atoms as a polycycle. Monocyclic carbocycles have 3 to 7 ring atoms, still more typically 5 or 6 ring atoms. Bicyclic carbocycles have 7 to 12 ring atoms, e.g., arranged as a bicyclo[4,5], [5,5], [5,6] or [6,6] system, or 9 or 10 ring atoms arranged as a bicyclo[5.6] or [6,6] system, or spiro-fused rings. Non-limiting examples of monocyclic carbocycles include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, and phenyl. Non-limiting examples of bicyclo carbocycles includes naphthyl, tetrahydronapthalene, and decaline.

“Carbocyclylalkyl” refers to an acyclic alkyl radical in which one of the hydrogen atoms bonded to a carbon atom is replaced with a carbocyclyl radical as described herein. Typical, but non-limiting, examples of carbocyclylalkyl groups include cyclopropylmethyl, cyclopropylethyl, cyclobutylmethyl, cyclopentylmethyl and cyclohexylmethyl.

“Arylheteroalkyl” refers to a heteroalkyl as defined herein, in which a hydrogen atom (which may be attached either to a carbon atom or a heteroatom) has been replaced with an aryl group as defined herein. The aryl groups may be bonded to a carbon atom of the heteroalkyl group, or to a heteroatom of the heteroalkyl group, provided that the resulting arylheteroalkyl group provides a chemically stable moiety. For example, an arylheteroalkyl group can have the general formulae -alkylene-O-aryl, -alkylene-O-alkylene-aryl, -alkylene-NH-aryl, -alkylene-NH-alkylene-aryl, -alkylene-S-aryl, -alkylene-S-alkylene-aryl, etc. In addition, any of the alkylene moieties in the general formulae above can be further substituted with any of the substituents defined or exemplified herein.

“Heteroarylalkyl” refers to an alkyl group, as defined herein, in which a hydrogen atom has been replaced with a heteroaryl group as defined herein. Non-limiting examples of heteroaryl alkyl include —CH₂-pyridinyl, —CH₂-pyrrolyl, —CH₂-oxazolyl, —CH₂-indolyl, —CH₂-isoindolyl, —CH₂-purinyl, —CH₂-furanyl, —CH₂-thienyl, —CH₂-benzofuranyl, —CH₂-benzothiophenyl, —CH₂-carbazolyl, —CH₂-imidazolyl, —CH₂-thiazolyl, —CH₂-isoxazolyl, —CH₂-pyrazolyl, —CH₂-isothiazolyl, —CH₂-quinolyl, —CH₂-isoquinolyl, —CH₂-pyridazyl, —CH₂-pyrimidyl, —CH₂-pyrazyl, —CH(CH₃)-pyridinyl, —CH(CH₃)-pyrrolyl, —CH(CH₃)-oxazolyl, —CH(CH₃)-indolyl, —CH(CH₃)-isoindolyl, —CH(CH₃)-purinyl, —CH(CH₃)-furanyl, —CH(CH₃)-thienyl, —CH(CH₃)-benzofuranyl, —CH(CH₃)-benzothiophenyl, —CH(CH₃)-carbazolyl, —CH(CH₃)-imidazolyl, —CH(CH₃)-thiazolyl, —CH(CH₃)-isoxazolyl, —CH(CH₃)-pyrazolyl. —CH(CH₃)-isothiazolyl, —CH(CH₃)-quinolyl, —CH(CH₃)-isoquinolyl, —CH(CH₃)-pyridazyl, —CH(CH₃)-pyrimidyl, —CH(CH₃)-pyrazyl, etc.

The term “optionally substituted” in reference to a particular moiety of the compound of Formula I-II (e.g., an optionally substituted aryl group) refers to a moiety wherein all substituents are hydrogen or wherein one or more of the hydrogens of the moiety may be replaced by substituents such as those listed under the definition of “substituted”.

The term “optionally replaced” in reference to a particular moiety of the compound of Formula I-II (e.g., the carbon atoms of said (C₁-C₈)alkyl may be optionally replaced by —O—, —S—, or —NR^{a}—) means that one or more of the methylene groups of the (C₁-C₈)alkyl may be replaced by 0, 1, 2, or more of the groups specified (e.g., —O—, —S—, or —NR^{a}—).

The term “non-terminal carbon atom(s)” in reference to an alkyl, alkenyl, alkynyl, alkylene, alkenylene, or alkynylene moiety refers to the carbon atoms in the moiety that intervene between the first carbon atom of the moiety and the last carbon atom in the moiety. Therefore, by way of example and not limitation, in the alkyl moiety —CH₂(C*)H₂(C*)H₂CH₃ or alkylene moiety —CH₂(C*)H₂(C*)H₂CH₂— the C* atoms would be considered to be the non-terminal carbon atoms.

Certain Y and Y¹ alternatives are nitrogen oxides such as ⁺N(O)(R) or ⁺N(O)(OR). These nitrogen oxides, as shown here attached to a carbon atom, can also be represented by charge separated groups such as

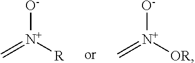

respectively, and are intended to be equivalent to the aforementioned representations for the purposes of describing this invention.

“Linker” or “link” means a chemical moiety comprising a covalent bond or a chain of atoms. Linkers include repeating units of alkyloxy (e.g. polyethyleneoxy, PEG, polymethyleneoxy) and alkylamino (e.g. polyethyleneamino, Jeffamine™); and diacid ester and amides including succinate, succinamide, diglycolate, malonate, and caproamide.

The terms such as “oxygen-linked”, “nitrogen-linked”, “carbon-linked”, “sulfur-linked”, or “phosphorous-linked” mean that if a bond between two moieties can be formed by using more than one type of atom in a moiety, then the bond formed between the moieties is through the atom specified. For example, a nitrogen-linked amino acid would be bonded through a nitrogen atom of the amino acid rather than through an oxygen or carbon atom of the amino acid.

Unless otherwise specified, the carbon atoms of the compounds of Formula I-II are intended to have a valence of four. In some chemical structure representations where carbon atoms do not have a sufficient number of variables attached to produce a valence of four, the remaining carbon substitutents needed to provide a valence of four should be assumed to be hydrogen. For example.

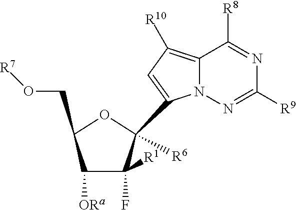

has the same meaning as

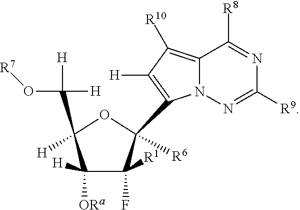

“Protecting group” refers to a moiety of a compound that masks or alters the properties of a functional group or the properties of the compound as a whole. The chemical substructure of a protecting group varies widely. One function of a protecting group is to serve as an intermediate in the synthesis of the parental drug substance. Chemical protecting groups and strategies for protection/deprotection are well known in the art. See: “Protective Groups in Organic Chemistry”, Theodora W. Greene (John Wiley & Sons, Inc., New York, 1991. Protecting groups are often utilized to mask the reactivity of certain functional groups, to assist in the efficiency of desired chemical reactions, e.g. making and breaking chemical bonds in an ordered and planned fashion. Protection of functional groups of a compound alters other physical properties besides the reactivity of the protected functional group, such as the polarity, lipophilicity (hydrophobicity), and other properties which can be measured by common analytical tools. Chemically protected intermediates may themselves be biologically active or inactive.

Protected compounds may also exhibit altered, and in some cases, optimized properties in vitro and in vivo, such as passage through cellular membranes and resistance to enzymatic degradation or sequestration. In this role, protected compounds with intended therapeutic effects may be referred to as prodrugs. Another function of a protecting group is to convert the parental drug into a prodrug, whereby the parental drug is released upon conversion of the prodrug in vivo. Because active prodrugs may be absorbed more effectively than the parental drug, prodrugs may possess greater potency in vivo than the parental drug. Protecting groups are removed either in vitro, in the instance of chemical intermediates, or in vivo, in the case of prodrugs. With chemical intermediates, it is not particularly important that the resulting products after deprotection, e.g. alcohols, be physiologically acceptable, although in general it is more desirable if the products are pharmacologically innocuous.

“Prodrug moiety” means a labile functional group which separates from the active inhibitory compound during metabolism, systemically, inside a cell, by hydrolysis, enzymatic cleavage, or by some other process (Bundgaard, Hans, “Design and Application of Prodrugs” in Textbook of Drug Design and Development (1991), P. Krogsgaard-Larsen and H. Bundgaard, Eds. Harwood Academic Publishers, pp. 113-191). Enzymes which are capable of an enzymatic activation mechanism with the phosphonate prodrug compounds of the invention include, but are not limited to, amidases, esterases, microbial enzymes, phospholipases, cholinesterases, and phosphases. Prodrug moieties can serve to enhance solubility, absorption and lipophilicity to optimize drug delivery, bioavailability and efficacy.

A prodrug moiety may include an active metabolite or drug itself.

Exemplary prodrug moieties include the hydrolytically sensitive or labile acyloxymethyl esters —CH₂OC(═O)R³⁰ and acyloxymethyl carbonates —CH₂OC(═O)OR³⁰ where R³⁰ is C₁-C₆ alkyl, C₁-C₆ substituted alkyl, C₆-C₂₀ aryl or C₆-C₂₀ substituted aryl. The acyloxyalkyl ester was used as a prodrug strategy for carboxylic acids and then applied to phosphates and phosphonates by Farquhar et al (1983) J. Pharm. Sci. 72: 324; also U.S. Pat. Nos. 4,816,570, 4,968,788, 5,663,159 and 5,792,756. In certain compounds of the invention, a prodrug moiety is part of a phosphate group. The acyloxyalkyl ester may be used to deliver phosphoric acids across cell membranes and to enhance oral bioavailability. A close variant of the acyloxyalkyl ester, the alkoxycarbonyloxyalkyl ester (carbonate), may also enhance oral bioavailability as a prodrug moiety in the compounds of the combinations of the invention. An exemplary acyloxymethyl ester is pivaloyloxymethoxy, (POM) —CH₂OC(═O)C(CH₃)₃. An exemplary acyloxymethyl carbonate prodrug moiety is pivaloyloxymethylcarbonate (POC) —CH₂OC(═O)OC(CH₃)₃.

The phosphate group may be a phosphate prodrug moiety. The prodrug moiety may be sensitive to hydrolysis, such as, but not limited to those comprising a pivaloyloxymethyl carbonate (POC) or POM group. Alternatively, the prodrug moiety may be sensitive to enzymatic potentiated cleavage, such as a lactate ester or a phosphonamidate-ester group.

Aryl esters of phosphorus groups, especially phenyl esters, are reported to enhance oral bioavailability (DeLambert et al (1994) J. Med. Chem, 37: 498). Phenyl esters containing a carboxylic ester ortho to the phosphate have also been described (Khamnei and Torrence, (1996) J. Med. Chem, 39:4109-4115). Benzyl esters are reported to generate the parent phosphonic acid. In some cases, substituents at the ortho or para-position may accelerate the hydrolysis. Benzyl analogs with an acylated phenol or an alkylated phenol may generate the phenolic compound through the action of enzymes, e.g. esterases, oxidases, etc., which in turn undergoes cleavage at the benzylic C—O bond to generate the phosphoric acid and the quinone methide intermediate. Examples of this class of prodrugs are described by Mitchell et al (1992) J. Chem. Soc. Perkin Trans. 12345; Brook et al WO 91/19721. Still other benzylic prodrugs have been described containing a carboxylic ester-containing group attached to the benzylic methylene (Glazier et al WO 91/19721). Thio-containing prodrugs are reported to be useful for the intracellular delivery of phosphonate drugs. These proesters contain an ethylthio group in which the thiol group is either esterified with an acyl group or combined with another thiol group to form a disulfide. Deesteritication or reduction of the disulfide generates the free thio intermediate which subsequently breaks down to the phosphoric acid and episulfide (Puech et al (1993) Antiviral Res., 22: 155-174; Benzaria et al (1996) J. Med. Chem, 39: 4958). Cyclic phosphonate esters have also been described as prodrugs of phosphorus-containing compounds (Erion et al, U.S. Pat. No. 6,312,662).

It is to be noted that all enantiomers, diastereomers, and racemic mixtures, tautomers, polymorphs, pseudopolymorphs of compounds within the scope of Formula I or Formula II and pharmaceutically acceptable salts thereof are embraced by the present invention. All mixtures of such enantiomers and diastereomers are within the scope of the present invention.

A compound of Formula I-II and its pharmaceutically acceptable salts may exist as different polymorphs or pseudopolymorphs. As used herein, crystalline polymorphism means the ability of a crystalline compound to exist in different crystal structures. The crystalline polymorphism may result from differences in crystal packing (packing polymorphism) or differences in packing between different conformers of the same molecule (conformational polymorphism). As used herein, crystalline pseudopolymorphism means the ability of a hydrate or solvate of a compound to exist in different crystal structures. The pseudopolymorphs of the instant invention may exist due to differences in crystal packing (packing pseudopolymorphism) or due to differences in packing between different conformers of the same molecule (conformational pseudopolymorphism). The instant invention comprises all polymorphs and pseudopolymorphs of the compounds of Formula I-II and their pharmaceutically acceptable salts.

A compound of Formula I-II and its pharmaceutically acceptable salts may also exist as an amorphous solid. As used herein, an amorphous solid is a solid in which there is no long-range order of the positions of the atoms in the solid. This definition applies as well when the crystal size is two nanometers or less. Additives, including solvents, may be used to create the amorphous forms of the instant invention. The instant invention comprises all amorphous forms of the compounds of Formula I-II and their pharmaceutically acceptable salts.

Selected substituents comprising the compounds of Formula I-II are present to a recursive degree. In this context, “recursive substituent” means that a substituent may recite another instance of itself. Because of the recursive nature of such substituents, theoretically, a large number of compounds may be present in any given embodiment. For example, R^{x} comprises a R^{y} substituent. R^{y} can be R. R can be W³. W³ can be W⁴ and W⁴ can be R or comprise substituents comprising R^{y}. One of ordinary skill in the art of medicinal chemistry understands that the total number of such substituents is reasonably limited by the desired properties of the compound intended. Such properties include, by way of example and not limitation, physical properties such as molecular weight, solubility or log P, application properties such as activity against the intended target, and practical properties such as ease of synthesis.

By way of example and not limitation, W³ and R^{y} are recursive substituents in certain embodiments. Typically, each recursive substituent can independently occur 20, 19, 18, 17, 16, 15, 14, 13, 12, 11, 10, 9, 8, 7, 6, 5, 4, 3, 2, 1, or 0, times in a given embodiment. More typically, each recursive substituent can independently occur 12 or fewer times in a given embodiment. Even more typically, each recursive substituent can independently occur 3 or fewer times in a given embodiment. For example, W³ will occur 0 to 8 times, R^{y} will occur 0 to 6 times in a given embodiment. Even more typically, W³ will occur 0 to 6 times and R^{y} will occur 0 to 4 times in a given embodiment.

Recursive substituents are an intended aspect of the invention. One of ordinary skill in the art of medicinal chemistry understands the versatility of such substituents. To the degree that recursive substituents are present in an embodiment of the invention, the total number will be determined as set forth above.

The modifier “about” used in connection with a quantity is inclusive of the stated value and has the meaning dictated by the context (e.g., includes the degree of error associated with measurement of the particular quantity).

The compounds of the Formula I-II may comprise a phosphate group as R⁷, which may be a prodrug moiety

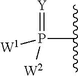

wherein each Y or Y¹ is, independently, O, S, NR, ⁺N(O)(R), N(OR), ⁺N(O)(OR), or N—NR₂; W¹ and W², when taken together, are —Y³(C(R^{y})₂)₃Y³—; or one of W¹ or W² together with either R³ or R⁴ is —Y³— and the other of W¹ or W² is Formula Ia; or W¹ and W² are each, independently, a group of Formula Ia:

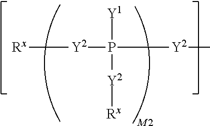

wherein:

each Y² is independently a bond, O, CR₂, NR, ⁺N(O)(R), N(OR), ⁺N(O)(OR), N—NR₂, S, S—S, S(O), or S(O)₂;

each Y³ is independently O, S, or NR;

M2 is 0, 1 or 2;

each R^{y} is independently H, F, Cl, Br, I, OH. R, —C(═Y¹)R, —C(═Y¹)OR, —C(═Y¹)N(R)₂, —N(R)₂, —⁺N(R)₃, —SR, —S(O)R, —S(O)₂R, —S(O)(OR), —S(O)₂(OR), —OC(═Y¹)R, —OC(═Y¹)OR, —OC(═Y¹)(N(R)₂), —SC(═Y¹)R, —SC(═Y¹)OR, —SC(═Y¹)(N(R)₂), —N(R)C(═Y¹)R, —N(R)C(═Y¹)OR, or —N(R)C(═Y¹)N(R)₂, —SO₂NR₂, —CN, —N₃, —NO₂, —OR, a protecting group or W³; or when taken together, two R^{y} on the same carbon atom form a carbocyclic ring of 3 to 7 carbon atoms;

each R^{x} is independently R^{y}, a protecting group, or the formula:

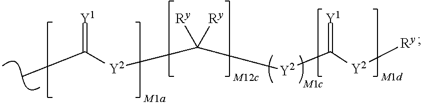

wherein:

M1a, M1c, and M1d are independently 0 or 1:

M12c is 0, 1, 2, 3, 4, 5, 6, 7, 8, 9, 10, 11 or 12;

each R is H, halogen, (C₁-C₈) alkyl, (C₁-C₈) substituted alkyl, (C₂-C₈) alkenyl, (C₂-C₈) substituted alkenyl, (C₂-C₈) alkynyl, (C₂-C₈) substituted alkynyl, C₆-C₂₀, aryl, C₆-C₂₀ substituted aryl, C₂-C₂₀ heterocycle, C₂-C₂₀ substituted heterocyclyl, arylalkyl, substituted arylalkyl or a protecting group;

W³ is W⁴ or W⁵; W⁴ is R, —C(Y¹)R^{y}, —C(Y¹)W⁵, —SO₂R^{y}, or —SO₂W⁵; and W⁵ is a carbocycle or a heterocycle wherein W⁵ is independently substituted with 0 to 3 R^{y} groups.

W⁵ carbocycles and W⁵ heterocycles may be independently substituted with 0 to 3 R^{y} groups. W⁵ may be a saturated, unsaturated or aromatic ring comprising a mono- or bicyclic carbocycle or heterocycle. W⁵ may have 3 to 10 ring atoms, e.g., 3 to 7 ring atoms. The W⁵ rings are saturated when containing 3 ring atoms, saturated or mono-unsaturated when containing 4 ring atoms, saturated, or mono- or di-unsaturated when containing 5 ring atoms, and saturated, mono- or di-unsaturated, or aromatic when containing 6 ring atoms.

A W⁵ heterocycle may be a monocycle having 3 to 7 ring members (2 to 6 carbon atoms and 1 to 3 heteroatoms selected from N, O, P, and S) or a bicycle having 7 to 10 ring members (4 to 9 carbon atoms and 1 to 3 heteroatoms selected from N, O, P, and S). W⁵ heterocyclic monocycles may have 3 to 6 ring atoms (2 to 5 carbon atoms and 1 to 2 heteroatoms selected from N, O, and S); or 5 or 6 ring atoms (3 to 5 carbon atoms and 1 to 2 heteroatoms selected from N and S). W⁵ heterocyclic bicycles have 7 to 10 ring atoms (6 to 9 carbon atoms and 1 to 2 heteroatoms selected from N, O, and S) arranged as a bicyclo[4,5], [5,5], [5,6], or [6,6] system; or 9 to 10 ring atoms (8 to 9 carbon atoms and 1 to 2 hetero atoms selected from N and S) arranged as a bicyclo[5.6] or [6,6] system. The W⁵ heterocycle may be bonded to Y² through a carbon, nitrogen, sulfur or other atom by a stable covalent bond.

W⁵ heterocycles include for example, pyridyl, dihydropyridyl isomers, piperidine, pyridazinyl, pyrimidinyl, pyrazinyl, s-triazinyl, oxazolyl, imidazolyl, thiazolyl, isoxazolyl, pyrazolyl, isothiazolyl, furanyl, thiofuranyl, thienyl, and pyrrolyl. W⁵ also includes, but is not limited to, examples such as:

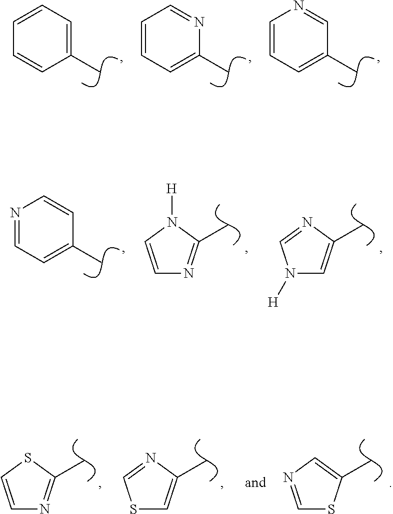

W⁵ carbocycles and heterocycles may be independently substituted with 0 to 3 R groups, as defined above. For example, substituted W⁵ carbocycles include:

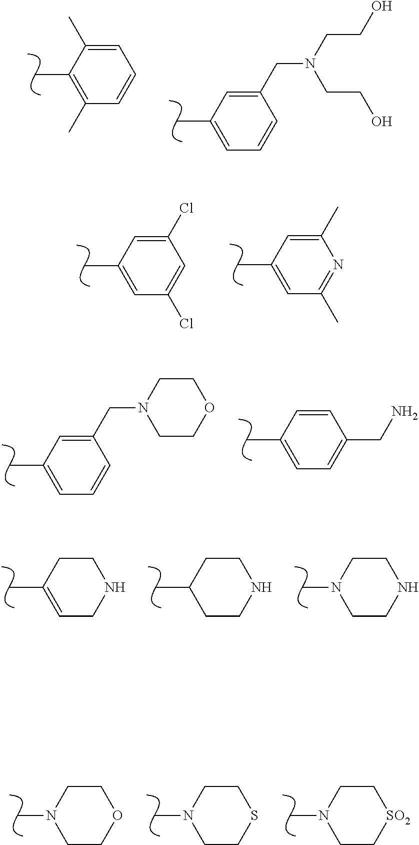

Examples of substituted phenyl carbocycles include:

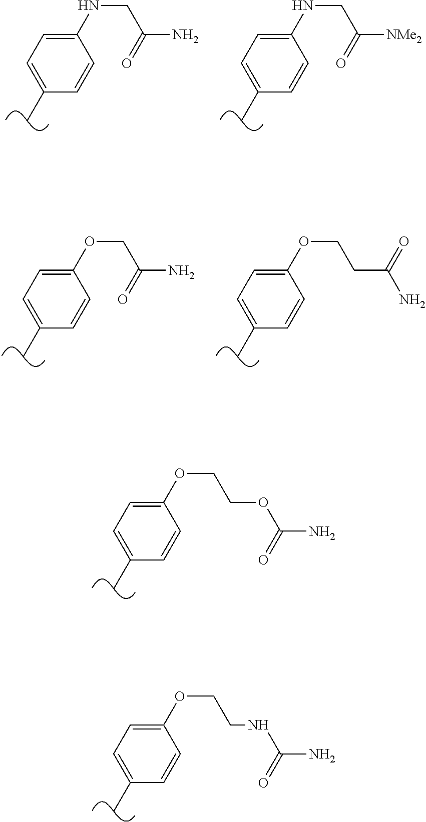

Embodiments of

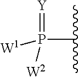

of Formula I-II compounds include substructures such as:

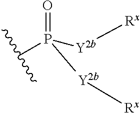

wherein each Y^{2b} is, independently, O or N(R). In another aspect of this embodiment, each Y^{2b} is O and each R^{x} is independently:

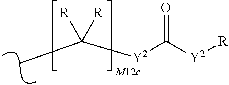

wherein M12c is 1, 2 or 3 and each Y² is independently a bond, O, CR₂, or S. In another aspect of this embodiment, one Y^{2b}—R^{x} is NH(R) and the other Y^{2b}—R^{x} is O—R^{x} wherein R^{x} is:

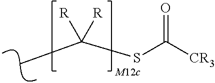

wherein M12c is 2. In another aspect of this embodiment, each Y^{2b} is O and each R^{x} is independently:

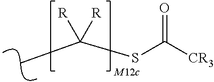

wherein M12c is 2. In another aspect of this embodiment, each Y^{2b} is O and each R^{x} is independently:

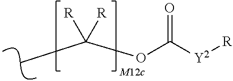

wherein M12c is 1 and Y² is a bond, O, or CR₂.

Other embodiments of

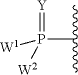

of Formulas I-III compounds include substructures such as:

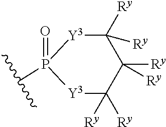

wherein each Y³ is, independently, O or N(R). In another aspect of this embodiment, each Y³ is O. In another aspect of this embodiment, the substructure is:

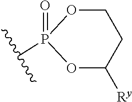

wherein R^{y} is W⁵ as defined herein.

Another embodiment of

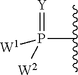

of Formula I-II includes the substructures:

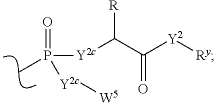

wherein each Y^{2c} is, independently, O, N(R^{y}) or S.

Another embodiment of

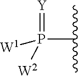

of Formula I-II compounds includes the substructures wherein one of W¹ or W² together with either R³ is —Y³— and the other of W¹ or W² is Formula Ia. Such an embodiment is represented by a compound of Formula Ib selected from:

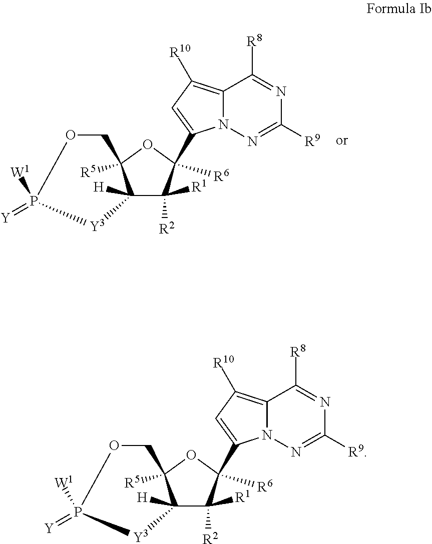

In another aspect of the embodiment of Formula Ib, each Y and Y³ is O. In another aspect of the embodiment of Formula Ib, W¹ or W² is Y^{2b}—R^{x}; each Y, Y³ and Y^{2b} is O and R^{x} is:

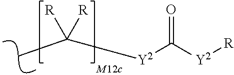

wherein M12c is 1, 2 or 3 and each Y is independently a bond, O, CR₂, or S. In another aspect of the embodiment of Formula Ib, W¹ or W² is Y^{2b}—R^{x}; each Y, Y³ and Y^{2b} is O and R^{x} is:

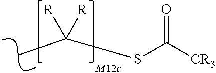

wherein M12c is 2. In another aspect of the embodiment of Formula Ib, W¹ or W² is Y^{2b}—R^{x}; each Y, Y³ and Y^{2b} is O and R^{x} is:

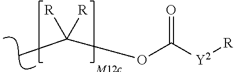

wherein M12c is 1 and Y² is a bond, O, or CR₂.

Another embodiment of

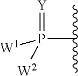

of Formula I-II compounds includes a substructure:

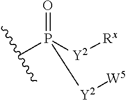

wherein W⁵ is a carbocycle such as phenyl or substituted phenyl. In another aspect of this embodiment, the substructure is:

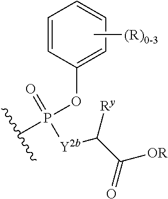

wherein Y^{2b} is O or N(R) and the phenyl carbocycle is substituted with 0 to 3 R groups. In another aspect of this embodiment of the substructure, R^{x} is:

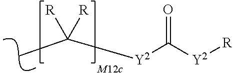

wherein M12c is 1, 2 or 3 and each Y² is independently a bond, O, CR₂, or S.

Another embodiment of

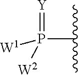

of Formula I-II includes substructures:

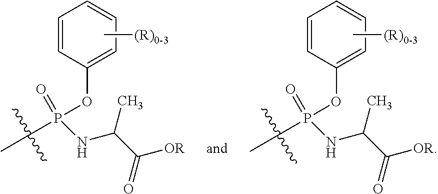

The chiral carbon of the amino acid and lactate moieties may be either the R or S configuration or the racemic mixture.

Another embodiment of

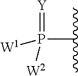

of Formula I-II is substructure

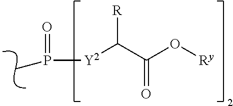

wherein each Y² is, independently, —O— or —NH—. In another aspect of this embodiment, R^{y} is (C₁-C₈) alkyl, (C₁-C₈) substituted alkyl, (C₂-C₈) alkenyl, (C₂-C₈) substituted alkenyl, (C₂-C₈) alkynyl or (C₂-C₈) substituted alkynyl. In another aspect of this embodiment. R^{y} is (C₁-C₈) alkyl, (C₁-C₈) substituted alkyl, (C₂-C₈) alkenyl, (C₂-C₈) substituted alkenyl, (C₂-C₈) alkynyl or (C₂-C₈) substituted alkynyl; and R is CH₃. In another aspect of this embodiment, R^{y} is (C₁-C₈) alkyl, (C₁-C₈) substituted alkyl, (C₂-C₈) alkenyl, (C₂-C₈) substituted alkenyl, (C₂-C₈) alkynyl or (C₂-C₈) substituted alkynyl; R is CH₃; and each Y² is —NH—. In a aspect of this embodiment, W¹ and W² are, independently, nitrogen-linked, naturally occurring amino acids or naturally occurring amino acid esters. In another aspect of this embodiment, W¹ and W² are, independently, naturally-occurring 2-hydroxy carboxylic acids or naturally-occurring 2-hydroxy carboxylic acid esters wherein the acid or ester is linked to P through the 2-hydroxy group.

Another embodiment of

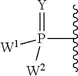

of Formula I or Formula II is substructure:

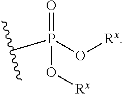

In one aspect of this embodiment, each R^{x} is, independently, (C₁-C₈) alkyl. In another aspect of this embodiment, each R¹¹ is, independently, C₆-C₂₀ aryl or C₆-C₂₀ substituted aryl.

In a preferred embodiment,

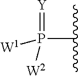

is selected from

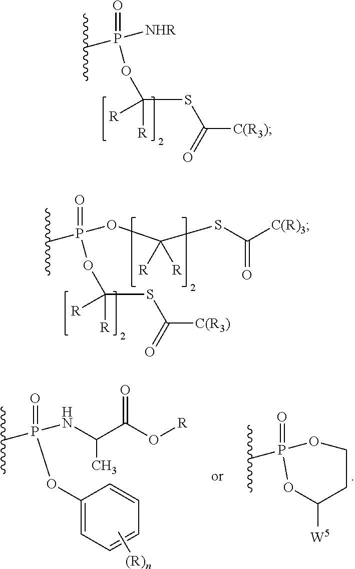

Another embodiment of

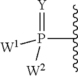

of Formulas I-II is substructure

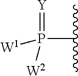

where W¹ and W² are independently selected from one of the formulas in Tables 20.1-20.37 and Table 30.1 below. The variables used in Tables 20.1-20.37 (e.g., W²³, R²¹, etc.) pertain only to Tables 20.1-20.37, unless otherwise indicated.

The variables used in Tables 20.1 to 20.37 have the following definitions:

each R²¹ is independently H or (C₁-C₈)alkyl;

each R²² is independently H, R²¹, R²³ or R²⁴ wherein each R²⁴ is independently substituted with 0 to 3 R²³;

each R²³ is independently R^{23a}, R^{23b}, R^{23c} or R^{23d}, provided that when R²³ is bound to a heteroatom, then R²³ is R^{23c} or R^{23d};

each R^{23a} is independently F, Cl, Br, I, —CN, N₃ or —NO₂;

each R^{23b} is independently Y²¹;

each R^{23c} is independently —R^{2x}, —N(R^{2x})(R^{2x}), —SR^{2x}, —S(O)R^{2x}, —S(O)₂R^{2x}, —S(OX)(OR^{2x}), —S(O)₂(OR^{2x}), —OC(═Y²¹)R^{2x}, —OC(═Y²¹)OR^{2x}, —OC(═Y²¹)(N(R^{2x})(R^{2x})), —SC(═Y²¹)R^{2x}, —SC(═Y²¹)OR^{2x}, —SC(═Y²¹)(N(R^{2x})(R^{2x})), —N(R^{2x})C(═Y²¹)R^{2x}, —N(R^{2x})C(═Y²¹)OR^{2x}, or —N(R^{2x})C(═Y²¹)(N(R^{2x})(R^{2x}));

each R^{23d} is independently —C(═Y²¹)R^{2x}, —C(═Y²¹)OR^{2x} or —C(═Y²¹)(N(R^{2x})(R^{2x}));

each R^{2x} is independently H, (C₁-C₈)alkyl, (C₂-C₈)alkenyl, (C₂-C₈)alkynyl, aryl, heteroaryl; or two R^{2x} taken together with a nitrogen to which they are both attached form a 3 to 7 membered heterocyclic ring wherein any one carbon atom of said heterocyclic ring can optionally be replaced with —O—, —S— or —NR²¹—; and wherein one or more of the non-terminal carbon atoms of each said (C₁-C₈)alkyl may be optionally replaced with —O—, —S— or —NR²¹—;

each R²⁴ is independently (C₁-C₈)alkyl, (C₂-C₈)alkenyl, or (C₂-C₈)alkynyl;

each R²⁵ is independently R²⁴ wherein each R²⁴ is substituted with 0 to 3 R²³ groups;

each R^{25a} is independently (C₁-C₈)alkylene, (C₂-C₈)alkenylene, or (C₂-C₈)alkynylene any one of which said (C₁-C₈)alkylene, (C₂-C₈)alkenylene, or (C₂-C₈)alkynylene is substituted with 0-3 R²³ groups;

each W²³ is independently W²⁴ or W²⁵;

each W²⁴ is independently R²⁵, —C(═Y²¹)R²⁵, —C(═Y²¹)W²⁵, —SO₂R²⁵, or —SO₂W²⁵;

each W²⁵ is independently carbocycle or heterocycle wherein W²⁵ is independently substituted with 0 to 3 R²² groups; and

each Y²¹ is independently O or S.

TABLE 20.1

| | |
|---|---|
| 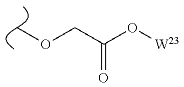 | 1 |
| 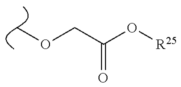 | 2 |
| 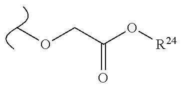 | 3 |
| 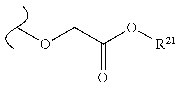 | 4 |
| 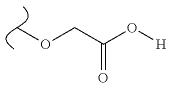 | 5 |
| 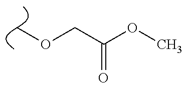 | 6 |
| 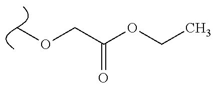 | 7 |
| 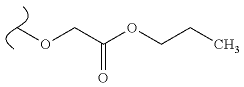 | 8 |

TABLE 20.2

| | |
|---|---|
| 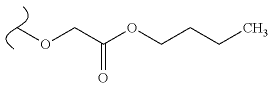 | 9 |
| 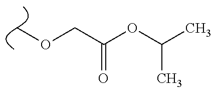 | 10 |
| 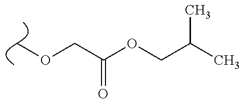 | 11 |

TABLE 20.3

| | |
|---|---|
| 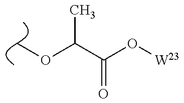 | 12 |
| 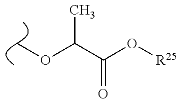 | 13 |
| 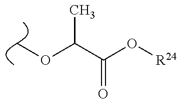 | 14 |
| 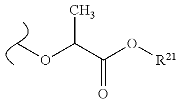 | 15 |
| 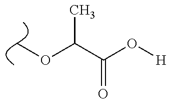 | 16 |
| 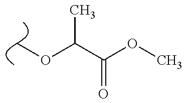 | 17 |
| 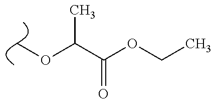 | 18 |
| 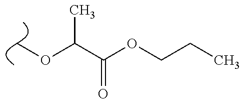 | 19 |

TABLE 20.4

| | |
|---|---|
| 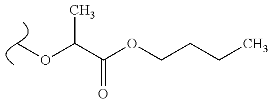 | 20 |
| 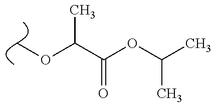 | 21 |
| 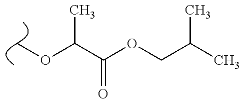 | 22 |

TABLE 20.5

| | |
|---|---|
| 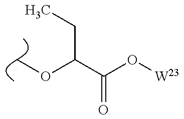 | 23 |
| 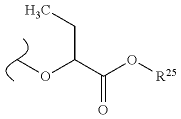 | 24 |
| 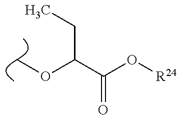 | 25 |
| 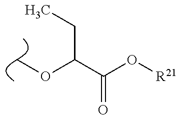 | 26 |
| 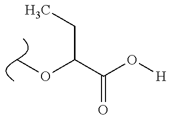 | 27 |
| 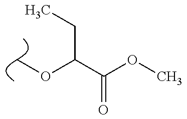 | 28 |
| 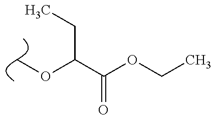 | 29 |
| 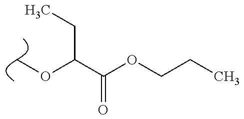 | 30 |

TABLE 20.6

| | |
|---|---|
| 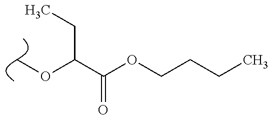 | 31 |
| 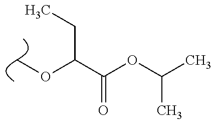 | 32 |
| 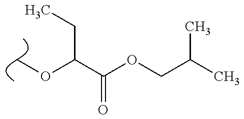 | 33 |

TABLE 20.7

| | |
|---|---|
| 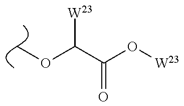 | 34 |
| 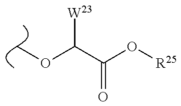 | 35 |
| 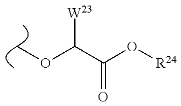 | 36 |
| 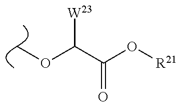 | 37 |
| 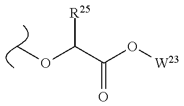 | 38 |
| 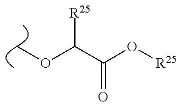 | 39 |
| 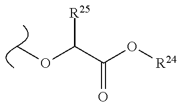 | 40 |
| 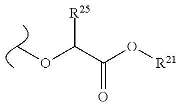 | 41 |

TABLE 20.8

| | |
|---|---|
| 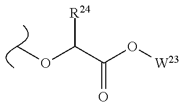 | 42 |
| 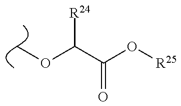 | 43 |
| 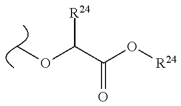 | 44 |
| 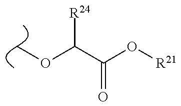 | 45 |
| 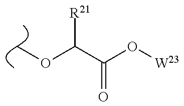 | 46 |
| 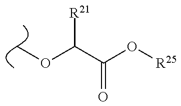 | 47 |
| 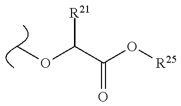 | 48 |
| 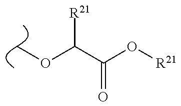 | 49 |

TABLE 20.9

| | |
|---|---|
| 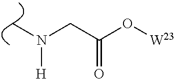 | 50 |
| 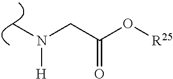 | 51 |
| 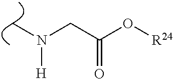 | 52 |
| 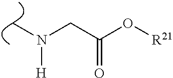 | 53 |
| 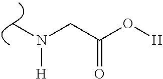 | 54 |
| 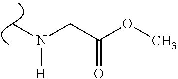 | 55 |
| 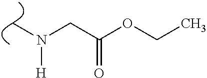 | 56 |
| 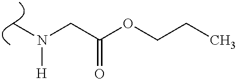 | 57 |

TABLE 20.10

| | |
|---|---|
| 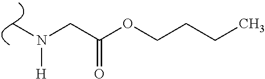 | 58 |
| 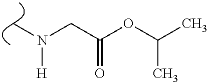 | 59 |
| 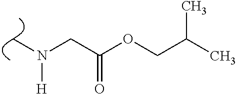 | 60 |

TABLE 20.11

| | |
|---|---|
| 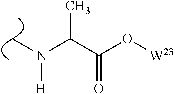 | 61 |
| 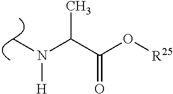 | 62 |
| 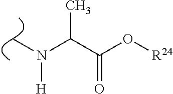 | 63 |
| 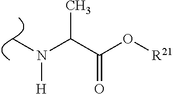 | 64 |
| 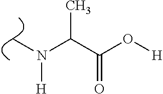 | 65 |
| 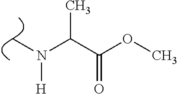 | 66 |
| 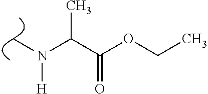 | 67 |
| 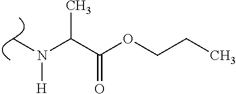 | 68 |

TABLE 20.12

| | |
|---|---|
| 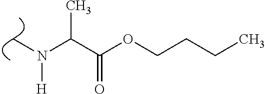 | 69 |
| 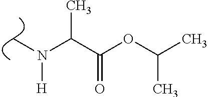 | 70 |
| 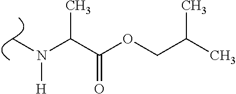 | 71 |

TABLE 20.13

| | |
|---|---|
| 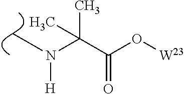 | 72 |
| 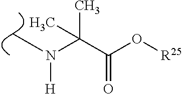 | 73 |
| 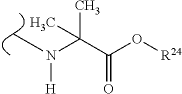 | 74 |
| 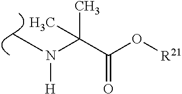 | 75 |
| 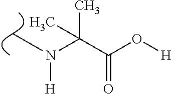 | 76 |
| 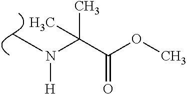 | 77 |
| 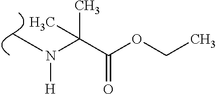 | 78 |
| 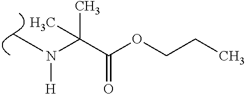 | 79 |

TABLE 20.14

| | |
|---|---|
| 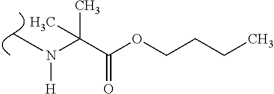 | 80 |
| 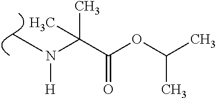 | 81 |
| 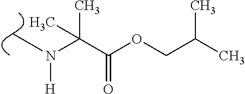 | 82 |

TABLE 20.15

| | |
|---|---|
| 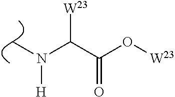 | 83 |
| 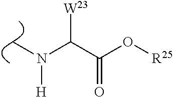 | 84 |
| 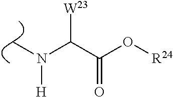 | 85 |
| 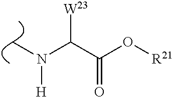 | 86 |
| 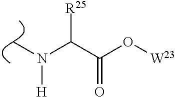 | 87 |
| 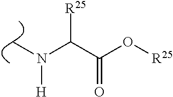 | 88 |
| 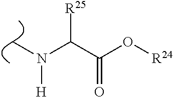 | 89 |
| 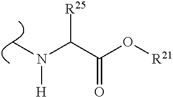 | 90 |

TABLE 20.16

| | |
|---|---|
| 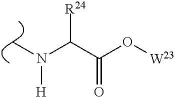 | 91 |
| 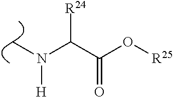 | 92 |
| 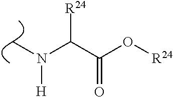 | 93 |
| 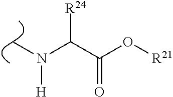 | 94 |
| 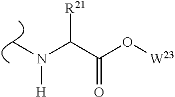 | 95 |
| 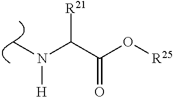 | 96 |
| 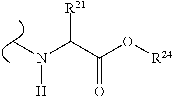 | 97 |
| 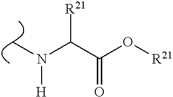 | 98 |

TABLE 20.17

| | |
|---|---|
| 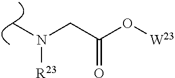 | 99 |
| 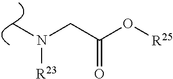 | 100 |
| 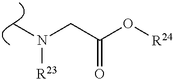 | 101 |
| 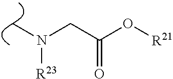 | 102 |
| 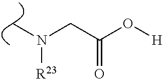 | 103 |
| 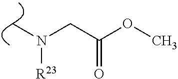 | 104 |
| 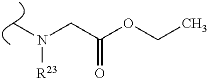 | 105 |
| 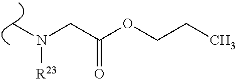 | 106 |

TABLE 20.18

| | |
|---|---|
| 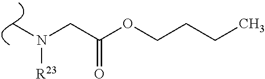 | 107 |
| 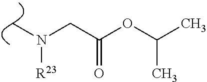 | 108 |
| 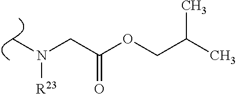 | 109 |

TABLE 20.19

| | |
|---|---|
| 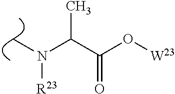 | 110 |
| 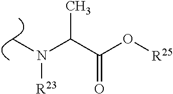 | 111 |
| 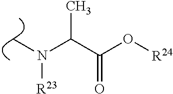 | 112 |
| 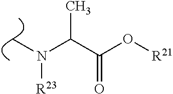 | 113 |
| 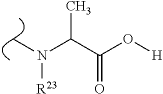 | 114 |
| 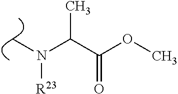 | 115 |
| 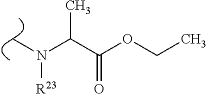 | 116 |
| 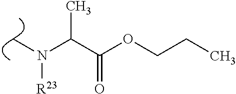 | 117 |

TABLE 20.20

| | |
|---|---|
| 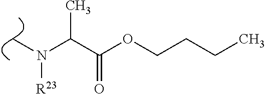 | 118 |
| 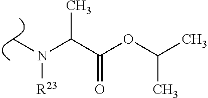 | 119 |
| 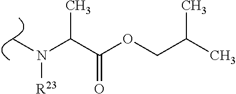 | 120 |

TABLE 20.21

| | |
|---|---|
| 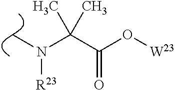 | 121 |
| 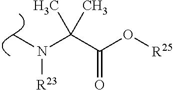 | 122 |
| 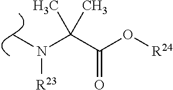 | 123 |
| 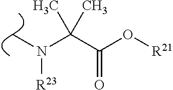 | 124 |
| 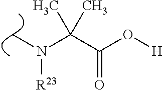 | 125 |
| 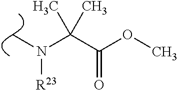 | 126 |
| 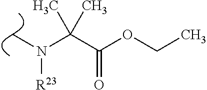 | 127 |
| 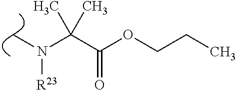 | 128 |

TABLE 20.22

| | |
|---|---|
| 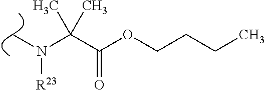 | 129 |
| 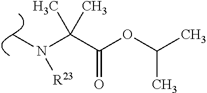 | 130 |
| 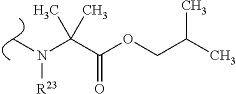 | 131 |

TABLE 20.23

| | |
|---|---|
| 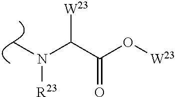 | 132 |
| 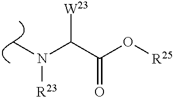 | 133 |
| 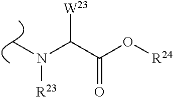 | 134 |
| 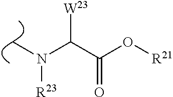 | 135 |
| 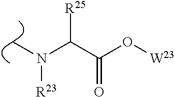 | 136 |
| 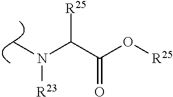 | 137 |
| 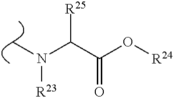 | 138 |
| 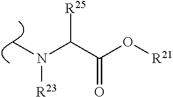 | 139 |

TABLE 20.24

| | |
|---|---|
| 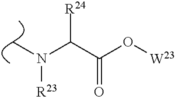 | 140 |
| 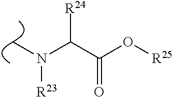 | 141 |
| 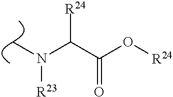 | 142 |
| 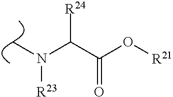 | 143 |
| 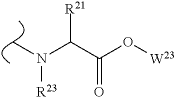 | 144 |
| 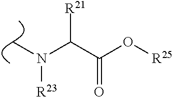 | 145 |
| 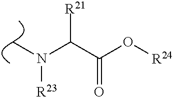 | 146 |
| 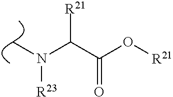 | 147 |

TABLE 20.25

| | |
|---|---|
| 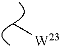 | 148 |
| 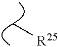 | 149 |
| 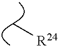 | 150 |
| 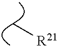 | 151 |
| 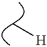 | 152 |
| 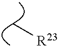 | 153 |
| 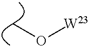 | 154 |
| 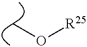 | 155 |
| 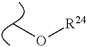 | 156 |
| 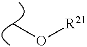 | 157 |
| 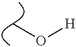 | 158 |
| 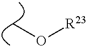 | 159 |

TABLE 20.26

| | |
|---|---|
| 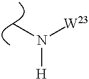 | 160 |
| 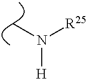 | 161 |
| 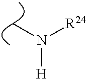 | 162 |
| 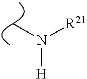 | 163 |
| 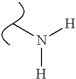 | 164 |
| 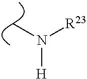 | 165 |
| 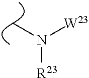 | 166 |
| 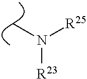 | 167 |
| 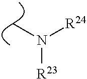 | 168 |
| 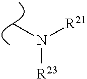 | 169 |
| 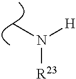 | 170 |
| 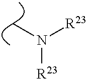 | 171 |

TABLE 20.27

| | |
|---|---|
| 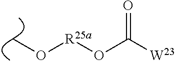 | 172 |
| 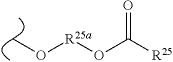 | 173 |
| 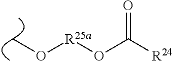 | 174 |
| 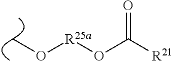 | 175 |
| 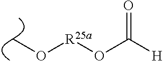 | 176 |
| 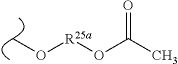 | 177 |
| 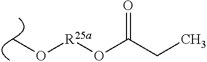 | 178 |
| 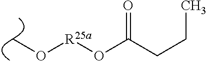 | 179 |

TABLE 20.28

| | |
|---|---|
| 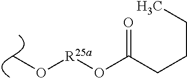 | 180 |
| 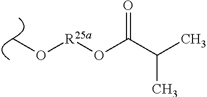 | 181 |
| 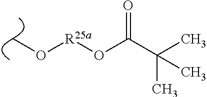 | 182 |
| 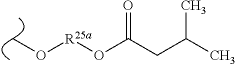 | 183 |
| 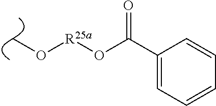 | 184 |
| 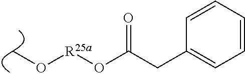 | 185 |

TABLE 20.29

| | |
|---|---|
| 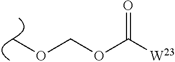 | 186 |
| 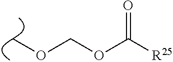 | 187 |
| 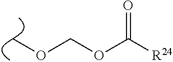 | 188 |
| 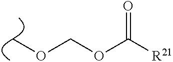 | 189 |
| 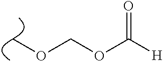 | 190 |
| 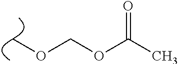 | 191 |
| 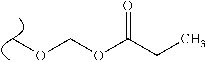 | 192 |
| 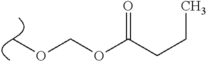 | 193 |

TABLE 20.30

| | |
|---|---|
| 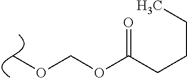 | 194 |
| 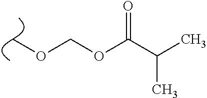 | 195 |
| 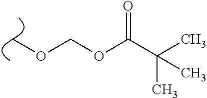 | 196 |
| 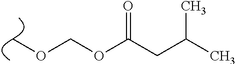 | 197 |
| 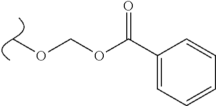 | 198 |
| 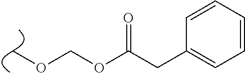 | 199 |

TABLE 20.31

| | |
|---|---|
| 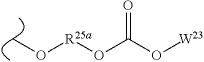 | 200 |
| 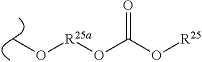 | 201 |
| 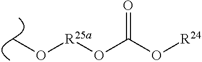 | 202 |
| 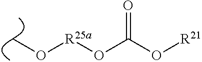 | 203 |
| 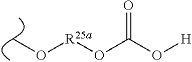 | 204 |
| 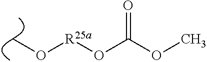 | 205 |
| 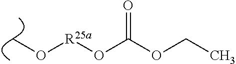 | 206 |
| 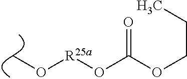 | 207 |

TABLE 20.32

| | |
|---|---|
| 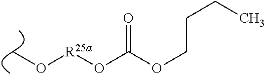 | 208 |
| 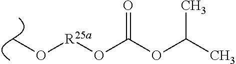 | 209 |
| 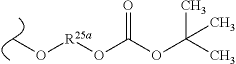 | 210 |
| 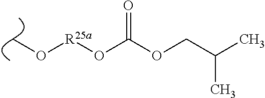 | 211 |
| 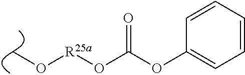 | 212 |
| 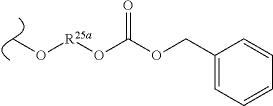 | 213 |

TABLE 20.33

| | |
|---|---|
| 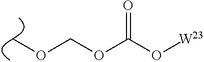 | 214 |
| 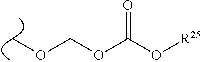 | 215 |
| 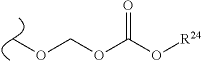 | 216 |
| 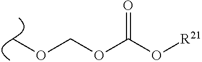 | 217 |
| 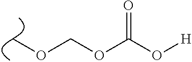 | 218 |
| 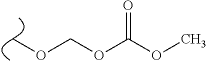 | 219 |
| 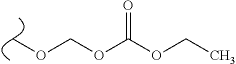 | 220 |
| 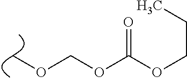 | 221 |

TABLE 20.34

| | |
|---|---|
| 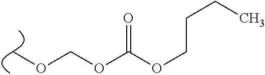 | 222 |
| 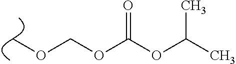 | 223 |
| 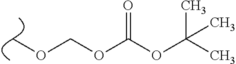 | 224 |
| 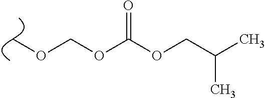 | 225 |
| 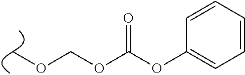 | 226 |
| 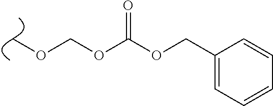 | 227 |

TABLE 20.35

| | |
|---|---|
| 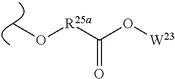 | 228 |
| 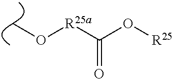 | 229 |
| 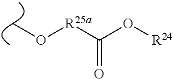 | 230 |
| 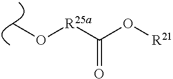 | 231 |
| 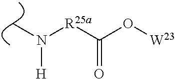 | 232 |
| 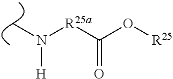 | 233 |
| 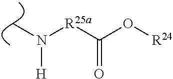 | 234 |
| 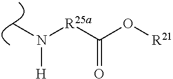 | 235 |

TABLE 20.36

| | |
|---|---|
| 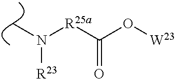 | 236 |
| 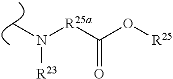 | 237 |
| 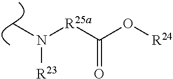 | 238 |
| 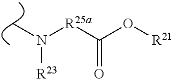 | 239 |
| 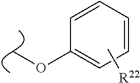 | 240 |
| 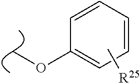 | 241 |
| 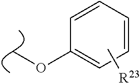 | 242 |
| 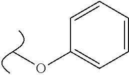 | 243 |

TABLE 20.37

| | |
|---|---|
| 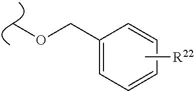 | 244 |
| 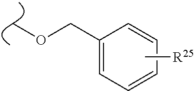 | 245 |
| 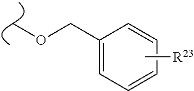 | 246 |
| 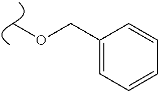 | 247 |

TABLE 30.1

| | |
|---|---|
| 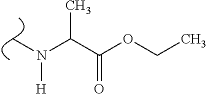 | 67 |
| 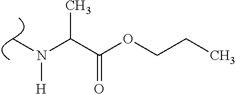 | 68 |
| 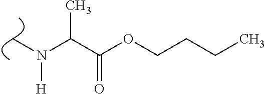 | 69 |
| 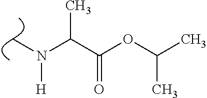 | 70 |
| 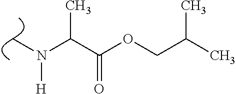 | 71 |
| 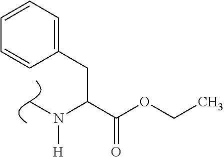 | 248 |
| 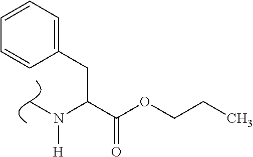 [SMILES: CCCOC(=O)C(Cc1ccccc1)NC] | 249 |
| 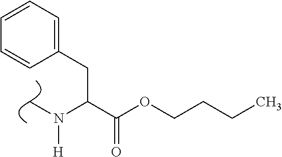 [SMILES: CCCCOC(=O)C(Cc1ccccc1)NC] | 250 |
| 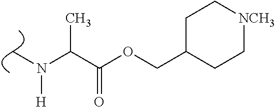 | 251 |
| 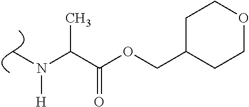 | 252 |
| 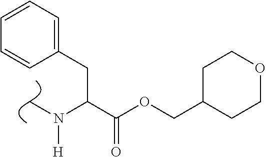 [SMILES: CNC(Cc1ccccc1)C(=O)OCC1CCOCC1] | 253 |
| 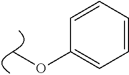 | 254 |
| 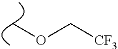 | 255 |
| 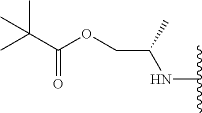 [SMILES: *N[C@@H](C)COC(=O)C(C)(C)C] | 256 |
| 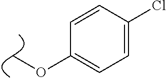 | 257 |
| 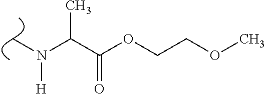 | 258 |

Embodiments of R^{x} include esters, carbamates, carbonates, thioesters, amides, thioamides, and urea groups:

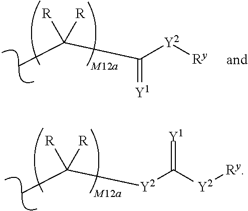

Any reference to the compounds of the invention described herein also includes a reference to a physiologically acceptable salt thereof. Examples of physiologically acceptable salts of the compounds of the invention include salts derived from an appropriate base, such as an alkali metal or an alkaline earth (for example, Na⁺, Li⁺, K⁺, Ca⁺² and Mg⁺²), ammonium and NR₄⁺ (wherein R is defined herein). Physiologically acceptable salts of a nitrogen atom or an amino group include (a) acid addition salts formed with inorganic acids, for example, hydrochloric acid, hydrobromic acid, sulfuric acid, sulfamic acids, phosphoric acid, nitric acid and the like; (b) salts formed with organic acids such as, for example, acetic acid, oxalic acid, tartaric acid, succinic acid, maleic acid, fumaric acid, gluconic acid, citric acid, malic acid, ascorbic acid, benzoic acid, isethionic acid, lactobionic acid, tannic acid, palmitic acid, alginic acid, polyglutamic acid, naphthalenesulfonic acid, methanesulfonic acid, p-toluenesulfonic acid, benzenesulfonic acid, naphthalenedisulfonic acid, polygalacturonic acid, malonic acid, sulfosalicylic acid, glycolic acid, 2-hydroxy-3-naphthoate, pamoate, salicylic acid, stearic acid, phthalic acid, mandelic acid, lactic acid, ethanesulfonic acid, lysine, arginine, glutamic acid, glycine, serine, threonine, alanine, isoleucine, leucine and the like; and (c) salts formed from elemental anions for example, chlorine, bromine, and iodine. Physiologically acceptable salts of a compound of a hydroxy group include the anion of said compound in combination with a suitable cation such as Na⁺ and NR₄⁺.

For therapeutic use, salts of active ingredients of the compounds of the invention will be physiologically acceptable, i.e. they will be salts derived from a physiologically acceptable acid or base. However, salts of acids or bases which are not physiologically acceptable may also find use, for example, in the preparation or purification of a physiologically acceptable compound. All salts, whether or not derived form a physiologically acceptable acid or base, are within the scope of the present invention.

Finally, it is to be understood that the compositions herein comprise compounds of the invention in their un-ionized, as well as zwitterionic form, and combinations with stoichiometric amounts of water as in hydrates.

The compounds of the invention, exemplified by Formula I-II may have chiral centers, e.g. chiral carbon or phosphorus atoms. The compounds of the invention thus include racemic mixtures of all stereoisomers, including enantiomers, diastereomers, and atropisomers. In addition, the compounds of the invention include enriched or resolved optical isomers at any or all asymmetric, chiral atoms. In other words, the chiral centers apparent from the depictions are provided as the chiral isomers or racemic mixtures. Both racemic and diastereomeric mixtures, as well as the individual optical isomers isolated or synthesized, substantially free of their enantiomeric or diastereomeric partners, are all within the scope of the invention. The racemic mixtures are separated into their individual, substantially optically pure isomers through well-known techniques such as, for example, the separation of diastereomeric salts formed with optically active adjuncts, e.g., acids or bases followed by conversion back to the optically active substances. In most instances, the desired optical isomer is synthesized by means of stereospecific reactions, beginning with the appropriate stereoisomer of the desired starting material.

The term “chiral” refers to molecules which have the property of non-superimposability of the mirror image partner, while the term “achiral” refers to molecules which are superimposable on their mirror image partner.

The term “stereoisomers” refers to compounds which have identical chemical constitution, but differ with regard to the arrangement of the atoms or groups in space.

“Diastereomer” refers to a stereoisomer with two or more centers of chirality and whose molecules are not mirror images of one another. Diastereomers have different physical properties, e.g. melting points, boiling points, spectral properties, and reactivities. Mixtures of diastereomers may separate under high resolution analytical procedures such as electrophoresis and chromatography.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel. E. and Wilen, S., Stereochemistry of Organic Compounds (1994) John Wiley & Sons, Inc., New York. Many organic compounds exist in optically active forms, i.e., they have the ability to rotate the plane of plane-polarized light. In describing an optically active compound, the prefixes D and L or R and S are used to denote the absolute configuration of the molecule about its chiral center(s). The prefixes d and l, D and L, or (+) and (−) are employed to designate the sign of rotation of plane-polarized light by the compound, with S, (−), or l meaning that the compound is levorotatory while a compound prefixed with R, (+), or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

Whenever a compound described herein is substituted with more than one of the same designated group, e.g., “R” or “R₁”, then it will be understood that the groups may be the same or different, i.e., each group is independently selected. Wavy lines, [symbol] , indicate the site of covalent bond attachments to the adjoining substructures, groups, moieties, or atoms.

The compounds of the invention can also exist as tautomeric isomers in certain cases. Although only one delocalized resonance structure may be depicted, all such forms are contemplated within the scope of the invention. For example, ene-amine tautomers can exist for purine, pyrimidine, imidazole, guanidine, amidine, and tetrazole systems and all their possible tautomeric forms are within the scope of the invention.

### Methods of Inhibition of Orthomyxoviridae RNA-Dependent RNA Polymerase

Another aspect of the invention relates to methods of inhibiting the activity of Orthomyxoviridae polymerase comprising the step of treating a sample suspected of containing Orthomyxoviridae virus with a composition of the invention.

Compositions of the invention may act as inhibitors of Orthomyxoviridae polymerase, as intermediates for such inhibitors or have other utilities as described below. The inhibitors will bind to locations on the surface or in a cavity of Orthomyxoviridae polymerase having a geometry unique to Orthomyxoviridae polymerase. Compositions binding Orthomyxoviridae polymerase may bind with varying degrees of reversibility. Those compounds binding substantially irreversibly are ideal candidates for use in this method of the invention. Once labeled, the substantially irreversibly binding compositions are useful as probes for the detection of Orthomyxoviridae polymerase. Accordingly, the invention relates to methods of detecting Orthomyxoviridae polymerase in a sample suspected of containing Orthomyxoviridae polymerase comprising the steps of: treating a sample suspected of containing Orthomyxoviridae polymerase with a composition comprising a compound of the invention bound to a label; and observing the effect of the sample on the activity of the label. Suitable labels are well known in the diagnostics field and include stable free radicals, fluorophores, radioisotopes, enzymes, chemiluminescent groups and chromogens. The compounds herein are labeled in conventional fashion using functional groups such as hydroxyl, carboxyl, sulfhydryl or amino.

Within the context of the invention, samples suspected of containing Orthomyxoviridae polymerase include natural or man-made materials such as living organisms; tissue or cell cultures; biological samples such as biological material samples (blood, serum, urine, cerebrospinal fluid, tears, sputum, saliva, tissue samples, and the like); laboratory samples; food, water, or air samples; bioproduct samples such as extracts of cells, particularly recombinant cells synthesizing a desired glycoprotein; and the like. Typically the sample will be suspected of containing an organism which produces Orthomyxoviridae polymerase, frequently a pathogenic organism such as Orthomyxoviridae virus. Samples can be contained in any medium including water and organic solvent\water mixtures. Samples include living organisms such as humans, and man made materials such as cell cultures.

The treating step of the invention comprises adding the composition of the invention to the sample or it comprises adding a precursor of the composition to the sample. The addition step comprises any method of administration as described herein.

If desired, the activity of Orthomyxoviridae polymerase after application of the composition can be observed by any method including direct and indirect methods of detecting Orthomyxoviridae polymerase activity. Quantitative, qualitative, and semiquantitative methods of determining Orthomyxoviridae polymerase activity are all contemplated. Typically one of the screening methods described above are applied, however, any other method such as observation of the physiological properties of a living organism are also applicable.

Organisms that contain Orthomyxoviridae polymerase include the Orthomyxoviridae virus. The compounds of this invention are useful in the treatment or prophylaxis of Orthomyxoviridae infections in animals or in man.

In still yet another embodiment, the present application provides for methods of inhibiting Orthomyxoviridae RNA-dependent RNA polymerase in a cell, comprising: contacting a cell infected with Orthomyxoviridae virus with an effective amount of a compound of Formula I-II, or a pharmaceutically acceptable salt, solvate, and/or ester thereof, whereby the Orthomyxoviridae polymerase is inhibited.

In still yet another embodiment, the present application provides for methods of inhibiting Orthomyxoviridae polymerase in a cell, comprising: contacting a cell infected with Orthomyxoviridae virus with an effective amount of a compound of Formula I-II, or a pharmaceutically acceptable salt, solvate, and/or ester thereof, and at least one additional active therapeutic agent, whereby the Orthomyxoviridae polymerase is inhibited.

In still yet another embodiment, the present application provides for methods of inhibiting Orthomyxoviridae polymerase in a cell, comprising: contacting a cell infected with Orthomyxoviridae virus with an effective amount of a compound of Formula I-II, or a pharmaceutically acceptable salt, solvate, and/or ester thereof, and at least one additional active therapeutic agent selected from the group consisting of interferons, ribavirin analogs, viral neuramidase inhibitors, viral neuramidase inhibitors. M2 ion channel blockers. Orthomyxoviridae RNA-dependent RNA polymerases inhibitors, sialidases and other drugs used to treat Orthomyxoviridae virus infections.

### Pharmaceutical Formulations

The compounds of this invention are formulated with conventional carriers and excipients, which will be selected in accord with ordinary practice. Tablets will contain excipients, glidants, fillers, binders and the like. Aqueous formulations are prepared in sterile form, and when intended for delivery by other than oral administration generally will be isotonic. All formulations will optionally contain excipients such as those set forth in the “Handbook of Pharmaceutical Excipients” (1986). Excipients include ascorbic acid and other antioxidants, chelating agents such as EDTA, carbohydrates such as dextran, hydroxyalkylcellulose, hydroxyalkylmethylcellulose, stearic acid and the like. The pH of the formulations ranges from about 3 to about 11, but is ordinarily about 7 to 10.

While it is possible for the active ingredients to be administered alone it may be preferable to present them as pharmaceutical formulations. The formulations, both for veterinary and for human use, of the invention comprise at least one active ingredient, as above defined, together with one or more acceptable carriers therefor and optionally other therapeutic ingredients. The carrier(s) must be “acceptable” in the sense of being compatible with the other ingredients of the formulation and physiologically innocuous to the recipient thereof.

The formulations include those suitable for the foregoing administration routes. The formulations may conveniently be presented in unit dosage form and may be prepared by any of the methods well known in the art of pharmacy. Techniques and formulations generally are found in Remington's Pharmaceutical Sciences (Mack Publishing Co., Easton, Pa.). Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations of the present invention suitable for oral administration may be presented as discrete units such as capsules, cachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be administered as a bolus, electuary or paste.

A tablet is made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom.

For infections of the eye or other external tissues e.g. mouth and skin, the formulations are preferably applied as a topical ointment or cream containing the active ingredient(s) in an amount of, for example, 0.075 to 20% w/w (including active ingredient(s) in a range between 0.1% and 20% in increments of 0.1% w/w such as 0.6% w/w, 0.7% w/w, etc.), preferably 0.2 to 15% w/w and most preferably 0.5 to 10% w/w. When formulated in an ointment, the active ingredients may be employed with either a paraffinic or a water-miscible ointment base. Alternatively, the active ingredients may be formulated in a cream with an oil-in-water cream base.

If desired, the aqueous phase of the cream base may include, for example, at least 30% w/w of a polyhydric alcohol. i.e. an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound which enhances absorption or penetration of the active ingredient through the skin or other affected areas. Examples of such dermal penetration enhancers include dimethyl sulphoxide and related analogs.

The oily phase of the emulsions of this invention may be constituted from known ingredients in a known manner. While the phase may comprise merely an emulsifier (otherwise known as an emulgent), it desirably comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations.

Emulgents and emulsion stabilizers suitable for use in the formulation of the invention include Tween® 60, Span® 80, cetostearyl alcohol, benzyl alcohol, myristyl alcohol, glyceryl mono-stearate and sodium lauryl sulfate.

The choice of suitable oils or fats for the formulation is based on achieving the desired cosmetic properties. The cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils are used.

Pharmaceutical formulations according to the present invention comprise a combination according to the invention together with one or more pharmaceutically acceptable carriers or excipients and optionally other therapeutic agents. Pharmaceutical formulations containing the active ingredient may be in any form suitable for the intended method of administration. When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient is mixed with an inert solid diluent for example calcium phosphate or kaolin, or as soft gelatin capsules wherein the active ingredient is mixed with water or an oil medium, such as peanut oil, liquid paraffin or olive oil.

Aqueous suspensions of the invention contain the active materials in admixture with excipients suitable for the manufacture of aqueous suspensions. Such excipients include a suspending agent, such as sodium carboxymethylcellulose, methylcellulose, hydroxypropyl methylcelluose, sodium alginate, polyvinylpyrrolidone, gum tragacanth and gum acacia, and dispersing or wetting agents such as a naturally-occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin.

Oil suspensions may be formulated by suspending the active ingredient in a vegetable oil, such as arachis oil, olive oil, sesame oil or coconut oil, or in a mineral oil such as liquid paraffin. The oral suspensions may contain a thickening agent, such as beeswax, hard paraffin or cetyl alcohol. Sweetening agents, such as those set forth above, and flavoring agents may be added to provide a palatable oral preparation. These compositions may be preserved by the addition of an antioxidant such as ascorbic acid.

Dispersible powders and granules of the invention suitable for preparation of an aqueous suspension by the addition of water provide the active ingredient in admixture with a dispersing or wetting agent, a suspending agent, and one or more preservatives. Suitable dispersing or wetting agents and suspending agents are exemplified by those disclosed above. Additional excipients, for example sweetening, flavoring and coloring agents, may also be present.

The pharmaceutical compositions of the invention may also be in the form of oil-in-water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth, naturally-occurring phosphatides, such as soybean lecithin, esters or partial esters derived from fatty acids and hexitol anhydrides, such as sorbitan monooleate, and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring or a coloring agent.

The pharmaceutical compositions of the invention may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

The amount of active ingredient that may be combined with the carrier material to produce a single dosage form will vary depending upon the host treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur.

Formulations suitable for topical administration to the eye also include eye drops wherein the active ingredient is dissolved or suspended in a suitable carrier, especially an aqueous solvent for the active ingredient. The active ingredient is preferably present in such formulations in a concentration of 0.5 to 20%, advantageously 0.5 to 10%, and particularly about 1.5% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

Formulations suitable for intrapulmonary or nasal administration have a particle size for example in the range of 0.1 to 500 microns, such as 0.5, 1, 30, 35 etc., which is administered by rapid inhalation through the nasal passage or by inhalation through the mouth so as to reach the alveolar sacs. Suitable formulations include aqueous or oily solutions of the active ingredient. Formulations suitable for aerosol or dry powder administration may be prepared according to conventional methods and may be delivered with other therapeutic agents such as compounds heretofore used in the treatment or prophylaxis of Orthomyxoviridae infections as described below.

In another aspect, the invention is a novel, efficacious, safe, nonirritating and physiologically compatible inhalable composition comprising a compound of Formula I-II, or a pharmaceutically acceptable salt thereof, suitable for treating Orthomyxoviridae infections and potentially associated bronchiolitis. Preferred pharmaceutically acceptable salts are inorganic acid salts including hydrochloride, hydrobromide, sulfate or phosphate salts as they may cause less pulmonary irritation. Preferably, the inhalable formulation is delivered to the endobronchial space in an aerosol comprising particles with a mass median aerodynamic diameter (MMAD) between about 1 and about 5 μm. Preferably, the compound of Formula I-II is formulated for aerosol delivery using a nebulizer, pressurized metered dose inhaler (pMDI), or dry powder inhaler (DPI).

Non-limiting examples of nebulizers include atomizing, jet, ultrasonic, pressurized, vibrating porous plate, or equivalent nebulizers including those nebulizers utilizing adaptive aerosol delivery technology (Denyer J. Aerosol medicine Pulmonary Drug Delivery 2010, 23 Supp 1, S1-S10). A jet nebulizer utilizes air pressure to break a liquid solution into aerosol droplets. An ultrasonic nebulizer works by a piezoelectric crystal that shears a liquid into small aerosol droplets. A pressurized nebulization system forces solution under pressure through small pores to generate aerosol droplets. A vibrating porous plate device utilizes rapid vibration to shear a stream of liquid into appropriate droplet sizes.

In a preferred embodiment, the formulation for nebulization is delivered to the endobronchial space in an aerosol comprising particles with a MMAD predominantly between about 1 μm and about 5 μm using a nebulizer able to aerosolize the formulation of the compound of Formula I-II into particles of the required MMAD. To be optimally therapeutically effective and to avoid upper respiratory and systemic side effects, the majority of aerosolized particles should not have a MMAD greater than about 5 μm. It an aerosol contains a large number of particles with a MMAD larger than 5 μm, the particles are deposited in the upper airways decreasing the amount of drug delivered to the site of inflammation and bronchoconstriction in the lower respiratory tract. If the MMAD of the aerosol is smaller than about 1 μm, then the particles have a tendency to remain suspended in the inhaled air and are subsequently exhaled during expiration.

When formulated and delivered according to the method of the invention, the aerosol formulation for nebulization delivers a therapeutically efficacious dose of the compound of Formula I-II to the site of Orthomyxoviridae infection sufficient to treat the Orthomyxoviridae infection. The amount of drug administered must be adjusted to reflect the efficiency of the delivery of a therapeutically efficacious dose of the compound of Formula I-II. In a preferred embodiment, a combination of the aqueous aerosol formulation with the atomizing, jet, pressurized, vibrating porous plate, or ultrasonic nebulizer permits, depending on the nebulizer, about, at least, 20, to about 90%, typically about 70% delivery of the administered dose of the compound of Formula I-II into the airways. In a preferred embodiment, at least about 30 to about 50%, of the active compound is delivered. More preferably, about 70 to about 90% of the active compound is delivered.

In another embodiment of the instant invention, a compound of Formula I-II or a pharmaceutically acceptable salt thereof, is delivered as a dry inhalable powder. The compounds of the invention are administered endobronchially as a dry powder formulation to efficacious deliver fine particles of compound into the endobronchial space using dry powder or metered dose inhalers. For delivery by DPI, the compound of Formula I-II is processed into particles with, predominantly, MMAD between about 1 μm and about 5 μm by milling spray drying, critical fluid processing, or precipitation from solution. Media milling, jet milling and spray-drying devices and procedures capable of producing the particle sizes with a MMAD between about 1 μm and about 5 μm are well know in the art. In one embodiment, excipients are added to the compound of Formula I-II before processing into particles of the required sizes. In another embodiment, excipients are blended with the particles of the required size to aid in dispersion of the drug particles, for example by using lactose as an excipient.

Particle size determinations are made using devices well known in the art. For example a multi-stage Anderson cascade impactor or other suitable method such as those specifically cited within the US Pharmacopoeia Chapter 601 as characterizing devices for aerosols within metered-dose and dry powder inhalers.

In another preferred embodiment, a compound of Formula I-II is delivered as a dry powder using a device such as a dry powder inhaler or other dry powder dispersion devices. Non-limiting examples of dry powder inhalers and devices include those disclosed in U.S. Pat. No. 5,458,135; U.S. Pat. No. 5,740,794; U.S. Pat. No. 5,775,320; U.S. Pat. No. 5,785,049; U.S. Pat. No. 3,906,950; U.S. Pat. No. 4,013,075; U.S. Pat. No. 4,069,819; U.S. Pat. No. 4,995,385; U.S. Pat. No. 5,522,385; U.S. Pat. No. 4,668,218; U.S. Pat. No. 4,667,668; U.S. Pat. No. 4,805,811 and U.S. Pat. No. 5,388,572. There are two major designs of dry powder inhalers. One design is a metering device in which a reservoir for the drug is place within the device and the patient adds a dose of the drug into the inhalation chamber. The second design is a factory-metered device in which each individual dose has been manufactured in a separate container. Both systems depend on the formulation of the drug into small particles of MMAD from 1 μm and about 5 μm, and often involve co-formulation with larger excipient particles such as, but not limited to, lactose. Drug powder is placed in the inhalation chamber (either by device metering or by breakage of a factory-metered dosage) and the inspiratory flow of the patient accelerates the powder out of the device and into the oral cavity. Non-laminar flow characteristics of the powder path cause the excipient-drug aggregates to decompose, and the mass of the large excipient particles causes their impaction at the back of the throat, while the smaller drug particles are deposited deep in the lungs. In preferred embodiments, a compound of Formula I-II, or a pharmaceutically acceptable salt thereof, is delivered as a dry powder using either type of dry powder inhaler as described herein, wherein the MMAD of the dry powder, exclusive of any excipients, is predominantly in the range of 1 μm to about 5 μm.

In another preferred embodiment, a compound of Formula I-II is delivered as a dry powder using a metered dose inhaler. Non-limiting examples of metered dose inhalers and devices include those disclosed in U.S. Pat. No. 5,261,538; U.S. Pat. No. 5,544,647; U.S. Pat. No. 5,622,163; U.S. Pat. No. 4,955,371; U.S. Pat. No. 3,565,070; U.S. Pat. No. 3,361,306 and U.S. Pat. No. 6,116,234. In preferred embodiments, a compound of Formula I-II, or a pharmaceutically acceptable salt thereof, is delivered as a dry powder using a metered dose inhaler wherein the MMAD of the dry powder, exclusive of any excipients, is predominantly in the range of about 1-5 μm.

Formulations suitable for vaginal administration may be presented as pessaries, tampons, creams, gels, pastes, foams or spray formulations containing in addition to the active ingredient such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations are presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

It should be understood that in addition to the ingredients particularly mentioned above the formulations of this invention may include other agents conventional in the art having regard to the type of formulation in question, for example those suitable for oral administration may include flavoring agents.

The invention further provides veterinary compositions comprising at least one active ingredient as above defined together with a veterinary carrier therefor.

Veterinary carriers are materials useful for the purpose of administering the composition and may be solid, liquid or gaseous materials which are otherwise inert or acceptable in the veterinary art and are compatible with the active ingredient. These veterinary compositions may be administered orally, parenterally or by any other desired route.

Compounds of the invention are used to provide controlled release pharmaceutical formulations containing as active ingredient one or more compounds of the invention (“controlled release formulations”) in which the release of the active ingredient are controlled and regulated to allow less frequency dosing or to improve the pharmacokinetic or toxicity profile of a given active ingredient.

### Effective Dosage

The effective dose of active ingredient depends, at least, on the nature of the condition being treated, toxicity, whether the compound is being used prophylactically (lower doses) or against an active viral infection, the method of delivery, and the pharmaceutical formulation, and will be determined by the clinician using conventional dose escalation studies. It can be expected to be from about 0.0001 to about 100 mg/kg body weight per day; typically, from about 0.01 to about 10 mg/kg body weight per day; more typically, from about 0.01 to about 5 mg/kg body weight per day; most typically, from about 0.05 to about 0.5 mg/kg body weight per day. For example, the daily candidate dose for an adult human of approximately 70 kg body weight will range from 1 mg to 1000 mg, preferably between 5 mg and 500 mg, and may take the form of single or multiple doses.

### Routes of Administration

One or more compounds of the invention (herein referred to as the active ingredients) are administered by any route appropriate to the condition to be treated. Suitable routes include oral, inhalation, rectal, nasal, topical (including buccal and sublingual), vaginal and parenteral (including subcutaneous, intramuscular, intravenous, intradermal, intrathecal and epidural), and the like. It will be appreciated that the preferred route may vary with for example the condition of the recipient.

### Combination Therapy

In another embodiment, the present application discloses pharmaceutical compositions comprising a compound of the present invention, or a pharmaceutically acceptable salt, solvate, and/or ester thereof, in combination with at least one additional therapeutic agent, and a pharmaceutically acceptable carrier or excipient.

For the treatment of Orthomyxoviridae virus infections, preferably, the other active therapeutic agent is active against Orthomyxoviridae virus infections, particularly Influenza virus infections. Non-limiting examples of these other active therapeutic agents are viral haemagglutinin inhibitors, viral neuramidase inhibitors, M2 ion channel blockers, Orthomyxoviridae RNA-dependent RNA polymerases inhibitors and sialidases. Non-limiting examples of neuramidase inhibitors include oseltamivir, zanamivir, laninamivir, peramivir and CS-8958. Non-limiting examples of viral M2 channel inhibitors include amantadine and rimantadine. Non-limiting examples of Orthomyxoviridae RNA-dependent RNA polymerases inhibitors are ribavirin and favipiravir. Non-limiting examples of sialidases are DAS181.

Many of the infections of the Orthomyxoviridae viruses are respiratory infections. Therefore, additional active therapeutics used to treat respiratory symptoms and sequalae of infection may be used in combination with the compounds of Formula I-II. For example, other preferred additional therapeutic agents in combination with the compounds of Formula I-II for the treatment of viral respiratory infections include, but are not limited to, bronchodilators and corticosteroids.

Glucocorticoids, which were first introduced as an asthma therapy in 1950 (Carryer, Journal of Allergy, 21, 282-287, 1950), remain the most potent and consistently effective therapy for this disease, although their mechanism of action is not yet fully understood (Morris, J. Allergy Clin. Immunol., 75 (1 Pt) 1-13, 1985). Unfortunately, oral glucocorticoid therapies are associated with profound undesirable side effects such as truncal obesity, hypertension, glaucoma, glucose intolerance, acceleration of cataract formation, bone mineral loss, and psychological effects, all of which limit their use as long-term therapeutic agents (Goodman and Gilman, 10th edition, 2001). A solution to systemic side effects is to deliver steroid drugs directly to the site of inflammation. Inhaled corticosteroids (ICS) have been developed to mitigate the severe adverse effects of oral steroids. Non-limiting examples of corticosteroids that may be used in combinations with the compounds of Formula I-II are dexamethasone, dexamethasone sodium phosphate, fluorometholone, fluorometholone acetate, loteprednol, loteprednol etabonate, hydrocortisone, prednisolone, fludrocortisones, triamcinolone, triamcinolone acetonide, betamethasone, beclomethasone diproprionate, methylprednisolone, fluocinolone, fluocinolone acetonide, flunisolide, fluocortin-21-butylate, flumethasone, flumetasone pivalate, budesonide, halobetasol propionate, mometasone furoate, fluticasone propionate, ciclesonide; or a pharmaceutically acceptable salts thereof.

Other anti-inflamatory agents working through anti-inflamatory cascade mechanisms are also useful as additional therapeutic agents in combination with the compounds of Formula I-II for the treatment of viral respiratory infections. Applying “anti-inflammatory signal transduction modulators” (referred to in this text as AISTM), like phosphodiesterase inhibitors (e.g. PDE-4, PDE-5, or PDE-7 specific), transcription factor inhibitors (e.g. blocking NFκB through IKK inhibition), or kinase inhibitors (e.g. blocking P38 MAP, JNK, PI3K, EGFR or Syk) is a logical approach to switching off inflammation as these small molecules target a limited number of common intracellular pathways—those signal transduction pathways that are critical points for the anti-inflammatory therapeutic intervention (see review by P. J. Barnes, 2006). These non-limiting additional therapeutic agents include: 5-(2,4-Difluoro-phenoxy)-1-isobutyl-1H-indazole-6-carboxylic acid (2-dimethylamino-ethyl)-amide (P38 Map kinase inhibitor ARRY-797); 3-Cyclopropylmethoxy-N-(3,5-dichloro-pyridin-4-yl)-4-difluoromethoxy-benzamide (PDE-4 inhibitor Roflumilast); 4-[2-(3-cyclopentyloxy-4-methoxyphenyl)-2-phenyl-ethyl]-pyridine (PDE-4 inhibitor CDP-840); N-(3,5-dichloro-4-pyridinyl)-4-(difluoromethoxy)-8-[(methylsulfonyl)amino]-1-dibenzofurancarboxamide (PDE-4 inhibitor Oglemilast); N-(3,5-Dichloro-pyridin-4-yl)-2-[1-(4-fluorobenzyl)-5-hydroxy-1H-indol-3-yl]-2-oxo-acetamide (PDE-4 inhibitor AWD 12-281); 8-Methoxy-2-trifluoromethyl-quinoline-5-carboxylic acid (3,5-dichloro-1-oxy-pyridin-4-yl)-amide (PDE-4 inhibitor Sch 351591); 4-[5-(4-Fluorophenyl)-2-(4-methanesulfinyl-phenyl)-1H-imidazol-4-yl]-pyridine (P38 inhibitor SB-203850); 4-[4-(4-Fluoro-phenyl)-1-(3-phenyl-propyl)-5-pyridin-4-yl-1H-imidazol-2-yl]-but-3-yn-1-ol (P38 inhibitor RWJ-67657); 4-Cyano-4-(3-cyclopentyloxy-4-methoxy-phenyl)-cyclohexanecarboxylic acid 2-diethylamino-ethyl ester (2-diethyl-ethyl ester prodrug of Cilomilast, PDE-4 inhibitor); (3-Chloro-4-fluorophenyl)-[7-methoxy-6-(3-morpholin-4-yl-propoxy)-quinazolin-4-yl]-amine (Gefitinib, EGFR inhibitor); and 4-(4-Methyl-piperazin-1-ylmethyl)-N-[4-methyl-3-(4-pyridin-3-yl-pyrimidin-2-ylamino)-phenyl]-benzamide (Imatinib, EGFR inhibitor).

Combinations comprising inhaled β2-adrenoreceptor agonist bronchodilators such as formoterol, albuterol or salmeterol with the compounds of Formula I-II are also suitable, but non-limiting, combinations useful for the treatment of respiratory viral infections.

Combinations of inhaled β2-adrenoreceptor agonist bronchodilators such as formoterol or salmeterol with ICS's are also used to treat both the bronchoconstriction and the inflammation (Symbicort®, and Advair®, respectively). The combinations comprising these ICS and β2-adrenoreceptor agonist combinations along with the compounds of Formula I-II are also suitable, but non-limiting, combinations useful for the treatment of respiratory viral infections.

For the treatment or prophylaxis of pulmonary broncho-constriction, anticholinergics are of potential use and, therefore, useful as an additional therapeutic agents in combination with the compounds of Formula I-II for the treatment of viral respiratory infections. These anticholinergics include, but are not limited to, antagonists of the muscarinic receptor (particularly of the M3 subtype) which have shown therapeutic efficacy in man for the control of cholinergic tone in COPD (Witek, 1999); 1-{4-Hydroxy-1-[3,3,3-tris-(4-fluoro-phenyl)-propionyl]-pyrrolidine-2-carbonyl}-pyrrolidine-2-carboxylic acid (1-methyl-piperidin-4-ylmethyl)-amide; 3-[3-(2-Dithylamino-acetoxy)-2-phenyl-propionyloxy]-8-isopropyl-8-methyl-8-azonia-bicyclo[3.2.1]octane (Ipratropium-N,N-diethylglycinate); 1-Cyclohexyl-3,4-dihydro-1 H-isoquinoline-2-carboxylic acid 1-aza-bicyclo[2.2.2]oct-3-yl ester (Solifenacin); 2-Hydroxymethyl-4-methanesulfinyl-2-phenyl-butyric acid 1-aza-bicyclo[2.2.2]oct-3-yl ester (Revatropate); 2-{1-[2-(2,3-Dihydro-benzofuran-5-yl)-ethyl]-pyrrolidin-3-yl}-2,2-diphenyl-acetamide (Darifenacin); 4-Azepan-1-yl-2,2-diphenyl-butyramide (Buzepide); 7-[3-(2-Diethylamino-acetoxy)-2-phenyl-propionyloxy]-9-ethyl-9-methyl-3-oxa-9-azonia-tricyclo[3.3.1.02,4]nonane (Oxitropium-N,N-diethylglycinate); 7-[2-(2-Diethylamino-acetoxy)-2,2-di-thiophen-2-yl-acetoxy]-9,9-dimethyl-3-oxa-9-azonia-tricyclo[3.3.1.02,4]nonane (Tiotropium-N,N-diethylglycinate); Dimethylamino-acetic acid 2-(3-diisopropylamino-1-phenyl-propyl)-4-methyl-phenyl ester (Tolterodine-N,N-dimethylglycinate); 3-[4,4-Bis-(4-fluoro-phenyl)-2-oxo-imidazolidin-1-yl]-1-methyl-1-(2-oxo-2-pyridin-2-yl-ethyl)-pyrrolidinium; 1-[1-(3-Fluoro-benzyl)-piperidin-4-yl]-4,4-bis-(4-fluoro-phenyl)-imidazolidin-2-one; 1-Cyclooctyl-3-(3-methoxy-1-aza-bicyclo[2.2.2]oct-3-yl)-1-phenyl-prop-2-yn-1-ol; 3-[2-(2-Diethylamino-acetoxy)-2,2-di-thiophen-2-yl-acetoxy]-1-(3-phenoxy-propyl)-1-azonia-bicyclo[2.2.2]octane (Aclidinium-N,N-diethylglycinate); or (2-Diethylamino-acetoxy)-di-thiophen-2-yl-acetic acid 1-methyl-1-(2-phenoxy-ethyl)-piperidin-4-yl ester.

The compounds of Formula I-II may also be combined with mucolytic agents to treat both the infection and symptoms of respiratory infections. A non-limiting example of a mucolytic agent is ambroxol. Similarly, the compounds of Formula I-II may be combined with expectorants to treat both the infection and symptoms of respiratory infections. A non-limiting example of an expectorant is guaifenesin.

Nebulized hypertonic saline is used to improve immediate and Ion-term clearance of small airways in patients with lung diseases (Kuzik, J. Pediatrics 2007, 266). The compounds of Formula I-II may also be combined with nebulized hypertonic saline particularly when the Orthomyxoviridae virus infection is complicated with bronchiolitis. The combination of the compounds of Formula I-II with hypertonic saline may also comprise any of the additional agents discussed above. In a preferred aspect, nebulized about 3% hypertonic saline is used.

It is also possible to combine any compound of the invention with one or more other active therapeutic agents in a unitary dosage form for simultaneous or sequential administration to a patient. The combination therapy may be administered as a simultaneous or sequential regimen. When administered sequentially, the combination may be administered in two or more administrations.

Co-administration of a compound of the invention with one or more other active therapeutic agents generally refers to simultaneous or sequential administration of a compound of the invention and one or more other active therapeutic agents, such that therapeutically effective amounts of the compound of the invention and one or more other active therapeutic agents are both present in the body of the patient.

Co-administration includes administration of unit dosages of the compounds of the invention before or after administration of unit dosages of one or more other active therapeutic agents, for example, administration of the compounds of the invention within seconds, minutes, or hours of the administration of one or more other active therapeutic agents. For example, a unit dose of a compound of the invention can be administered first, followed within seconds or minutes by administration of a unit dose of one or more other active therapeutic agents. Alternatively, a unit dose of one or more other therapeutic agents can be administered first, followed by administration of a unit dose of a compound of the invention within seconds or minutes. In some cases, it may be desirable to administer a unit dose of a compound of the invention first, followed, after a period of hours (e.g., 1-12 hours), by administration of a unit dose of one or more other active therapeutic agents. In other cases, it may be desirable to administer a unit dose of one or more other active therapeutic agents first, followed, after a period of hours (e.g., 1-12 hours), by administration of a unit dose of a compound of the invention.

The combination therapy may provide “synergy” and “synergistic”, i.e. the effect achieved when the active ingredients used together is greater than the stun of the effects that results from using the compounds separately. A synergistic effect may be attained when the active ingredients are: (1) co-formulated and administered or delivered simultaneously in a combined formulation; (2) delivered by alternation or in parallel as separate formulations; or (3) by some other regimen. When delivered in alternation therapy, a synergistic effect may be attained when the compounds are administered or delivered sequentially, e.g. in separate tablets, pills or capsules, or by different injections in separate syringes. In general, during alternation therapy, an effective dosage of each active ingredient is administered sequentially, i.e. serially, whereas in combination therapy, effective dosages of two or more active ingredients are administered together. A synergistic anti-viral effect denotes an antiviral effect which is greater than the predicted purely additive effects of the individual compounds of the combination.

### Methods of Treating Patients

In still yet another embodiment, the present application provides for methods of treating Orthomyxoviridae infections in a patient, comprising: administering to the patient a therapeutically effective amount of a compound of Formula I-II, or a pharmaceutically acceptable salt, solvate, and/or ester thereof.

In still yet another embodiment, the present application provides for methods of treating Orthomyxoviridae infections in a patient, comprising: administering to the patient a therapeutically effective amount of a compound of Formula I-II, or a pharmaceutically acceptable salt, solvate, and/or ester thereof, and at least one additional active therapeutic agent, whereby Orthomyxoviridae polymerase is inhibited.

In still yet another embodiment, the present application provides for methods of treating Orthomyxoviridae infections in a patient, comprising: administering to the patient a therapeutically effective amount of a compound of Formula I-II, or a pharmaceutically acceptable salt, solvate, and/or ester thereof, and at least one additional active therapeutic agent selected from the group consisting of interferons, ribavirin analogs, a viral haemagglutinin inhibitor, a viral neuramidase inhibitor, a M2 ion channel blocker, a Orthomyxoviridae RNA-dependent RNA polymerase inhibitor, a sialidase and other drugs for treating Orthomyxoviridae infections.

In still yet another embodiment, the present application provides for the use of a compound of the present invention, or a pharmaceutically acceptable salt, solvate, and/or ester thereof, for the preparation of a medicament for treating an Orthomyxoviridae infections in a patient.

### Metabolites of the Compounds of the Invention

Also falling within the scope of this invention are the in vivo metabolic products of the compounds described herein, to the extent such products are novel and unobvious over the prior art. Such products may result for example from the oxidation, reduction, hydrolysis, amidation, esterification and the like of the administered compound, primarily due to enzymatic processes. Accordingly, the invention includes novel and unobvious compounds produced by a process comprising contacting a compound of this invention with a mammal for a period of time sufficient to yield a metabolic product thereof. Such products typically are identified by preparing a radiolabelled (e.g. ¹⁴C or ³H) compound of the invention, administering it parenterally in a detectable dose (e.g. greater than about 0.5 mg/kg) to an animal such as rat, mouse, guinea pig, monkey, or to man, allowing sufficient time for metabolism to occur (typically about 30 seconds to 30 hours) and isolating its conversion products from the urine, blood or other biological samples. These products are easily isolated since they are labeled (others are isolated by the use of antibodies capable of binding epitopes surviving in the metabolite). The metabolite structures are determined in conventional fashion, e.g. by MS or NMR analysis. In general, analysis of metabolites is done in the same way as conventional drug metabolism studies well-known to those skilled in the art. The conversion products, so long as they are not otherwise found in vivo, are useful in diagnostic assays for therapeutic dosing of the compounds of the invention even if they possess no Orthomyxoviridae polymerase inhibitory activity of their own.

Recipes and methods for determining stability of compounds in surrogate gastrointestinal secretions are known. Compounds are defined herein as stable in the gastrointestinal tract where less than about 50 mole percent of the protected groups are deprotected in surrogate intestinal or gastric juice upon incubation for 1 hour at 37° C. Simply because the compounds are stable to the gastrointestinal tract does not mean that they cannot be hydrolyzed in vivo. The prodrugs of the invention typically will be stable in the digestive system but may be substantially hydrolyzed to the parental drug in the digestive lumen, liver or other metabolic organ, or within cells in general.

### EXAMPLES

Certain abbreviations and acronyms are used in describing the experimental details. Although most of these would be understood by one skilled in the art, Table 1 contains a list of many of these abbreviations and acronyms.

TABLE 1
List of abbreviations and acronyms.

| Abbreviation | Meaning |
|---|---|
| Ac₂O | acetic anhydride |
| AIBN | 2,2′-azobis(2-methylpropionitrile) |
| Bn | Benzyl |
| BnBr | Benzylbromide |
| BSA | bis(trimethylsilyl)acetamide |
| BzCl | benzoyl chloride |
| CDI | carbonyl diimidazole |
| DABCO | 1,4-diazabicyclo[2.2.2]octane |
| DBN | 1,5-diazabicyclo[4.3.0]non-5-ene |
| DDQ | 2,3-dichloro-5,6-dicyano-1,4-benzoquinone |
| DBU | 1,5-diazabicyclo[5.4.0]undec-5-ene |
| DCA | Dichloroacetamide |
| DCC | Dicyclohexylcarbodiimide |
| DCM | Dichloromethane |
| DMAP | 4-dimethylaminopyridine |
| DME | 1,2-dimethoxyethane |
| DMTCl | dimethoxytrityl chloride |
| DMSO | Dimethylsulfoxide |
| DMTr | 4,4′-dimethoxytrityl |
| DMF | Dimethylformamide |
| EtOAc | ethyl acetate |
| ESI | electrospray ionization |
| HMDS | Hexamethyldisilazane |
| HPLC | High pressure liquid chromatography |
| LDA | lithium diisopropylamide |
| LRMS | low resolution mass spectrum |
| MCPBA | meta-chloroperbenzoic acid |
| MeCN | Acetonitrile |
| MeOH | Methanol |
| MMTC | mono methoxytrityl chloride |
| m/z or m/e | mass to charge ratio |
| MH⁺ | mass plus 1 |
| MH⁻ | mass minus 1 |
| MsOH | methanesulfonic acid |
| MS or ms | mass spectrum |
| NBS | N-bromosuccinimide |
| Ph | Phenyl |
| rt or r.t. | room temperature |
| TBAF | tetrabutylammonium fluoride |
| TMSCl | Chlorotrimethylsilane |
| TMSBr | Bromotrimethylsilane |
| TMSI | Iodotrimethylsilane |
| TMSOTf | (trimethylsilyl)trifluoromethylsulfonate |
| TEA | Triethylamine |
| TBA | Tributylamine |
| TBAP | Tributylammonium pyrophosphate |
| TBSCl | t-butyldimethylsilyl chloride |
| TEAB | Triethylammonium bicarbonate |
| TFA | trifluoroacetic acid |
| TLC or tlc | thin layer chromatography |
| Tr | Triphenylmethyl |
| Tol | 4-methylbenzoyl |
| Turbo Grignard | 1:1 mixture of isopropylmagnesium chloride and lithium |
| | chloride |
| δ | parts per million down field from tetramethylsilane |

### Preparation of Compounds

### 2-Deoxy-2-fluoro-4,5-O,O-dibenzyl-D-arabinose

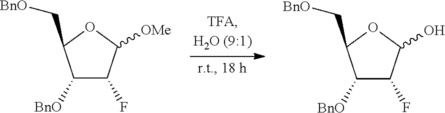 [SMILES: COC1O[C@H](COCc2ccccc2)[C@@H](OCc2ccccc2)[C@H]1F ; OC1O[C@H](COCc2ccccc2)[C@@H](OCc2ccccc2)[C@H]1F]

1′-Methoxy-2-deoxy-2-fluoro-4,5-O,O-dibenzyl-D-arabinose (J. Am Chem. Soc. 127 (31), 2005, 10879) (1.0 g, 2.88 mmol) in TFA (13.5 mL) was treated with H₂O (1.5 mL) and the resultant mixture stirred for 5 h. The mixture was then diluted with EtOAc (100 mL) and treated with saturated NaHCO₃ (50 mL). The organic layer was separated and washed with NaCl (50 mL), dried over anhydrous MgSO₄, filtered and concentrated under reduced pressure. The residue was subjected to silica gel chromatography (80 g SiO₂ Combiflash HP Gold Column) eluting with 0-100% EtOAc in hexanes to afford 2-deoxy-2-fluoro-4,5-O,O-dibenzyl-D-arabinose (695 mg, 72%) as a white solid: R_{f}=0.52 (25% EtOAc in hexanes);

¹H-NMR (300 MHz, CDCl₃) δ 7.30 (m, 10H), 5.35 (m, 1H), 4.68-4.29 (m, 7H), 3.70 (d, J=10.5 Hz, 1H), 3.50 (d, J=10.5 Hz, 2H).

¹⁹F NMR (282.2 MHz, CDCl₃) δ −207 (m), −211 (m).

LCMS m/z 350 [M+H₂O].

### (3R,4R,5R)-4-(benzyloxy)-5-(benzyloxymethyl)-3-fluorodihydrofuran-2(3H)-one

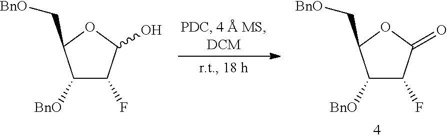 [SMILES: OC1O[C@H](COCc2ccccc2)[C@@H](OCc2ccccc2)[C@H]1F ; O=C1O[C@H](COCc2ccccc2)[C@@H](OCc2ccccc2)[C@H]1F]

2-Deoxy-2-fluoro-4,5-O,O-dibenzyl-D-arabinose (4.3 g, 12.8 mmol) was dissolved in CH₂Cl₂ (85 mL) was treated with 4 Å MS (10 g) and pyridinium dichromate (14.4 g, 38.3 mmol). The resultant mixture was stirred for 24 h and then filtered through a pad of Celite. The eluant was concentrated under reduced pressure and the residue subjected to silica gel chromatography (120 g SiO₂ HP Gold Combiflash Column) eluting with 0-100% EtOAc in hexanes to afford (3R,4R,5R)-4-(benzyloxy)-5-(benzyloxymethyl)-3-fluorodihydrofuran-2(3H)-one (4) as a clear oil (3.5 g, 83%); R_{f}=0.25 (25% EtOAc in hexanes).

¹H NMR (300 MHz, CDCl₃) δ 7.37 (m, 10H), 5.45 (dd, J=49, 5.7, Hz, 1H), 4.85 (d. J=11.7 Hz, 1H), 4.52 (m, 4H), 4.29 (d, J=5.4 Hz, 1H), 2.08 (dd, J=15.3, 10.2 Hz, 2H).

¹⁹F NMR (282.2 MHz, CDCl₃) δ −216.

LCMS m/z 348 [M+H₂O].

HPLC (6-98% MeCN—H₂O gradient, 0.05% TFA modifier) t_{R}=5.29 min.

Phenoinenex Synergi 4 m Hydro-RP 80 A, 50×4.60 mm, 4 micron; 2 mL/min flow rate

### Compound 1: (2R,3R,4R,5S)-5-(4-aminopyrrolo[1,2-f][1,2,4]triazin-7-yl)-4-fluoro-2-(hydroxymethyl)-tetrahydrofuran-3-ol

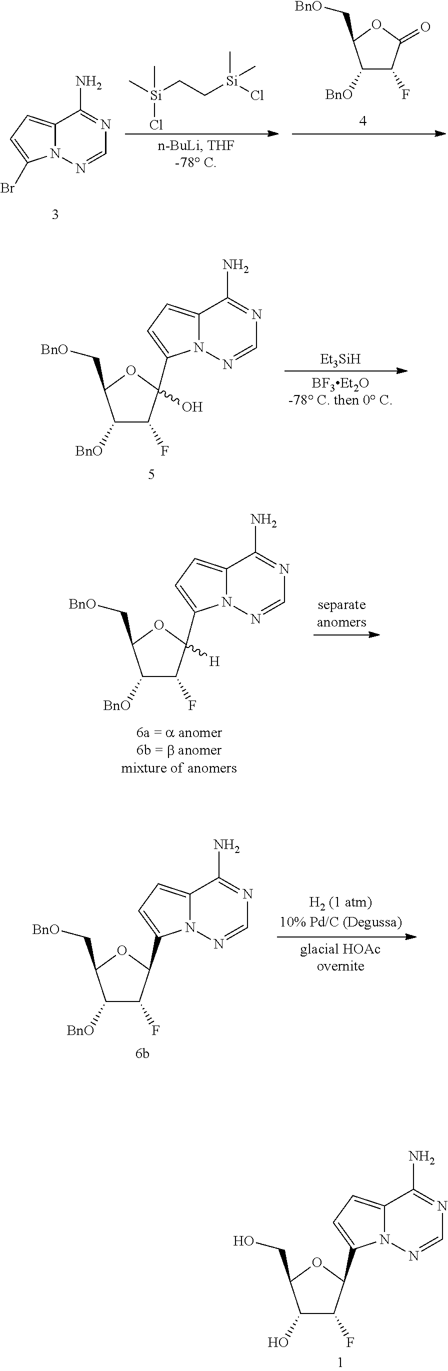

To a suspension of the bromide 3 (prepared according to WO2009/132135) (710 mg, 3.33 mmol) in dry THF (6.0 mL) was added 1,2-bis(chlorodimethylsilyl)ethane (717 mg, 3.33 mmol) in one portion at room temperature. After 1 h, the resulting slurry was cooled to −78° C. and n-BuLi (7.5 mL of a 1.6 M solution in hexanes, 12.0 mmol) was added dropwise over a 5 min period. After stirring for 20 min at this temperature, a solution of 4 (1.0 g, 3.03 mmol) in dry THF (2.85 mL) was added dropwise over several minutes. The reaction was stirred at this temperature for 3 h and then allowed to warm to 0° C. Glacial HOAc (2.5 mL) was added and the reaction was warmed to room temperature. After vigorously stirring for 10 min, the bulk of the solvents were removed under reduced pressure and the reaction mixture was partitioned between ethyl acetate and water. The layers were separated and the organic layer was washed with sat. NaHCO₃, brine, dried over Na₂SO₄ and concentrated to provide a dark brown residue. Purification of the residue by flash column chromatography on silica gel using a gradient of 50% hexanes in ethyl acetate to 20% hexanes in ethyl acetate provided the desired product 5 (591 mg, 42%) as a pale yellow foam.

To a solution of 5 (591 mg, 1.27 mmol) in dry dichloromethane (18.0 mL) cooled to −78° C. was added triethylsilane (0.82 mL, 5.13 mmol) followed by the dropwise addition of BF₃.Et₂O (0.64 mL, 5.13 mmol). After stirring for 4 h, the reaction was warmed to 0° C. and allowed to stir for an additional 30 min. The reaction was diluted with dichloromethane and partitioned between sat. NaHCO₃. The layers were separated and the aqueous layer extracted with dichloromethane. The combined organic layers were dried over Na₂SO₄ and concentrated to provide an orange foam. Purification of the residue by flash column chromatography on silica gel using 20% hexanes in ethyl acetate provided the desired β-anomer 6b (229 mg, 40%) as a yellow foam and a mixture of α- and β-anomers 6ab (110 mg, 19%) as a yellow foam. Rf=0.56 for the α□-anomer and Rf=0.62 for the β-anomer.

To a solution of 6b (66 mg, 0.15 mmol) in glacial HOAc (12 mL) was added 10% palladium on carbon (Degussa type) (70 mg). The reaction was degassed under vacuum and then stirred under an atmosphere of hydrogen gas (via a balloon) overnight. The reaction was filtered through a pad of Celite, washed thoroughly with hot methanol and concentrated in vacuo provided the crude product. Purification of the residue by flash column chromatography on silica gel using 15% methanol in dichloromethane provided the desired product as a solid. The solid was further purified by dissolving in a minimum amount of hot methanol and upon cooling to room temperature the desired product precipitated out. Ethyl ether was added and the product was collected by filtration and washed with ethyl ether. After drying under hi vacuum, the desired product 1 was obtained (16 mg, 41%) as an off-white powder. LC/MS (m/z): 269.2 [M+H]⁺

HPLC retention time: 1.28 min (2-98% acetonitrile:water with 0.05% trifluoroacetic acid).

¹H NMR (400 MHz, DMSO-d₆): δ 7.84 (s, 1H), 7.75 (bs, 2H), 6.82 (d, J=4.4 Hz, 1H), 6.73 (d, J=4.4 Hz, 1H), 5.44 (dd, J=2.4, 23.6 Hz, 1H), 5.01 (ddd, J=2.4, 5.3, 55.1 Hz, 1H), 4.84 (t, J=5.7 Hz, 1H), 4.16-4.06 (m, 1H), 3.82-3.78 (m 1H), 3.69 (ddd, J=2.7, 5.5, 12.1 Hz, 1H) 3.54-3.46 (m, 1H).

¹⁹F (377 MHz, DMSO-d₆): δ −196.36 (dt, J=21.8, 55.1 Hz, 1F).

### (3R,4R,5R)-2-(4-aminopyrrolo[1,2-f][1,2,4]triazin-7-yl)-4-(benzyloxy)-5-(benzyloxymethyl)-3-fluorotetrahydrofuran-2-carbonitrile

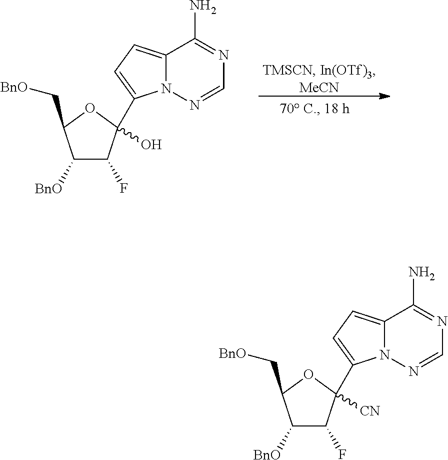

(3R,4R,5R)-2-(4-aminopyrrolo[1,2-f][1,2,4]triazin-7-yl)-4-(benzyloxy)-5-(benzyloxymethyl)-3-fluorotetrahydrofuran-2-ol (5) (195 mg, 0.42 mmol) was dissolved in MeCN (1.4 mL) was treated with TMSCN (336 μL, 2.52 mmol) and In(OTf)₃ (708 mg, 1.26 mmol). The solution was stirred at 70° C. for 18 h and then cooled to 0° C. The mixture was treated with saturated NaHCO₃ solution (20 drops) then warmed to RT and diluted with EtOAc (100 mL) and H₂O (50 mL). The organic layer was separated and washed with saturated NaCl solution (50 mL), dried over MgSO₄, filtered and concentrated under reduced pressure. The residue was subjected to silica gel chromatography (40 g SiO₂ HP Gold Combilash Column) eluting with 0-100% EtOAc in hexanes to afford (3R,4R,5R)-2-(4-aminopyrrolo[1,2-f][1,2,4]triazin-7-yl)-4-(benzyloxy)-5-(benzyloxymethyl)-3-fluorotetrahydrofuran-2-carbonitrile as a white solid (110 mg, 55%, 60/40 mixture of α/β isomers). Data for both isomers: R_{f}=0.53 (EtOAc).

¹H NMR (300 MHz, CDCl₃) δ 8.01 (s, 1H), 7.94 (s, 1H), 7.30 (m, 10H), 7.00 (d, J=4.5 Hz, 1H), 6.93 (d, J=4.8 Hz, 1H), 6.87 (d, J=5.4 Hz, 1H), 6.70 (d, J=4.8 Hz, 1H), 5.85 (dd, J=52, 3.3 Hz, 1H), 5.55 (dd, J=53, 4.5 Hz, 1H), 4.71 (m, 7H), 3.87 (m, 2H), 3.72 (m, 2H).

¹⁹F NMR (282.2 MHz, CDCl₃) δ −196 (m), −203 (m).

LCMS m/z 474 [M+H].

HPLC (6-98% MeCN—H₂O gradient, 0.05% TFA modifier) t_{R}=4.98 min.

### Compound 7: (2R,3R,4R,5R)-2-(4-aminopyrrolo[1,2-f][1,2,4]triazin-7-yl)-3-fluoro-4-hydroxy-5-(hydroxymethyl)tetrahydrofuran-2-carbonitrile (7)

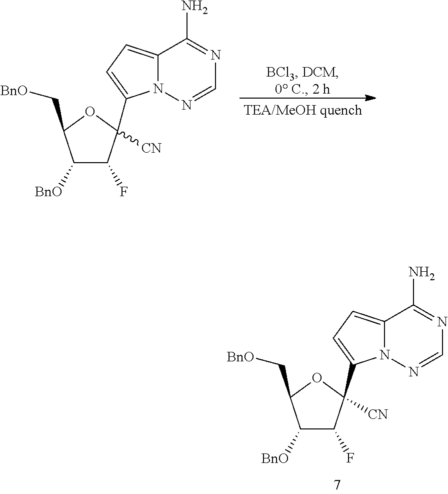 [SMILES: N#CC1(c2ccc3c(N)ncnn23)O[C@H](COCc2ccccc2)[C@@H](OCc2ccccc2)[C@H]1F ; "BCl3, DCM, 0° C., 2 h" ; N#C[C@@]1(c2ccc3c(N)ncnn23)O[C@H](COCc2ccccc2)[C@@H](OCc2ccccc2)[C@H]1F]

(3R,4R,5R)-2-(4-aminopyrrolo[1,2-f][1,2,4]triazin-7-yl)-4-(benzyloxy)-5-(benzyloxymethyl)-3-fluorotetrahydrofuran-2-carbonitrile (110 mg, 0.23 mmol) was dissolved in CH₂Cl₂ (1.5 mL) and cooled to 0 OC. The reaction mixture was treated with BCl₃ (1.0 M in CH₂Cl₂, 766 μL, 0.77 mmol) and stirred for 2 h. The mixture was then cooled to −78° C. and treated with Et₃N (340 μL, 2.44 mmol) followed by MeOH (2 mL) before allowing to warm to RT. The reaction was concentrated under reduced pressure and then co-evaporated with MeOH (3×5 mL). The residue was then suspended in H₂O (5 mL) and treated with NaHCO₃ (1 g). The solution was stirred for 10 min and then concentrated under reduced pressure. The residue was filtered and washed with MeOH (3×10 mL) on a fritted glass funnel (coarse) and the eluant concentrated under reduced pressure. The residue was subjected to reverse phase HPLC (6-98% MeCN in H₂O gradient with 0.05% TFA modifier) to afford (2R,3R,4R,5R)-2-(4-aminopyrrolo[1,2-f][1,2,4]triazin-7-yl)-3-fluoro-4-hydroxy-5-(hydroxymethyl)tetrahydrofuran-2-carbonitrile 7 as a white solid (16.8 mg, 25%) and the α-isomer.

Data for the β-isomer: R_{f}=0.13 (10% MeOH in EtOAc).

¹H NMR (300 MHz, CD₃OD) δ 8.09 (s, 1H), 7.28 (d, J=5.1 Hz, 1H), 7.17 (d, J=5.1 Hz, 1H), 5.42 (dd, J=53, 3.3 Hz, 1H), 4.20 (m, 2H), 3.99 (d. J=3.6 Hz, 1H), 3.77 (d, J=3.6 Hz, 1H).

¹⁹F NMR (282.2 MHz, CDCl₃) δ −197 (m).

LCMS nm/z 294 [M+H].

HPLC (2-98% MeCN—H₂O gradient, 0.05% TFA modifier) t_{R}=1.49 min.

### Compound 8: (2R,3R,4R,5S)-5-(4-aminopyrrolo[1,2-f][1,2,4]triazin-7-yl)-4-fluoro-2-(hydroxymethyl)-5-methyltetrahydrofuran-3-ol

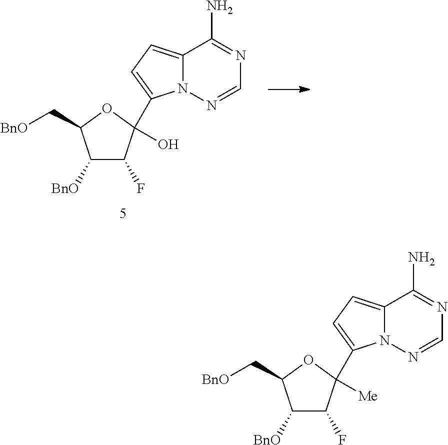 [SMILES: Nc1ncnn2c(C3(O)O[C@H](COCc4ccccc4)[C@@H](OCc4ccccc4)[C@H]3F)ccc12 ; CC1(c2ccc3c(N)ncnn23)O[C@H](COCc2ccccc2)[C@@H](OCc2ccccc2)[C@H]1F]

The starting nucleoside 5 (0.355 g, 0.765 mmol) was dissolved in anhydrous THF (35 mL) and cooled to 0° C. with stirring under N₂ (g). A solution of methyl magnesium chloride (2 mL, 6 mmol) (3N in THF) was added and the resultant mixture stirred overnight. Acetic acid (7 mmol) was added to quench the reaction and then the solvents were removed by rotary under reduced pressure. The residue was re-dissolved in CH₂Cl₂ and the solution subjected to a plug of silica gel to isolate the product (0.355 g) as a crude mixture. LC/MS (m/z: 480. M⁺¹). The crude material was dissolved in anhydrous CH₂Cl₂ (20 mL) and placed under N₂ (g). The solution was stirred and treated with methanesulfonic acid (0.2 mL, 2.74 mmol). The reaction mixture was stirred for 12 h at RT and then quenched by the addition of Et₃N (3.5 mmol). The mixture was concentrated under reduced pressure and the residue subjected to silica gel chromatography to provide the methyl substituted nucleoside (0.174 g, 0.377 mmol, 44% yield) as a 4:1 mixture of beta- and alpha-anomers respectively.

¹H NMR (300 MHz, CD₃CN) major anomer δ 7.87 (s, 1H), 7.27-7.40 (m, 10H), 6.77 (d, J=4.5 HZ, 1H), 6.70 (d, J=4.5 Hz, 1H), 6.23 (br s, 2H), 5.53 (dd, J=55, 3.3 Hz, 1H), 4.42-4.75 (m, 4H), 4.19-4.26 (m, 1H), 3.65-4.00 (m, 3H), 1.74 (d, J=3.9 Hz, 3H).

¹⁹F NMR (282.2 MHz, CD₃CN) major anomer δ −207 (m, 1F)

LCMS m/z 463 [M+H].

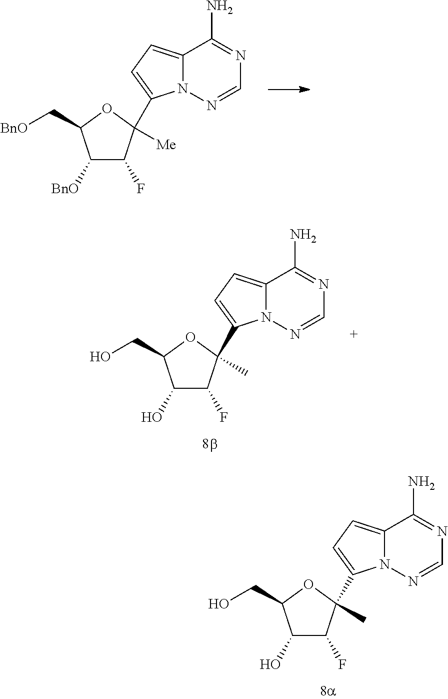 [SMILES: CC1(c2ccc3c(N)ncnn23)O[C@H](COCc2ccccc2)[C@@H](OCc2ccccc2)[C@H]1F ; C[C@@]1(c2ccc3c(N)ncnn23)O[C@H](CO)[C@@H](O)[C@H]1F ; C[C@]1(c2ccc3c(N)ncnn23)O[C@H](CO)[C@@H](O)[C@H]1F]

The benzylated nucleoside material (0.134 g, 0.290 mmol). Degussa catalyst (0.268 g) and AcOH (30 mL) were mixed together. The reaction atmosphere was charged with H₂ (g) and the reaction stirred for 2 h. The catalyst was removed by filtration and the mixture concentrated under reduced pressure. The residue was dissolved in a minimal amount of H₂O and subjected to reverse phase HPLC (C¹⁸ hydro RP column) to isolate the β-anomer (813) (0.086 g, 0.217 mmol, 57% yield).

¹H NMR (300 MHz, D₂O) δ□7.87 (s, 1H), 7.22 (d, J=4.8 Hz, 1H), 6.87 (d, J=4.8 Hz, 1H), 5.35 (dd, J=54, 3.6 Hz, 1H), 3.97-4.10 (m, 2H), 3.81 (dd, J=12.6, 2.1 Hz, 1H), 3.64 (dd, J=12.6, 4.8 Hz, 1H), 1.65 (d, J=4.2 Hz, 3H).

¹⁹F NMR (282.2 MHz, CD₃CN) δ□ −207 (m, 1F).

A small amount of alpha anomer was characterized as follows.

¹H NMR (300 MHz, D₂O) δ□7.86 (s, 1H, 7.26 (d, J=4.8 Hz, 1H), 6.85 (d, J=4.8 Hz, 1H), 5.31 (dd, J=54, 3.9 Hz, 1H), 4.39 (ddd, J=26.1, 9.9, 3.6 Hz, 2H), 4.00-4.05 (m, 1H), 3.90 (dd, J=12.3, 2.1 Hz, 1H), 3.66 (dd, J=12.6, 4.8, 1 H), 1.56 (s, 3H).

¹⁹F NMR (282.2 MHz, CD₃CN) δ□ −198 (dd, J=54, 26 Hz, 1F).

### Compound 9: ((2R,3R,4R,5S)-5-(4-aminopyrrolo[1,2-f][1,2,4]triazin-7-yl)-4-fluoro-3-hydroxy-5-methyltetrahydrofuran-2-yl)methyl tetrahydrogen triphosphate

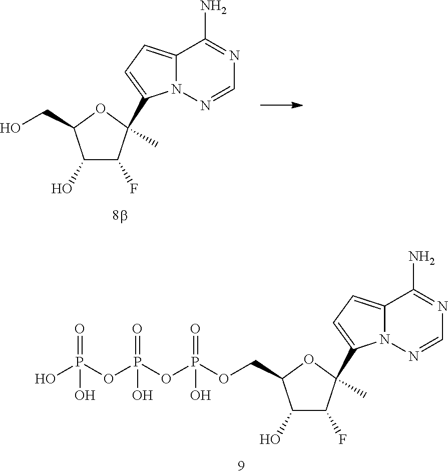 [SMILES: C[C@@]1(c2ccc3c(N)ncnn23)O[C@H](CO)[C@@H](O)[C@H]1F ; C[C@@]1(c2ccc3c(N)ncnn23)O[C@H](COP(=O)(O)OP(=O)(O)OP(=O)(O)O)[C@@H](O)[C@H]1F]

The nucleoside 8β (0.022 g, 0.056 mmol) was dissolved in trimethylphosphate (1 mL) and stirred under N₂ (g). Phosphorous oxychloride (0.067 mL, 0.73 mmol) was added and the mixture stirred for 2 h. Monitoring by analytical ion-exchange column determined the time at which >80 percent of monophosphate was formed. A solution of tributylamine (0.44 mL, 1.85 mmol) and triethylammonium pyrophosphate (0.327 g, 0.72 mmol) dissolved in anhydrous DMF (1 mL) was added. The reaction mixture was stirred for 20 min and then quenched by the addition of 1N triethylammonium bicarbonate solution in H₂O (5 mL). The mixture was concentrated under reduced pressure and the residue re-dissolved in H₂O. The solution was subjected to ion exchange chromatography to yield the title product 9 (1.7 mg, 6% yield).

LCMS m/z 521 [M−H], Tr=0.41

HPLC ion exchange TR=9.40 min

### Compound 10: ((2R,3R,4R,5R)-5-(4-aminopyrrolo[1,2-f][1,2,4]triazin-7-yl-5-cyano-4-fluoro-3-hydroxytetrahydrofuran-2-yl)methyl tetrahydrogen triphosphate

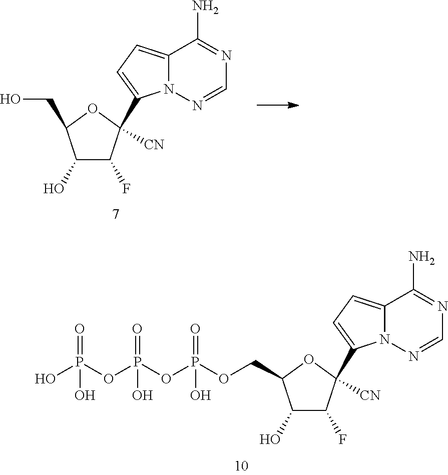 [SMILES: N#C[C@@]1(c2ccc3c(N)ncnn23)O[C@H](CO)[C@@H](O)[C@H]1F ; N#C[C@@]1(c2ccc3c(N)ncnn23)O[C@H](COP(=O)(O)OP(=O)(O)OP(=O)(O)O)[C@@H](O)[C@H]1F]

Compound 10 was prepared from compound 7 using a similar procedure to the preparation of compound 9.

¹H NMR (400 MHz, D₂O) δ 7.78 (s, 1H), 6.93 (d, J=4.4 Hz, 1H), 6.78 (d, J=4.8 Hz, 1H), 5.45 (dd, J=53, 4.4 Hz, 1H), 4.38-4.50 (m, 2H), 4.13-4.20 (m, 2H).

³¹P NMR (161 MHz, D₂O) δ −5.7 (d, 1P), −11.0 (d, 1P), −21.5 (t, 1P).

LCMS m/z 533.9.0 [M+H], 532.0 [M−H] Tr=1.25 min.

HPLC ion exchange Tr-=11.0 min

### Compound 11: ((2R,3R,4R,5S)-5-(4-aminopyrrolo[1,2-f][1,2,4]triazin-7-yl-4-fluoro-3-hydroxy-tetrahydrofuran-2-yl)methyl tetrahydrogen triphosphate

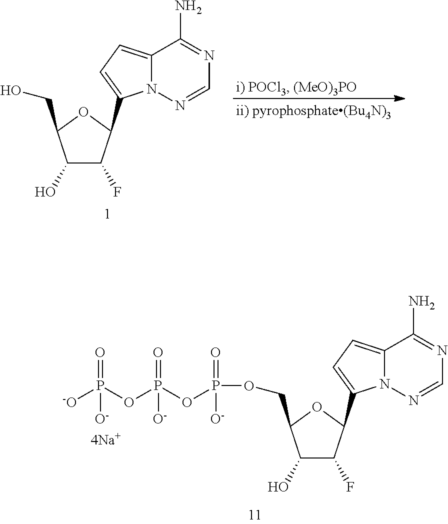

To a solution of nucleoside 1 (21 mg, 0.078 mmol) in trimethyl phosphate (1.0 mL) cooled to 0° C. was added POCl₃ (58 mg, 0.378 mmol) dropwise. The reaction was stirred at 0° C. for 2 h after which, a small aliquot was removed and hydrolyzed with 1.0 M triethylammonium bicarbonate buffer and analyzed by ion exchange HPLC to ensure generation of the nucleoside dichlorophosphoridate. A solution of tris(tetrabutylammonium) hydrogen pyrophosphate (250 mg, 0.277 mmol) and tributylamine (0.15 mL, 0.631 mmol) in dry DMF (1.0 mL) was then added via syringe and the reaction was stirred at 0° C. After 2 h, the reaction was hydrolyzed by the addition of 1.0 M triethylammonium bicarbonate butter (6.0 mL) and the reaction mixture was slowly warmed to room temperature over a period of 1 h. The reaction was concentrated to near dryness under reduced pressure and then co-evaporated from water (×3). The residue was then dissolved in water (10 mL) and lyophilized to give an opaque solid. The solid was dissolved in water (5.0 mL) and purified by ion exchange HPLC. Fractions containing the desired product were pooled and lyophilized to give the desired triphosphate (35 mg) as a colorless solid. Analysis by ³¹P NMR indicated that the material was not of sufficient purity. The solid was dissolved in water (5.0 mL) and stirred with solid NaHCO₃ (50 mg) for 15 min. The water was removed under reduced pressure and the residue was co-evaporated from water (×4) to give a solid that was purified by reverse phase HPLC. Fractions containing the desired product were pooled and evaporated to dryness to provide the desired product 11 (3.5 mg, 7%) as a colorless solid.

¹H NMR (400 MHz, D₂O): δ 7.69 (s, 1H), 6.78 (d, J=4.5 Hz, 1H), 6.74 (d, J=4.5 Hz, 1H), 5.58 (bd, J=24.2 Hz, 1H), 5.11 (bd, J=54.7, 1 H), 4.52-4.40 (m, 1H), 4.20-4.04 (m, 3H).

¹⁹F (377 MHz, D₂O): δ −197.15 (m, J=22.9, 24.1, 55.0 Hz, 1F)

³¹P (162 MHz, D₂O) 8-5.89 (d, J=20.6 Hz, 1P), −10.80 (d, J=19.3 Hz, 1P), −21.80 (apparent t, J=19.3, 20.6 Hz).

### (2S)-ethyl 2-(chloro(phenoxy)phosphorylamino)propanoate (Chloridate A)

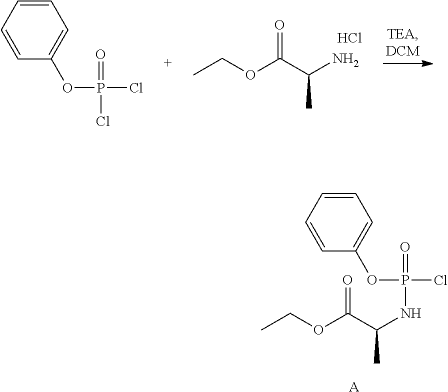

Ethyl alanine ester hydrochloride salt (1.69 g, 11 mmol) was dissolved in anhydrous CH₂C₂ (10 mL) and the mixture stirred with cooling to 0° C. under N₂ (g). Phenyl dichlorophosphate (1.49 mL, 10 mmol) was added followed by dropwise addition of Et₃N over 10 min. The reaction mixture was then slowly warmed to RT and stirred for 12 h. Anhydrous Et₂O (50 mL) was added and the mixture stirred for 30 min. The solid that formed was removed by filtration, and the filtrate concentrated under reduced pressure. The residue was subjected to silica gel chromatography eluting with 0-50% EtOAc in hexanes to provide intermediate A (1.13 g, 39%).

¹H NMR (300 MHz, CDCl₃) δ 7.39-7.27 (m, 5H), 4.27 (m, 3H), 1.52 (m, 3H), 1.32 (m, 3H).

³¹P NMR (121.4 MHz, CDCl₃) δ 8.2, 7.8.

### (2S)-2-ethylbutyl 2-(chloro(phenoxy)phosphorylamino)propanoate (Chloridate B)

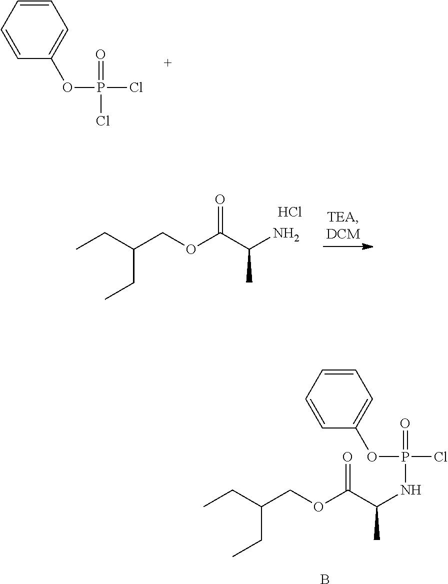

The 2-ethylbutyl alanine chlorophosphoramidate ester B was prepared using the same procedure as chloridate A except substituting 2-ethylbutyl alanine ester for ethyl alanine ester. The material is used crude in the next reaction. Treatment with methanol or ethanol forms the displaced product with the requisite LCMS signal.

### (2S)-isopropyl 2-(chloro(phenoxy)phosphorylamino)propanoate (Chloridate C)

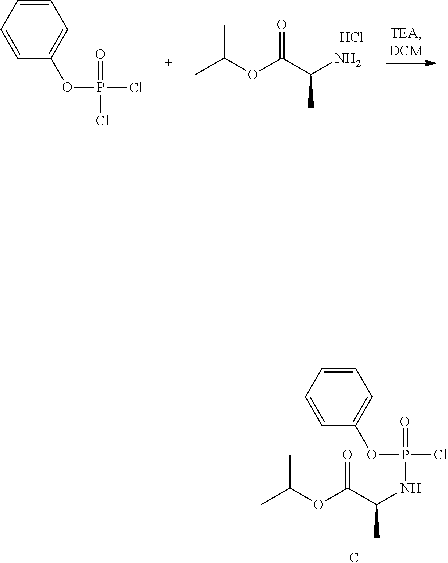

The isopropyl alanine chlorophosphoramidate ester C was prepared using the same procedure as chloridate A except substituting isopropyl alanine ester for the ethyl alanine ester. The material is used crude in the next reaction. Treatment with methanol or ethanol forms the displaced product with the requisite LCMS signal.

### Compound 12: (2R)-isopropyl 2-((((2R,3R,4R,5S)-5-(4-aminopyrrolo[1,2-f][1,2,4]triazin-7-yl)-4-fluoro-3-hydroxy-5-methyltetrahydrofuran-2-yl)methoxy)-(phenoxy)phosphorylamino)propanoate

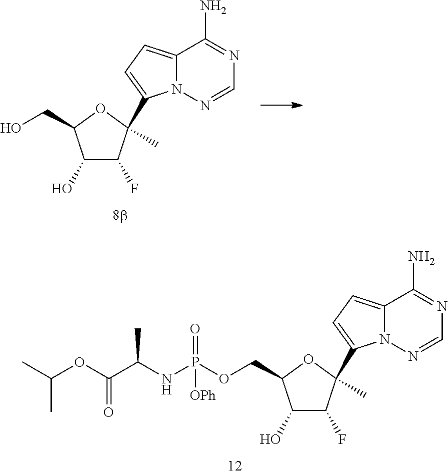 [SMILES: C[C@@]1(c2ccc3c(N)ncnn23)O[C@H](CO)[C@@H](O)[C@H]1F ; CC(C)OC(=O)[C@@H](C)NP(=O)(OC[C@H]1O[C@@](C)(c2ccc3c(N)ncnn23)[C@H](F)[C@@H]1O)Oc1ccccc1]

The nucleoside (0.011 g, 0.04 mmol) was dissolved in trimethylphosphate (2 mL) and cooled to 0° C. The mixture was stirred under an atmosphere of N₂ (g) and 1-Methylimidazole (0.320 mL, 5 mmol) followed by the alaninylmonoisopropyl, monophenol phosphorchloridate C (0.240 mL, 4.4 mmol) was added. The reaction mixture was stirred for 2 h. at 0° C. and then allowed to warm slowly to RT, while monitoring by LC/MS. When complete by LCMS, the reaction mixture was treated with H₂O (5 mL) and then concentrated under reduced pressure. The residue was dissolved in CH₂Cl₂ and subjected to silica gel chromatography eluting with 0-100% EtOAc in hexanes. The product fractions were collected and concentrated. The residue was subjected to prep HPLC to yield the alanine isopropyl monoamidate prodrug 12 as a mixture of isomers (4.7 mg, 0.003 mmol, 6%).

¹H NMR (300 MHz, CD3CN) δ 7.87 (s, 1H), 7.17-7.44 (m, 5H), 6.71-6.83 (m, 2H), 6.14 (br, s, 2H), 5.38 (dd, J=56, 3.3 Hz, 1H), 4.92-5.01 (m, 1H), 3.86-4.46 (m, 6H), 3.58 (m, 1H), 1.73 (m, 3H), 1.18-1.34 (m, 9H)

LCMS m/z 552 [M+H].

### Compound 13: (2R)-ethyl 2-(((2R,3R,4R,5S)-5-(4-aminopyrrolo[1,2-f][1,2,4]triazin-7-yl)-4-fluoro-3-hydroxy-5-methyltetrahydrofuran-2-yl)methoxy)(phenoxy)phosphorylamino)propanoate

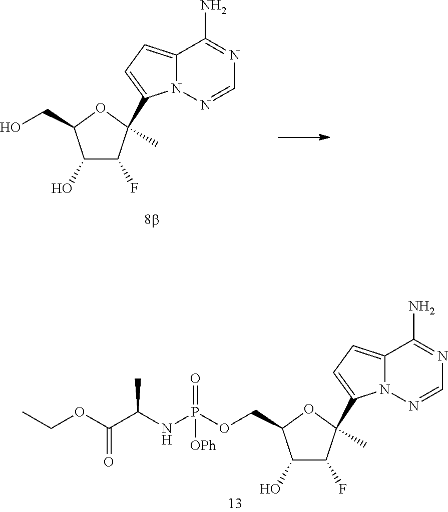 [SMILES: C[C@@]1(c2ccc3c(N)ncnn23)O[C@H](CO)[C@@H](O)[C@H]1F ; CCOC(=O)[C@@H](C)NP(=O)(OC[C@H]1O[C@@](C)(c2ccc3c(N)ncnn23)[C@H](F)[C@@H]1O)Oc1ccccc1]

The nucleoside (0.026 g, 0.092 mmol) was dissolved in trimethylphosphate (2 mL) and cooled to 0° C. The mixture was stirred under N₂ (g) and 1-methylimidazole (0.062 mL, 0.763 mmol) followed by the chloridate A (0.160 g, 0.552 mmol) were added. The reaction mixture was stirred for 2 h. at 0° C. and then allowed to warm slowly to RT. H₂O (5 mL) was added to quench the reaction and then the mixture concentrated under reduced pressure. The residue was dissolved in CH₂Cl₂ and subjected to silica gel chromatography eluting with 0-100% EtOAc in hexanes. The product fractions were collected and concentrated. Crude product was eluted using 0 to 100 percent EtOAc in hexanes. The crude product was collected and concentrated under reduced pressure. The residue was subjected to prep HPLC to yield 13 (2.0 mg, 4% yield).

LCMS m/z 538 [M+H].

### Compound 14: (2S-ethyl 2-(((2R,3R,4R,5R)-5-(4-aminopyrrolo[1,2-f][1,2,4]triazin-7-yl)-5-cyano-4-fluoro-3-hydroxytetrahydrofuran-2-yl)methoxy)(phenoxy)phosphorylamino)propanoate

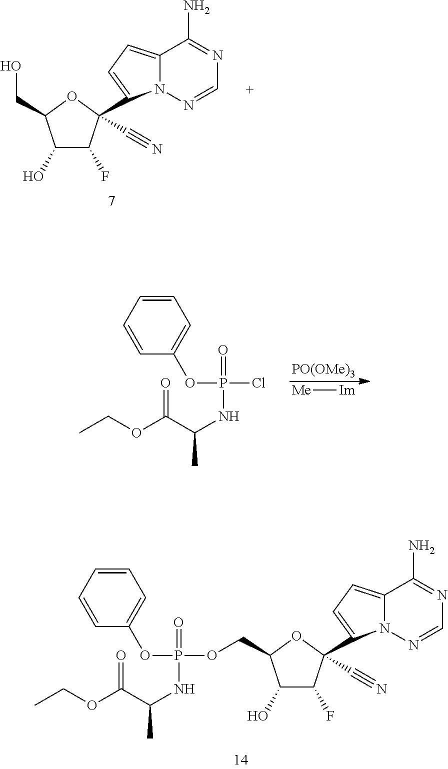

Compound 14 was prepared from Compound 7 and chloridate A using same method as for the preparation of compound 13.

¹H NMR (300 MHz, CD₃OD) δ 7.91 (m, 1H), 7.33-7.16 (m, 5H), 6.98-6.90 (m, 2H), 5.59 (m, 1H), 4.50-4.15 (m, 4H), 4.12-3.90 (m, 3H), 1.33-1.18 (m, 6H).

³¹P NMR (121.4 MHz, CD₃OD) δ 3.8.

LCMS m/z 549.0 [M+H], 547.1 [M−H].

### 7-bromo-2-fluoropyrrolo[1,2-f][1,2,4]triazin-4-amine

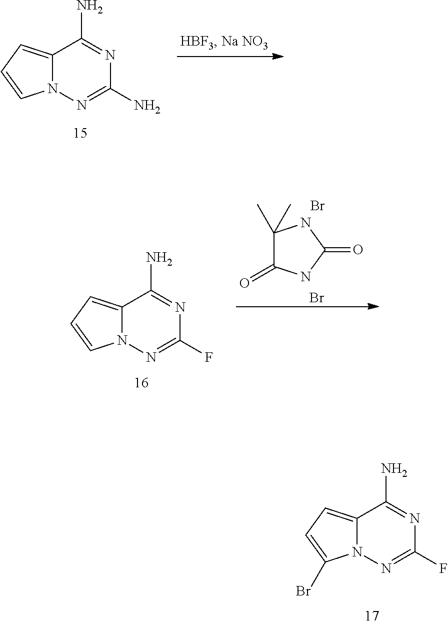

To a solution of 15 (prepared according to WO 2009/132135) (6.0 g, 40.25 mmol) in THF (150 mL) and H₂O (50 mL) at −15° C. was added HBF₄ slowly (36.81 g, 48% by weight in H₂O, 201.24 mmol) over 15 minutes. NaNO₂ (8.33 g, 40% by weight in H₂O, 48.29 mmol) was added to the reaction slowly over 15 minutes. The reaction was stirred at −15° C. for 1 hr. NaOH (200 mL, 1N in H₂O) was added and the solution was allowed to warm to room temperature. The solution was stirred vigorously for 20 minutes. The product was extracted with EtOAc (100 mL×3). The combined organic layers were dried with sodium sulfate, filtered and were concentrated. The product was purified by silica gel chromatography 90%-30% hexanes in ethyl acetate. The product 16 was found to be a yellow solid (1.0 g, 16%).

LC/MS=153 (M+1)

Retention time: 1.55 min

LC: Thermo Electron Surveyor HPLC

MS: Finnigan LCQ Advantage MAX Mass Spectrometer

Column: Phenomenex Polar RP 30 mm×4.6 mm

Solvents: Acetonitrile with 0.1% formic acid, Water with 0.1% formic acid

Gradient: 0 min-0.1 min 5% ACN, 0.1 min-1.95 min 5%-100% ACN, 1.95 min-3.5 min 100% ACN, 3.5 min-3.55 min 100%-5% ACN, 3.55 min-4 min 5% ACN.

To a solution of 16 (1.2 g, 7.8 mmol) in DMF (50 mL) at 0° C. under an atmosphere of argon was added a solution of 1,3-dibromo-5,5-dimethylhydantoin (1.35 g, 4.7 mmol) in DMF (50 mL) dropwise over 30 minutes. The reaction was stirred at 0° C. for 15 minutes. A saturated aqueous solution of Na₂SO₄ (50 mL) and H₂O (50 mL) were added and allowed to warm to room temperature. The reaction was extracted with ethyl acetate (50 mL×3). The combined organics were dried with sodium sulfate, filtered and were concentrated. The product was purified by silica gel chromatography 100% to 50% hexanes in ethyl acetate to yield 17 (712 mg, 40%) as an off-white solid.

¹H NMR (400 MHz, DMSO-d₆): δ 8.50 (d, J=17.5 Hz, 1H), 7.10 (d, J=4.5, 1H), 6.78 (d, J=4.5, 1H).

### Compound 20: (2R,3R,4R,5S)-5-(4-amino-2-fluoropyrrolo[1,2-f][1,2,4]triazin-7-yl)-4-fluoro-2-(hydroxymethyl)tetrahydrofuran-3-ol

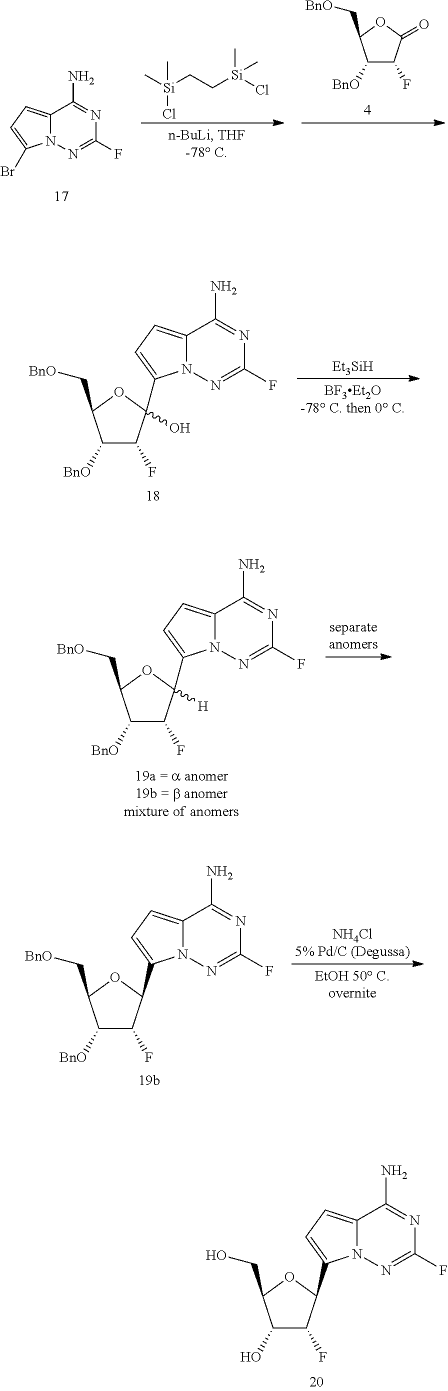

To a suspension of the bromide 17 (400) mg, 1.73 mmol) in dry THF (5.0 mL) was added 1,2-bis(chlorodimethylsilyl)ethane (372 mg, 1.73 mmol) in one portion at room temperature. After 1 h, the resulting slurry was cooled to −78° C. and n-BuLi (3.26 mL of a 1.6 M solution in hexanes, 5.22 mmol) was added dropwise over a 5 min period. After stirring for 20 min at this temperature, a solution of 4 (2.86 mg, 0.87 mmol) in dry THF (2.0 mL) was added dropwise over several minutes. The reaction was stirred at this temperature for 30 min and then allowed to warm to 0° C. An saturated solution of aqueous ammonium chloride (10.0 mL) was added and the reaction was warmed to room temperature. After vigorously stirring for 10 min, the bulk of the solvents were removed under reduced pressure and the reaction mixture was partitioned between ethyl acetate and water. The layers were separated and the organic layer was washed with sat. NaHCO₃, brine, dried over Na₂SO₄ and concentrated to provide a dark brown residue. Purification of the residue by flash column chromatography on silica gel using a gradient of 100% hexanes in ethyl acetate to 50% hexanes in ethyl acetate provided the desired product 18 (287 mg, 68%).

LC/MS=465 (M−17)

Retention time: 2.24 min

LC: Thermo Electron Surveyor HPLC

MS: Finnigan LCQ Advantage MAX Mass Spectrometer

Column: Phenomenex Polar RP 30 mm×4.6 mm

Solvents: Acetonitrile with 0.1% formic acid. Water with 0.1% formic acid

Gradient: 0 min-0.1 min 5% ACN, 0.1 min-1.95 min 5%-100% ACN, 1.95 min-3.5 min 100% ACN, 3.5 min-3.55 min 100%-5% ACN, 3.55 min-4 min 5% ACN.

To a solution of 18 (304 mg, 0.63 mmol) in dry dichloromethane (3.0 mL) cooled to 0° C. was added triethylsilane (0.81 mL, 5.05 mmol) followed by the dropwise addition of BF₃.Et₂O (0.62 mL, 5.05 mmol). After stirring for 20 min, the reaction was warmed to 20° C. and allowed to stir for an additional 30 min. The reaction was diluted with dichloromethane and partitioned between sat. NaHCO₃. The layers were separated and the aqueous layer extracted with dichloromethane. The combined organic layers were dried over Na₂SO₄ and concentrated. Purification of the residue by flash column chromatography on silica gel using 70% hexanes in ethyl acetate provided the desired β-anomer 19b (110 mg, 37%).

LC/MS=467 (M+1)

Retention time: 2.55 min

LC: Thermo Electron Surveyor HPLC

MS: Finnigan LCQ Advantage MAX Mass Spectrometer

Column: Phenomenex Polar RP 30 mm×4.6 mm

Solvents: Acetonitrile with 0.1% formic acid, Water with 0.1% formic acid

Gradient: 0 min-0.1 min 5% ACN, 0.1 min-1.95 min 5%-100% ACN, 1.95 min-3.5 min 100% ACN, 3.5 min-3.55 min 100%-5% ACN, 3.55 min-4 min 5% ACN.

To a solution of 19b (110 mg, 0.24 mmol) in EtOH (3 mL) was added 5% palladium on carbon (Degussa type) (55 mg) and NH₄Cl (128 mg, 2.4 mmol) in a sealed tube. The reaction was degassed under vacuum and then stirred under an atmosphere of argon gas overnight. The reaction was filtered through a pad of Celite, washed thoroughly with methanol and concentrated in vacuo provided the crude product. Purification of the residue by HPLC using 25% ACN in water provided the desired product as a solid. The desired product 20 was obtained (25 mg, 36%) as an off-white powder.

LC/MS=287 (M−1)

Retention time: 1.31-1.38 min

LC: Thermo Electron Surveyor HPLC

MS: Finnigan LCQ Advantage MAX Mass Spectrometer

Column: Phenomenex Polar RP 30 mm×4.6 mm

Solvents: Acetonitrile with 0.1% formic acid, Water with 0.1% formic acid

Gradient: 0 min-0.1 min 5% ACN, 0.1 min-1.95 min 5%-100% ACN, 1.95 min-3.5 min 100% ACN, 3.5 min-3.55 min 100%-5% ACN, 3.55 min-4 min 5% ACN.

¹H NMR (400 MHz, CD₃OD): δ 6.90 (d. J=3.5 Hz, 1H), 6.74 (d, J=3.5, 1H), 5.48 (dd, J=24.0, 2.3 Hz, 1H), 5.10 (dm, J=52.8 Hz, 1H), 4.35-4.26 (m, 1H), 4.0-3.97 (m, 1H), 3.90 (dd, J=12.4, 2.5 Hz, 1H), 3.72 (dd, J=12.4, 4.7 Hz, 1H).

¹⁹F (376 MHz, CD3OD): δ −198.80-−199.3 (m, 1F)

### Compound 21: ((2R,3R,4R,5S)-5-(4-amino-2-fluoropyrrolo[1,2-f][1,2,4]triazin-7-yl)-4-fluoro-3-hydroxytetrahydrofuran-2-yl)methyl tetrahydrogen triphosphate

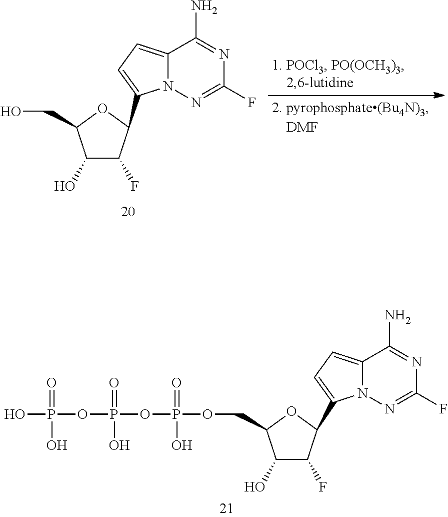 [SMILES: Nc1nc(F)nn2c([C@@H]3O[C@H](CO)[C@@H](O)[C@H]3F)ccc12 ; Nc1nc(F)nn2c([C@@H]3O[C@H](COP(=O)(O)OP(=O)(O)OP(=O)(O)O)[C@@H](O)[C@H]3F)ccc12]

To a solution of nucleoside 20 (7.2 mg, 0.025 mmol) in trimethyl phosphate (0.4 mL) cooled to 0° C. was added POCl₃ (25 mg, 0.151 mmol) dropwise. The reaction was stirred at 0° C. for 30 min, 2,6-lutidine (5 mg, 0.05 mmol) was added dropwise. The reaction was stirred at 0° C. for another 30 min after which, a small aliquot was removed and hydrolyzed with 1.0 M triethylammonium bicarbonate buffer and analyzed by ion exchange HPLC to ensure generation of the nucleoside dichlorophosphoridate. A solution of tris(tetrabutylammonium) hydrogen pyrophosphate (250 mg, 0.277 mmol) and tributylamine (0.15 mL, 0.631 mmol) in dry DMF (1.0 mL) was then added via syringe and the reaction was stirred at 0° C. After 2 h, the reaction was hydrolyzed by the addition of 1.0 M triethylammonium bicarbonate buffer (6.0 mL) and the reaction mixture was slowly warmed to room temperature over a period of 1 h. The reaction was concentrated to near dryness under reduced pressure and then co-evaporated from water (×4). The solid was dissolved in water (5.0 mL) and purified by ion exchange HPLC. Fractions containing the desired product were pooled and concentrated to give the desired triphosphate as a colorless solid. Analysis by ³¹P NMR indicated that the material was not of sufficient purity. The solid was dissolved in water and purified by reverse phase HPLC (Mobile phase A: 10 mM triethylammoniumbicarbonate/AcOH (pH=7), Mobile phase B: CH₃CN) to give the pure triphosphate 21 as a colorless solid (3.1 mg, the amount was calculated based on the analytical HPLC using the parent nucleoside as reference).

LC/MS (m/z): 525.0 [M−H]

³¹P (162 MHz, D₂O) δ −10.42 (d, J=18.0 Hz, 1P), −11.15 (d, J=19.3 Hz, 1P), −23.09 (broad, 1P).

### Antiviral Activity

Another aspect of the invention relates to methods of inhibiting viral infections, comprising the step of treating a sample or subject suspected of needing such inhibition with a composition of the invention.

Within the context of the invention samples suspected of containing a virus include natural or man-made materials such as living organisms; tissue or cell cultures; biological samples such as biological material samples (blood, serum, urine, cerebrospinal fluid, tears, sputum, saliva, tissue samples, and the like); laboratory samples; food, water, or air samples, bioproduct samples such as extracts of cells, particularly recombinant cells synthesizing a desired glycoprotein; and the like. Typically the sample will be suspected of containing an organism which induces a viral infection, frequently a pathogenic organism such as a tumor virus. Samples can be contained in any medium including water and organic solvent\water mixtures. Samples include living organisms such as humans, and man made materials such as cell cultures.

If desired, the anti-virus activity of a compound of the invention after application of the composition can be observed by any method including direct and indirect methods of detecting such activity. Quantitative, qualitative, and semiquantitative methods of determining such activity are all contemplated. Typically one of the screening methods described above are applied, however, any other method such as observation of the physiological properties of a living organism are also applicable.

The antiviral activity of a compound of the invention can be measured using standard screening protocols that are known. For example, the antiviral activity of a compound can be measured using the following general protocols.

### Anti-Influenza Assays

### Influenza A Antiviral and Cytotoxicity Assays (H3N2)

MDCK cells were seeded in 96-well plates at a density of 1e5 cells per well in 100 μL of MEM culture medium with 10% FBS. Compounds were 3-fold serially diluted in complete MEM culture medium, with 100 μM as the highest concentration. Each concentration was tested in duplicate. Prior to infection, cells were washed once with 200 μL serum-free MEM. Influenza A virus (A/Hong Kong/8/68, Advanced Biotechnology Inc, Columbia, Md.) was added to cells at MOI 0.03 in 100 ul serum-free MEM containing 27 U/mL trypsin (Worthington, Lakewood, N.J.). After 10 minute incubation at room temperature, 100 μL compound dilutions were added to infected cells for a final volume of 200 μL. After five day incubation at 37° C., virus-induced cytopathic effect was determined by adding Cell-titer Glo viability reagents (Promega, Madison, Wis.) and measuring luminescence on a Victor Luminescence plate reader (Perkin-Elmer, Waltham, Mass.). The cytotoxicity of the compounds in MDCK cells was determined in replicate plates in the same way as in antiviral activity assays, except no virus was added to the cell culture. EC₅₀ and CC₅₀ values were calculated by non-linear regression of multiple data sets using XLFit software (IBDS, Guildford, UK).

Using this protocol, Compound 1 had an EC₅₀ of about 10.5-12.7 μM against the influenza virus.

### Influenza RNA Polymerase Inhibition (IC50) Assay

Influenza A/PR8/34 (H1N1) purified virus was obtained from Advanced Biotechnologies Inc. (Columbia, Md.) as suspension in PBS buffer. Virions were disrupted by exposure to an equal volume of 2% Triton X-100 for 30 minutes at room temperature in a buffer containing 100 mM Tris-HCl, pH 8, 200 mM KCl, 3 mM dithiothreitol [DTT], 10% glycerol, 10 mM MgCl₂, 2 U/mL RNasin Ribonuclease Inhibitor, and 2 mg/mL Lysolechithin type V (Sigma, Saint Louis, Mo.). The virus lysate was stored at −80° C. in aliquots.

The concentrations refer to final concentrations unless mentioned otherwise.

Nucleotide analog inhibitors were serially diluted 3 fold in water and added to reaction mix containing 10% virus lysate (v/v), 100 mM Tris-HCl (pH 8.0), 100 mM KCl, 1 mM DTT, 10% glycerol, 0.25% Triton-101 (reduced), 5 mM MgC₂, 0.4 U/mL RNasin, and 200 μM ApG dinucleotide primer (TriLink, San Diego Calif.). Reactions were initiated by addition of ribonucleotide triphosphate (NTP) substrate mix containing one α-³³P labeled NTP and 100 μM of the other three natural NTPs (PerkinElmer, Shelton, Conn.). The radiolabel used for each assay matched the class of nucleotide analog screened. The concentrations for the limiting natural NTP are 20, 10, 2, and 1 μM for ATP, CTP, UTP, and GTP respectively. The molar ratio of un-radiolabeled:radiolabeled NTP were in the range of 100-400:1.

Reactions were incubated at 30° C. for 90 minutes then spotted onto DE81 filter paper. Filters were air dried, washed 0.125 M Na₂HPO₄ (3×), water (1×), and EtOH (1×), and air dried before exposed to Typhoon phosphor imager and radioactivity was quantified on a Typhoon Trio (GE Healthcare, Piscataway N.J.). IC₅₀ values were calculated for inhibitors by fitting the data in GraphPad Prism with a sigmoidal dose response with variable slope equation, fixing the Ymax and Ymin values at 100% and 0%.

Using this protocol, Compound 11 had an IC₅₀ of 0.95-1.59 μM. Compound 9 had an IC₅₀ of 2.1-2.97 μM, Compound 10 had an IC₅₀ of 48.6-116 μM, and Compound 21 had an IC₅₀ of 0.97-1.87 μM.

All publications, patents, and patent documents cited herein above are incorporated by reference herein, as though individually incorporated by reference.

The invention has been described with reference to various specific and preferred embodiments and techniques. However, one skilled in the art will understand that many variations and modifications may be made while remaining within the spirit and scope of the invention.

What is claimed is:

1. A method for treating a Orthomyxoviridae infection in a mammal in need thereof comprising administering a therapeutically effective amount of a compound of Formula I: 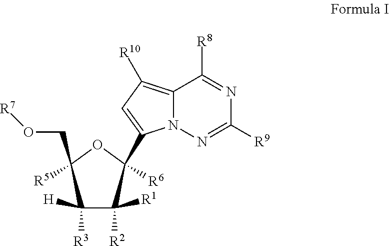
or a pharmaceutically acceptable salt or ester, thereof;
wherein:
each R¹ is H or halogen;
each R² is halogen;
each R³ or R⁵ is independently H, OR^{a}, N(R^{a})₂, N₃, CN, NO₂, S(O)ₙR^{a}, halogen, (C₁-C₈)alkyl, (C₄-C₈)carbocyclylalkyl, (C₁-C₈)substituted alkyl, (C₂-C₈)alkenyl, (C₂-C₈)substituted alkenyl, (C₂-C₈)alkynyl or (C₂-C₈)substituted alkynyl;
R⁶ is H, OR^{a}, N(R^{a})₂—, N₃, CN, NO₂, S(O)ₙR^{a}, —C(═O)R¹¹, —C(═O)OR¹¹, —C(═O)NR¹¹R¹², —C(═O)SR¹¹, —S(O)R¹¹, —S(O)₂R¹¹, —S(O)(OR¹¹), —S(O)₂(OR¹¹), —SO₂NR¹¹R¹², halogen, (C₁-C₈)alkyl, (C₄-C₈)carbocyclylalkyl, (C₁-C₈)substituted alkyl, (C₂-C₈)alkenyl, (C₂-C₈)substituted alkenyl, (C₂-C₈)alkynyl, (C₂-C₈)substituted alkynyl, or aryl(C₁-C₈)alkyl;
each n is independently 0, 1, or 2;
each R^{a} is independently H, (C₁-C₈)alkyl, (C₂-C₈)alkenyl, (C₂-C₈)alkynyl, aryl(C₁-C₈)alkyl, (C₄-C₈)carbocyclylalkyl, —C(═O)R¹¹, —C(═O)OR¹¹, —C(═O)N¹¹R¹², —C(═O)SR¹¹, —S(O)R¹¹, —S(O)₂R¹¹, —S(O)(OR¹¹), —S(O)₂(OR¹¹), or —SO₂NR¹¹R¹²;
R⁷ is H, —C(═O)R, —C(═O)OR¹¹, —C(═O)NR¹¹R¹², —C(═O)SR¹¹, —S(O)R¹¹, —S(O)₂R¹¹, —S(O)(OR¹¹), —S(O)₂(OR¹¹), —SO₂NR¹¹R¹², or 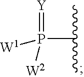
each Y or Y¹ is, independently, O, S, NR, ⁺N(O)(R), N(OR), ⁺N(O)(OR), or N—NR₂;
W¹ and W², when taken together, are —Y³(C(R^{y})₂)₃Y³—; or one of W¹ or W² together with either R³ is —Y³— and the other of W¹ or W² is Formula Ia; or W¹ and W² are each, independently, a group of the Formula Ia: 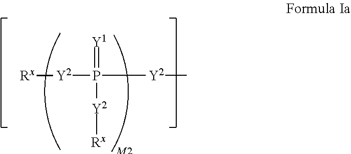
wherein:
each Y² is independently a bond, O, CR₂, NR, ⁺N(O)(R), N(OR), ⁺N(O)(OR), N—NR₂, S, S—S, S(O), or S(O)₂;
each Y³ is independently O, S, or NR;
M2 is 0, 1 or 2;
each R^{x} is independently R^{y} or the formula: 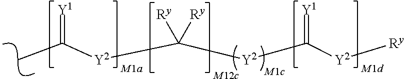
wherein:
each M1a, M1c, and M1d is independently 0 or 1;
M12c is 0, 1, 2, 3, 4, 5, 6, 7, 8, 9, 10, 11 or 12;
each R^{y} is independently H, F, Cl, Br, I, OH, R, —C(═Y¹)R, —C(═Y¹)OR, —C(═Y¹)N(R)₂, —N(R)₂, —⁺N(R)₃, —SR, —S(O)R, —S(O)₂R, —S(O)(OR), —S(O)₂(OR), —OC(═Y¹)R, —OC(═Y¹)OR, —OC(═Y¹)(N(R)₂), —SC(═Y¹)R, —SC(═Y¹)OR, —SC(═Y¹)(N(R)₂), —N(R)C(═Y¹)R, —N(R)C(═Y¹)OR, —N(R)C(═Y¹)N(R)₂, —SO₂NR₂, —CN, —N₃, —NO₂, —OR, or W³; or when taken together, two R^{y} on the same carbon atom form a carbocyclic ring of 3 to 7 carbon atoms;
each R is independently H, (C₁-C₈) alkyl, (C₁-C₈) substituted alkyl, (C₂-C₈)alkenyl, (C₂-C₈) substituted alkenyl, (C₂-C₈) alkynyl, (C₂-C₈) substituted alkynyl, C₆-C₂₀ aryl, C₆-C₂₀ substituted aryl, C₂-C₂₀ heterocyclyl, C₂-C₂₀ substituted heterocyclyl, arylalkyl or substituted arylalkyl;
W³ is W⁴ or W⁵; W⁴ is R, —C(Y¹)R^{y}, —C(Y¹)W⁵, —SO₂R^{y}, or —SO₂W⁵; and W⁵ is a carbocycle or a heterocycle wherein W⁵ is independently substituted with 0 to 3 R^{y} groups;
each R⁸ is halogen, NR¹¹R¹², N(R¹¹)OR¹¹, NR¹¹NR¹¹R¹², N₃, NO, NO₂, CHO, CN, —CH(═NR¹¹), —CH═NNHR¹¹, —CH═N(OR¹¹), —CH(OR¹¹)₂, —C(═O)NR¹¹R¹², —C(═S)NR¹¹R¹², —C(═O)OR¹¹, (C₁-C₈)alkyl, (C₂-C₈)alkenyl, (C₂-C₈)alkynyl, (C₄-C₈)carbocyclylalkyl, optionally substituted aryl, optionally substituted heteroaryl, —C(═O)(C₁-C₈)alkyl, —S(O)ₙ(C₁-C₈)alkyl, aryl(C₁-C₈)alkyl, OR¹¹ or SR¹¹;
each R⁹ or R¹⁰ is independently H, halogen, NR¹¹R¹², N(R¹¹)OR¹¹, NR¹¹NR¹¹R¹², N₃, NO, NO₂, CHO, CN, —CH(═NR¹¹), —CH═NHNR¹¹, —CH═N(OR¹¹), —CH(OR¹¹)₂, —C(═O)NR¹¹R¹², —C(═S)NR¹¹R¹², —C(═O)OR¹¹, R¹¹, OR¹¹ or SR¹¹;
each R¹¹ or R¹² is independently H, (C₁-C₈)alkyl, (C₂-C₈)alkenyl, (C₂-C₈)alkynyl, (C₄-C₈)carbocyclylalkyl, optionally substituted aryl, optionally substituted heteroaryl, —C(═O)(C₁-C₈)alkyl, —S(O)(C₁-C₈)alkyl or aryl(C₁-C₈)alkyl; or R¹¹ and R¹² taken together with a nitrogen to which they are both attached form a 3 to 7 membered heterocyclic ring wherein any one carbon atom of said heterocyclic ring can optionally be replaced with —O—, —S— or —NR^{a}—; and
wherein each (C₁-C₈)alkyl, (C₂-C₈)alkenyl, (C₂-C₈)alkynyl or aryl(C₁-C₈)alkyl of each R³, R⁵, R⁶, R¹¹ or R¹² is, independently, optionally substituted with one or more halo, hydroxy, CN, N₃, N(R^{a})₂ or OR^{a}; and wherein one or more of the non-terminal carbon atoms of each said (C₁-C₈)alkyl may be optionally replaced with —O—, —S— or —NR^{a}—.

2. The method of claim 1 wherein the compound of Formula I represented by Formula II: 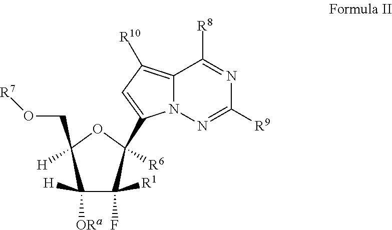
or a pharmaceutically acceptable salt or ester, thereof;
wherein the variables are defined as for Formula I.

3. The method of claim 1 wherein:
R⁷ is H, —C(═O)R¹¹, —C(═O)OR¹¹, —C(═O)NR¹¹R¹², —C(═O)SR¹¹, —S(O)R¹¹, —S(O)₂R¹¹, —S(O)(OR¹¹), —S(O)₂(OR¹¹), —SO₂NR¹¹R¹², or 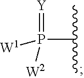
Y is O, S, NR, ⁺N(O)(R), N(OR), ⁺N(O)(OR), or N—NR²;
W¹ and W², when taken together, are —Y³(C(R^{y})₂)₃Y³—; or one of W¹ or W² together with either R³ or R⁴ is —Y³— and the other of W¹ or W² is Formula Ia; or W¹ and W² are each, independently, a group of the Formula Ia: 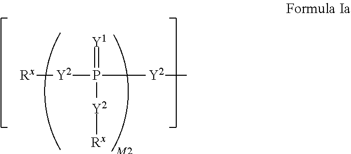
wherein:
each Y¹ is, independently, O, S, NR, ⁺N(O)(R), N(OR), ⁺N(O)XOR), or N—NR₂;
each Y² is independently a bond, O, CR₂, NR, ⁺N(O)(R), N(OR), ⁺N(O)(OR), N—NR₂, S, S—S, S(O), or S(O)₂;
each Y³ is independently O, S, or NR;
M2 is 0, 1 or 2;
each R^{x} is a group of the formula: 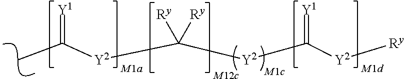
wherein:
each M1a, M1c, and M1d is independently 0 or 1;
M12c is 0, 1, 2, 3, 4, 5, 6, 7, 8, 9, 10, 11 or 12;
each R^{y} is independently H, F, Cl, Br, I, OH, —CN, —N₃, —NO₂, —OR, —C(═Y¹)R, —C(═Y¹)W⁵, —C(═Y¹)OR, —C(═Y¹)N(R)₂, —N(R)₂, —⁺N(R)₃, —SR, —S(O)R, —S(O)₂R, —S(O)₂W⁵, —S(O)(OR), —S(O)₂(OR), —OC(═Y¹)R, —OC(═Y¹)OR, —OC(═Y¹)(N(R)₂), —SC(═Y¹)R, —SC(═Y¹)OR, —SC(═Y¹)(N(R)₂), —N(R)C(═Y¹)R, —N(R)C(═Y¹)OR, —N(R)C(═Y¹)N(R)₂, —SO₂NR₂, (C₁-C₈) alkyl, (C₂-C₈)alkenyl, (C₂-C₈) alkynyl, C₆-C₂₀ aryl, C₃-C₂₀ carbocycle, 3-20 membered heterocyclyl, or arylalkyl; wherein each alkyl, alkenyl, alkynyl, aryl, heterocyclyl, or arylalkyl is independently optionally substituted with one or more Z groups, and each carbocycle is independently optionally substituted with one to three R^{w} groups;
or when taken together, two R^{y} on the same carbon atom form a carbocyclic ring of 3 to 7 carbon atoms;
each W⁵ is independently a carbocycle or a heterocycle optionally substituted with 1 to 3 R^{z} groups;
each R^{w} is independently F, Cl, Br, I, OH, —CN, —N₃, —NO₂, —OR, —C(═Y¹)R, —C(═Y¹)OR, —C(═Y¹)N(R)₂, —N(R)₂, —⁺N(R)₃, —SR, —S(O)R, —S(O)₂R, —S(O)(OR), —S(O)₂(OR), —OC(═Y¹)R, —OC(═Y¹)OR, —OC(═Y¹)(N(R)₂), —SC(═Y¹)R, —SC(═Y¹)OR, —SC(═Y¹)(N(R)₂), —N(R)C(═Y¹)R, —N(R)C(═Y¹)OR, —N(R)C(═Y¹)N(R)₂, —SO₂NR₂, (C₁-C₈) alkyl, (C₂-C₈)alkenyl, (C₂-C₈) alkynyl, C₅-C₂₀ aryl, 3-20 membered heterocyclyl, or arylalkyl; wherein each alkyl, alkenyl, alkynyl, aryl, heterocyclyl, or arylalkyl is independently optionally substituted with one or more Z groups, and each carbocycle is optionally substituted with one to three R^{z} groups;
each R^{z} is independently F, Cl, Br, I, OH, —CN, —N₃, —NO₂, —OR, —C(═Y¹)R, C(═Y¹)W⁵, —C(═Y¹)OR, —C(═Y¹)N(R)₂, —N(R)_{2,} ⁺N(R)₃, —SR, —S(O)R, —S(O)₂R, —S(O)₂W⁵, —S(O)(OR), —S(O)₂(OR), —OC(═Y¹)R, —OC(═Y¹)OR, —OC(═Y¹)(N(R)₂), —SC(═Y¹)R, —SC(═Y¹)OR, —SC(═Y¹)(N(R)₂), —N(R)C(═Y¹)R, —N(R)C(═Y¹)OR, —N(R)C(═Y¹)N(R)₂, or —SO₂NR₂;
each R is independently H, (C₁-C₈) alkyl, (C₂-C₈)alkenyl, (C₂-C₈) alkynyl, C₆-C₂₀ aryl, C₂-C₂₀ heterocyclyl, or arylalkyl; wherein each alkyl, alkenyl, alkynyl, aryl, heterocyclyl, or arylalkyl is independently optionally substituted with one or more Z groups;
each R¹¹ or R¹² is independently H, (C₁-C₈)alkyl, (C₂-C₈)alkenyl, (C₂-C₈)alkynyl, (C₄-C₈)carbocyclylalkyl, aryl, heteroaryl, —C(═O)(C₁-C₈)alkyl, —S(O)ₙ(C₁-C₈)alkyl or aryl(C₁-C₈)alkyl; wherein each aryl or heteroaryl is independently optionally substituted with one or more Z groups;
or R¹¹ and R¹² taken together with a nitrogen to which they are both attached form a 3 to 7 membered heterocyclic ring wherein any one carbon atom of said heterocyclic ring can optionally be replaced with —O—, —S— or —NR^{a}—;
each Z is independently halogen, —O⁻, ═O, —OR^{b}, —SR^{b}, —S⁻, —NR^{b}₂, —N⁺R^{b}₃, ═NR^{b}, —CN, —OCN, —SCN, —N═C═O, —NCS, —NO, —NO₂, ═N₂, —N₃, —NHC(═O)R^{b}, —OC(═O)R^{b}, —NHC(═O)NR^{b}₂, —S(═O)₂—, —S(═O)₂OH, —S(═O)₂R^{b}, —OS(═O)₂OR^{b}, —S(═O)₂NR^{b}₂, —S(═O)R^{b}, —OP(═O)(OR²)₂, —P(═O)(OR^{b})₂, —P(═O)(O⁻)₂, —P(═OX)(OH)₂, —P(O)(OR^{b})(O⁻), —C(═O)R^{b}, —C(═O)X, —C(S)R^{b}, —C(O)OR^{b}, —C(O)O⁻, —C(S)OR^{b}, —C(O)SR^{b}, —C(S)SR^{b}, —C(O)NR^{b}₂, —C(S)NR^{b}₂, —C(═NR^{b})NR^{b}₂, where each R^{b} is independently H, alkyl, aryl, arylalkyl, or heterocycle;
each n is independently 0, 1, or 2;
each R^{a} is independently H, (C₁-C₈)alkyl, (C₂-C₈)alkenyl, (C₂-C₈)alkynyl, aryl(C₁-C₈)alkyl, (C₄-C₈)carbocyclylalkyl, —C(═O)R¹¹, —C(═O)OR¹¹, —C(═O)NR¹¹R¹², —C(═O)SR¹¹, —S(O)R¹¹, —S(O)₂R¹¹, —S(O)(OR¹¹), —S(O)₂(OR¹¹), or —SO₂NR¹¹R¹²;
wherein each (C₁-C₈)alkyl, (C₂-C₈)alkenyl, (C₂-C₈)alkynyl or aryl(C₁-C₈)alkyl of each R¹¹ or R¹² is, independently, optionally substituted with one or more halo, hydroxy, CN, N₃, N(R^{a})₂ or OR^{a}; and wherein one or more of the non-terminal carbon atoms of each said (C₁-C₈)alkyl is optionally replaced with —O—, —S— or —NR^{a}—.

4. The method of claim 3 wherein R¹ is H.

5. The method of claim 3 wherein R⁶ is H, CN, methyl, ethenyl, or ethynyl.

6. The method of claim 3 wherein R³ is OH, —OC(═O)R¹¹, or —OC(═O)OR¹¹.

7. The method of claim 3 wherein R⁸ is NR¹¹R¹² or OR¹¹.

8. The method of claim 7 wherein R⁸ is NH₂.

9. The method of claim 7 wherein R⁸ is OH.

10. The method of claim 3 wherein R⁹ is H.

11. The method of claim 3 wherein R⁹ is NH₂.

12. The method of claim 3 wherein each Y and Y¹ is O.

13. The method of claim 3 wherein R⁷ is H or

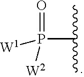

14. The method of claim 13 wherein R⁷ is selected from 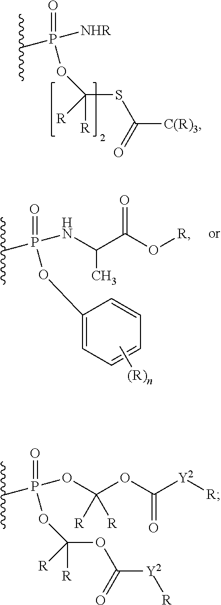
wherein Y² is, independently, a bond, O, or CR₂.

15. The method of claim 14 wherein R⁷ is

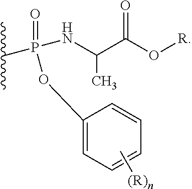

16. The method of claim 13 wherein R⁷ is H.

17. The method of claim 3 wherein W¹ and W² are each, independently, a group of the Formula Ia.

18. The method of claim 3 wherein the compound is 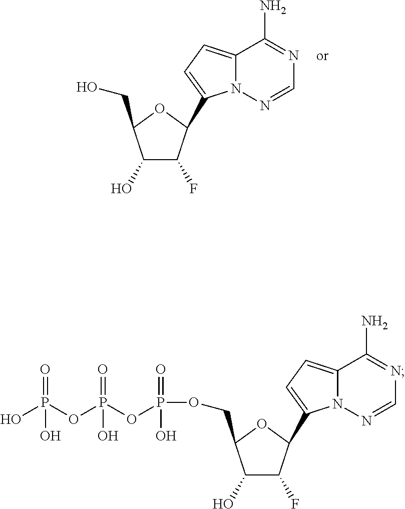 [SMILES: Nc1ncnn2c([C@@H]3O[C@H](CO)[C@@H](O)[C@H]3F)ccc12 ; Nc1ncnn2c([C@@H]3O[C@H](COP(=O)(O)OP(=O)(O)OP(=O)(O)O)[C@@H](O)[C@H]3F)ccc12]
or a pharmaceutically acceptable salt or ester thereof.

19. The method of claim 3 wherein the compound is 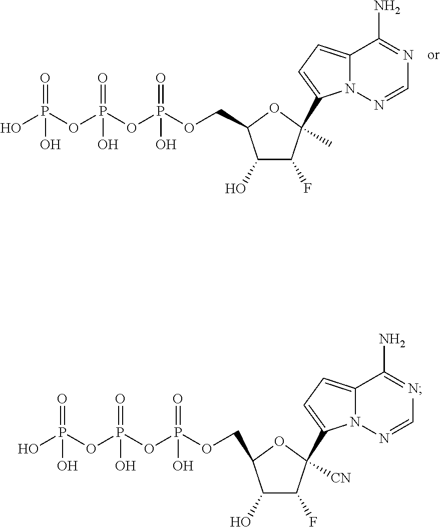 [SMILES: C[C@@]1(c2ccc3c(N)ncnn23)O[C@H](COP(=O)(O)OP(=O)(O)OP(=O)(O)O)[C@@H](O)[C@H]1F ; N#C[C@@]1(c2ccc3c(N)ncnn23)O[C@H](COP(=O)(O)OP(=O)(O)OP(=O)(O)O)[C@@H](O)[C@H]1F]
or a pharmaceutically acceptable salt or ester thereof.

20. The method of claim 3 further comprising a pharmaceutically acceptable carrier or excipient.

21. The method of claim 20 further comprising administering a therapeutically effective amount of at least one other therapeutic agent or composition thereof selected from the group consisting of a corticosteroid, an anti-inflammatory signal transduction modulator, a β2-adrenoreceptor agonist bronchodilator, an anticholinergic, a mucolytic agent, hypertonic saline and other drugs for treating Orthomyxoviridae virus infections; or mixtures thereof.

22. The method of claim 21 wherein the at least one other therapeutic agent is a viral haemagglutinin inhibitor, a viral neuramidase inhibitor, a M2 ion channel inhibitor, a Orthomyxoviridae RNA-dependent RNA polymerase inhibitor or a sialidase.

23. The method of claim 21 wherein the at least one other therapeutic agent is an interferon, ribavirin, oseltamivir, zanamivir, laninamivir, peramivir, amantadine, rimantadine, CS-8958, favipiravir, AVI-7100, alpha-1 protease inhibitor or DAS181.

24. The method of claim 23 wherein the compound of Formula I, Formula II and/or at least one therapeutic agent or mixtures thereof is administered by inhalation.

25. The method of claim 24 wherein the compound of Formula I, Formula II and/or at least one therapeutic agent or mixtures thereof is administered by nebulization.

26. The method of claim 3 wherein the Orthomyxoviridae infection is caused by a Influenza A virus.

27. The method of claim 3 wherein the Orthomyxoviridae infection is caused by a Influenza B virus.

28. The method of claim 3 wherein the Orthomyxoviridae infection is caused by a Influenza C virus.

29. The method of claim 3 wherein a Orthomyxoviridae RNA-dependent RNA polymerase is inhibited.

30. A compound having a structure 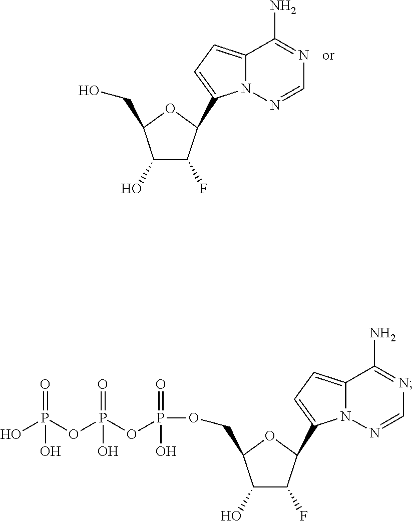 [SMILES: Nc1ncnn2c([C@@H]3O[C@H](CO)[C@@H](O)[C@H]3F)ccc12 ; Nc1ncnn2c([C@@H]3O[C@H](COP(=O)(O)OP(=O)(O)OP(=O)(O)O)[C@@H](O)[C@H]3F)ccc12]
or a pharmaceutically acceptable salt or ester thereof.
